SEQUENCE LISTING (34 sequences)

SEQ ID NO: 1 - DNA, 20306 bp, Pseudomonas fluorescens
ggtaccaacatcgacgacggcaccaaccgcggcgatggctccgagcgtgaaatctggaaccagttcaagtacgtggtccagagcggtccggccaaagacctgagcctgcgtgctcgtgcctcgtggctgcgcgtctccaataacgctgaccagtacaacgtaggcggtaacgaaatccgtctgttcgccgactacccgatcaacgttttctaattgatctggcgtcgcgcctgatgccaggcgctaaaaaaccccgactggttcggggtttttttatgcccgccagtgcgagacacgtttgttacaccacggcactgatgatgtacccggccccacctttatcccctcccggcgcatgcctacggcttcttctatatcccccaactcgcccttgccgagatggaaaagaacaacgccggccgcatcgtccacatcaccaccagcctggctgaccatgcaatcgatggcgtaccgtcggtacttgccaacctcaccaaaggcggcctgaactcagcgcccaaatcattggcgattgaatacgccaagcgcggtatccgagtgaatgcggtaagcccggggatcatcaagacgccgatgcatggcgaggaaagccatgctgcgctggggcaaatgcacccggttgggcacatgggggaggtgagtgaaatcgcccaggcgattatttatctggagaatgcggggttcgtgacgggggagattcttcatgtagacggcgggcagagtgccgggcattgatctacacgtcccggcttgattgggccgggcgttatcttgtcttggtgaggttgtgcgtaggctgggaggggggcacctgccatttggtactttaagaaattgagctcttccgggaaataggcagaaggtgacaaggtcggagaagctatcaaccaggtgagggcgtcggttgagcaaaaatgaagcgtggacactttttgggcattttggccaaaacgacgcccaagatttttcgtgttttccaaacccaagaaaccacaaagccccgcattgcggggcttcgagatatggtgccggcaccaggagtcgaacccgggacctactgattacaagtcagttgctctaccaactgagctataccggcgtgttagggcgacgattatagcgattggaaaggttctgtaaacccctgaattctgactatttttgcaaaacccaagcttttctcacactcgccccttttttcgaccagcaagggtagattcttctcttactgccgaagggtctaactcggcaatcttgtccaaactaaaggaagccccatatgaaacggactctctccctgtccctcgtcctcctcaccgccgctctcggcgcgtgctccacccatcagtcggccaacgaccccgcgctggttggcacttggaaaggcttgcgcaccgagaccggtaaatgccagttcctgtcgtggaccaataccctcaagcctgacggtcgtttcgttattaccttctaccgcgatgcgcagcagacgcaggtgatccacacggacacggttcgtgggcggcggccaatggttagaatgagctgcgcacagatcgcgtgcgttcgccggatgtgttcacctataagctgctggacgcagacactgttcactatgtgagtgtggcatcggaccccaccagcgattgccaggacgactaccagttcaccgagcgccgtgtccgctgacaaggcaggtttgcttcaaaaccgggcactcgggtgcccggccgtcacaagtttcaactcccttgcgcgcacgtgcccgcacctttctcgtagtccaatacctgtttttgccgggcgtgagcatcagcagcaaaagcctacagcctgactgcgatcaatttggtctagttaatggggcgtatcttccaggctcagcaggtttagcgctctggaggtgtctgccctgtgtcattcggcgtgggttttgcttcgtcgtcttggctgcctgcgcagcgatatacgggccgtggctttgcgggcggttgcggatcgaccgctcggcagtttcgtccttgaatacaggcgaggtttccgtattctgtcgcgcgatataaaccgtccgtttaatcgcggttgtgtgtaggcaaatttttgagacagtgtagggcaatcgaatatttgtttatgggtatgtcagattagtgccgtttctgaccactcagccaacaaataatacgataaatcagtgcaagttcatgttcttacagcgaatgcattctctctaaataatctgtcataaaaatggcgccacgccgttcgtcggataagattgagcgaaaagcatcaaaatactgacaccatcattgtgatatcacagaatgagcccgacaccacataactaaaaaaatacaaggtcagcctattgctgacctttttatcgcctttaaagcgcaaaccaggtggggaggggcagtgagtgagtcgagcagatgacatttcaaaactattcaacaagctgggtgccaacccgagtggctaccgcgagatcgacttcgtccacgagttcatcgaggacgatgtagaggtcctggaaaccccggcggtcgttcgagccctgcctgtgattgaagcgccgtcagcgcctttgctgcgtctgttggaagagctgagccaaggcgaagccgaccacctgcagccgcccgaagtggtggaagggcgggacggtgaggtgtactcggagcattccagccccaacgtcgtggtggttgtctcggtaaaaggcggcgttggccgcagcaccctgactgccgcgattgccagtggtttgcagcgtcaggggcgcccggcactggccctggacctggacccgcaaaacgccctgcgccaccacttgtgcctcggtctcgacatgcccggcgtgggcgcgaccagcttgctcaatgaaagctgggaagcgctgcccgagcgcggttttgccgggtgccgcctggtggcattcggtgctaccgaccacgagcagcaacagagcctgaatcgctggctgggccaggatgacgaatggctgagcaaacgcttggccggcctcaaattgaacggccaggacaccgtgatcatcgacgtcccggccggcaataccgtgtacttcagccaagccatgtcggtcgccgacgcagtgctggtagtggtgcagccggacgtggcgtccttcagcacgctcgatcagatggacagcgtgctcaagccctctctcaatcgcaaaaaaacgccacgacgcttctatgtgatcaaccaactggacggtgcccaccgcttcagcctggacatggccgaggtgttcaagacccgcctgggtgctgccctgttggggacggtccaccgcgaccccgcgttcagcgaagcccaggcctacgggcgtgatccccttgaccccaccgtcaacagtatcggcagccaggacatccatgccctgtgccgcgcattgctcgaacgaatcgactcggacctcccatgaccgacactacgtcctccacgcccttcgtcgaagggcgcgctgaacagcgcctaaatggcgccatcgcgcgcttcaatcggtggccttcggcgccgcgtacggtactggttgttgccagttgcgtgctcggcgccatgctgctgctgggcattatcagcgcgccgctcgacctctacagccaatgcctgtttgccgccgtgtgcttcctggcggtgctggtactgcgcaagatcccggggcgcctggcgatcctcgccttggtggtgttgtcgctggtggcgtcgttgcgctacatgttctggcgcctcacctccaccctcggctttgaaacctgggtcgacatgttcttcggctacggcctggtcgcggccgagttctacgccctgatcgtgctgatcttcggctacgtgcaaaccgcctggccgctgcgccgcacgccggtgtggctcaagactgagccggaagagtggccgacggtcgacgtgttcatcccgacgtataacgaggcgctgagcatcgtaaagctgaccattttcgccgcccaggcgatggactggcccaaggacaagctgcgcgtccacgtgctcgacgacggtcgccgcgacgacttccgcgaattctgccgcaaggtcggcgtgaactacatccggcgcgacaataacttccacgccaaggccggtaacctcaacgaagcgttgaaggtcaccgacggcgaatacatcgccctgttcgacgccgaccacgtgccgacgcgttccttcctgcaagtgagcctgggctggttcctgaaagatccgaagctggcgatgctgcaaacgccgcacttcttcttctcgccggacccgtttgaaaagaacctcgacacgttccgcgccgtgcccaacgaaggtgagctgttctacggcctggtgcaggacggcaacgacctgtggaacgccaccttcttctgtggctcctgtgccgtgattcgccgtgagccgctgctggaaatcggcggcgtagccgttgagaccgtgaccgaagacgcccacaccgcgctcaagctcaaccgcctgggctacaacaccgcttacctggccatcccacaagccgcgggcctggccactgaaagcctgtcgcgccacatcaaccagcgcattcgctgggcacggggcatggcgcagattttccgcaccgacaacccgctgctgggcaaaggcctgaagtggggccagcgcatctgttatgccaacgccatgcagcacttcttctatggtttgccgcgcctggtgtttttgaccgcgccgttggcctacctgattttcggcgccgaaatcttccacgcatcggcgctgatgatcgtcgcctatgtgttgccgcacttggtgcactccagcctgaccaactcgcggatccaggggcgcttccgtcactcgttctggaacgaggtgtacgagaccgtactggcctggtacatcctgccgccggtgctggtcgcgctggtcaatcccaaggcgggtggcttcaacgtcaccgacaagggcggcatcatcgacaagcagttcttcgactggaaactcgcccggccctacctggtgttgttggcggtcaacctgatcggcctcggcttcggtatccaccagttgatctggggcgatgcgtccactgccgtgacggtggcgatcaacctgacctggaccctctataacctgatcatcaccagtgccgccgtggccgtggcctcggaagcacgccaagtgcgttccgagccgcgtgtcagcgccaagctgccagtgagcatcatttgcgccgacggccgcgtgcttgatggcaccacccaggacttctcgcaaaacggcttcggcttgatgctttccgatggtcactcgatcacccagggtgaacgcgtgcaattggtactgtcgcgcaacggccaggacagcctgaaagacgcccgcgtggtgttcagcaagggcgcgcagatcggcgcccaattcgaagccctcagcctgcgccagcaaagcgaactggttcgcctgactttctcccgcgccgatacctgggccgcgagttggggcgccggtcagccggatacgccgctggcggccctgcgcgaagtcggctccatcggcatcggcggcttgttcaccctcggccgcgccaccctacacgaactgcgtctggccttgagccgcacccccacaaaaccgctagatacgctgatggacaagccatgacctcgaacatattcgctcgccctcatccgcgccgtgcactggcgctgatgatcgcctcgctgatgggcttcaacaccctcgcgcaagctgccgagcaagcggttgccaccgtgccggtacaaagtaccgacaccggctacagcctgaccctcaagcaattggggcgtcgcgacaccatgaacctgcaaggtgtggagtcgtccacagcgtaactttgatatccgtgccgatgaagtggtgaagggcgcgcaactgttgctcaagtacagctactcgccggcgctgctggccgatctgtcgcagatcaacgtgctggtcaacggcgaagtcgccgccagcctgccgctgcctaaagaaggcgcgggcacgccgcaagagcagttggtgcagatcccagcgcacttgatcaccgagttcaaccgcctgagcctgcagttcatcggccactacaccatgtcttgcgaagacccactgcacagcagcctgtgggccaagatcagcaacagcagtgagctgaaagtgcaggtcgagccgatcgtgctcaaagacgacttggccgtgctgccgctgccgttcttcgacaagcgtgacgcacgccaggtgagcctgccgttcgtgttcgccaccgctcccgacagcgccgcgctggaagccgccggcgctctgtcgtcgtggatcggcggcctcgccagctaccgcggcgcgacgttcccgaccaccctcggtgagttgccggccaaaggcaacgccatcgtgctggtgcaaactgccgacgcgatggacatacacggtgttgccgttgccaagccggccggcccgaccctcaccctcatcgccaacccgaacgacgccaacggcaagctgctgatcgtcaccggccgtgacggtgcagagctcaagcgcgccgcaacgcggtggtgctggcaacccggtattggccggtacagcgtggtgatcaccaagctggataccctcgcaccgcgtcgtccatacgatgccccgaactggctgccaagcaaccgcccggtccgcctgggcgagttgatcgaacagcaaaaactcagcgtgtcgggctacaacccaggcgcgatcagcgtcgatatgcgcctgccgccagacctgttcaactggcgtgaagagggtgtgccgctcaagctcaaataccgctacacgccgcagcaggtgtcgaccaactcgtcgctgctgatcggcctgaatgatcagttcatgaagtccgtggcattgccgtcggtgagcaacctgggcggcggccaaaccctgctcgaccagttgaagaaagacgaaagcctgccgcgtgaagtgacgaccctgttgccgatcagctcagcatcgcccaagtccaagctgcaagtgcgcttcatgtacgactacatcaaagaaggcgaatgccgcgacatcatcgtcgacaacatgcgcggttcggtcgacccggactcgaccctcgacgtcacgggctaccagcactacatcgccatgccgaacctgggcgtgttcaacgactcgggtttcccgttcacccgtttggctgacctgtcggagtcggccgtggtcatgcccgacaattacggtaccgacgagctgaccgcctacctgacggtgcttggccgttttggcgaagccaccggctacccggccactgcggtaaaagtagtgcaggccaaagacgtgcaaagcgttgccgacaaagacctgttggtgctcgctaccgctgccaaccaaccgctgctcaagcagtggcagcaatacctgccggcgaccagcgatggcgagcaacaccagttcctgctgtctgacctgccacgttatgtgcgcagctggatcagcccggacccggcggccaaccaacacccggccaacaccggcattaccttcaagggcctgagcaacagcacgtggctggcgggtttccaatcgccgctcaagagcggtcgcagcgtggtgctgatcgccagcaaccagcctcagggcctgctggaagcgaccaacgcgctgatcggcggtgacgactataaagactcgatccaaggcagcctggcggtggtgcaaggcacgcaaatcagctcgctggtaggcgatgagcagtactacgtcggcaagctcaactacttcaaattcatgcagtggcaactgtcgcagaacctgggttggatgctgttgatcaccttcctcggcctggccgtggtcaccagcctgatctacttgtcgctgcgtgcccgtgcaaaacggcggttggcatgagcccgcttaagtgcatggccctggcggcgctgggcgctgtgatgttcgtcggtagcgcgcaagcgcaaacctgcgactggccgttgtggcagaactacgccaagcgcttcgtgcaggatgatgggcgcgtgctgaactcgtccatgaaacccaccgagagcagctcggaaggccaatcctatgcgatgttctttgccctggtcggcaacgaccgtgctagcttcgacaagctgtggacctggaccaaggccaacatgtcgggcgccgacatcggccagaacctgcccggctggttgtggggcaaaaaagccgataacacgtggggtgtgatcgacccgaactccgccagcgacgccgacttgtggatggcctacgccttgctcgaagcggcacgcgtgtggaatgcaccgcagtaccgtgccgacgcgcaattgttgctggcaaacgttgaacgcaacctgatcgtgcgtgtgcctggcctcggcaagatgctgttgccaggcccggtgggctacgtgcacgccggtggcctgtggcgcttcaacccgagctatcaggtgctggcgcaactgcgtcgcttccacaaagaacgcccgaatgccggctggaatgaggtggccgacagcaacgccaagatgcttgccgatacggccagcaaccctcacggcctcgcggccaactgggtcggttaccgcgccaccagcgccaacaccgggctgttcgtggtcgacccgttttccgatgacttgggcagctatgacgcgatccgcacttacatgtgggccggcatgaccgccaagggcgatccgctggcggcgcccatgttgaaatccctgggcggcatgacgcgggccaccgcagcatcggccaccggctacccaccggaaaaaatccatgtactgaccggcgaagtcgagaaaaacaacggctatacgccgatgggcttctcggcctccaccgttgccttcttccaggctcgcggcgaaaccgcactggcgcagttgcagaaggccaaggtcgacgacgcgctcgccaaagccctggccccttcggcgccggacaccgcgcagccgatctactacgactacatgctcagcctgttcagccagggctttgccgatcagaagtaccgttttgaacaagacggtacggtcaaactttcctgggaggccgcatgcgccgtcacacgctagccattgcgattctcgccgccctggcctccaccgccagcgtcgctgaaaccagcgacccgcagtccctgctgattgagcagggctactactggcagtcgaaaaagaaccccgaacgcgctctggaaacctggcagaaattgctgcgcctgagcccggaccaaccggatgcgctgtacgggatcggcctgatccaggtgcaacagaaccacccggccgaagcgcaaaagtacctggcccgtttgcaagcgttgagcccggtgccgcgccaggccttgcaactggaacaggacatcaccgttgcggttccggacaacgccaagttgctggaacaggcgcgtgaactgggcgagccggaggccgagcgtgaacaggctgtggcgttgtatcgccagattttccagggccgtcagcctcaaggcctgatcgcccgcgagtactacaacaccctgggctttaccgccaaaggcagcagcgaggccatcgccggcctgcagcgcctgacccgtgaacgcccgaacgacccgatcgttgcgctgttcctggccaagcacctggcgcgtaacccggccacccggcctgacggtatacgggccctggctaaactggcatcgaacaacgacgtgggcggcaacgctgacgaaacctggcgcttcgcgctggtctggctcggcccgccgaaaccggaccaggtttcgctgttccagcagttcctcaccgtgcacccggatgacagcgagatccgcgcgctgatgaacaaaggcatcgcccaaggtaaaggcggtggcacctggcagcgtgatccgcagatgaccaaggctttcaaggcgttggatgacggcgacctgaaaaccgccgagcctctgctggcggcgcgccttgcgcaaaagtccaatgacgttgatgccctcggcggcatgggcgtgttgcgtcaacagcaagagcgctacagcgaggcggaaaactacctggtccaagccacgcgcttgccgggtggcgctgcttggcagtcggccttgaatgatgtgcgctactggaatctgatcagccaaagtcgtgacgcccagcgtgccggtcgtagcgcccaggcccgcgacctggtggcccaggccgaacgcctgaaccctggccagcctggcgcggctattgcgctggcgggcttccaggcccaggacaaccagttcgacgacgccgaagcgggctaccgcaaagtgctggctcgccaccctggcgacccggatgccttgagcggcttgatcaacgtgctgtcccagtcgggccagccggatgaagccttgaagctgatcgactcggtatcgccggcccagcgcgccaagttcgcaccgagtgtgaaaatcaacgcgttgcgcgcgacccaggtgggcaagctggccgagcagcggggggatctgaaggccgcgcaagccgcctatcgccaggccctcgatgccgacccggaaaacccctggacccgcttcgccttggcgcgcatgtacctgcgcgacgggcagatccgcaacgcgcgagccttgatcgacggcttgctcaagtcccagcccaaccaacccgatgcgctgtacaccagcaccttgttgtcggcgcaattgagcgagtggaagcaagccgaggccaccttggggcgtatccccacggcacagcgcacggcggacatgaacgaactggcaactgacatcgcgctgcaccaacagaccgacatcgctatcgaaaccgctcgccgtggccagcgcccggaagccttggcgttgctcgggcgttccgaaccgctgacccgcaataaacctgagcgtgtggccgtgttggccgccgcctatgtggaagtgggggctgcgcagtacggtctggacatgatgcagaaggtggtggagaacaaccccaacccgacggtcgaccagaagctgctgtacgccaacgtgctgctcaaggccaacaaatacagtgaggcgggcgagatcctgcgcgaagtgcaaggccagcctttgaccgagaccggccgccagcgctacgacgatttgatctacctgtaccgggtcaagcaggccgacgccctgcgtgagaagaacgacctggtggcggcttacgacatgctgtccccggccctggcccagcgcccgaacgacgcgttgggcgtcggcgccctggcgcggatgtacgccgccagcggtaatggcaagaaggccatggagctgtacgcgccgttgatccagcagaaccccaataatgcgcgactgcaactgggccttgcggacatcgccctgaaaggtaatgaccgtggcttggcgcaaagcgccagtgacaaggcgttggccctggagccgggtaacccggagatcctcacctcggccgcgcgcatctatcagggcctgggcaagaacagcgaagccgcggaactgctgcgcaaggccctggcgattgaaaacgccatgaaggccaagacccaggtagcccaggccagcgctccaggtacgtcgtataacccgttcgtgggcttgccgggccagcgtcgccaggtcaccgacctgaccgttgccggcgcggtaccgccaccgatcgatgcaccgaccaagtccgtgacgtccaacgcgtttgcgagtgccacgtccaacgacttgagcgacccgtttgtgccgccatcgagcattgcgtcgatcgacagccccgagctgagcccggcgcgtcgtgcgctggacactatcctgcgtgaccgtaccggttatgtggtgcagggcttgagcgtacgcagcaacaacggtgagaaaggcttgagcaaaatcaccgatgtggaagcgccgtttgaagcgcggatgccggtgggggataacactgtggcgctgcgggtgacgccggtgcatttgagtgctggtagcgtcaaggctgagtcgttgtcgcgctttggtaaaggcggcacagagccggcgggctctcagagcgatagcggtgtcggtttggccgtggcgtttgaaaacccggatcaaggcctcaaggctgacgtcggcgtgagccctctgggcttcctctacaacaccctcgtgggcggcgtgagcgtgtcgcgtccgttcgaggccaattcgaacttccgctacggcgccaacatctcgcggcgcccggtcaccgacagcgtcacttcctttgccggctccgaagacggcgcgggcaacaagtgggggggcgtcaccgccaacggcggccgtggcgagctgagctatgacaaccagaaactgggcgtctacggctacgcgtcgttgcatgagttgctgggcaacaatgttgaagacaacacccgcctggagttgggcagcggtatctactggtacctgcgtaacaacccgcgcgacacgttgacgctgggtatcagtggttcggcgatgaccttcaaggaaaaccaggacttctacacctacggcaacggcggttacttcagcccgcagcgctttttctccctcggcgtaccgattcgttgggcgcaaagctttgatcgcttcagttaccaggtaaaaagttcggtgggcctgcagcatatcgcgcaagatggcgcggattatttccctggcgacagcacgcttcaagccaccaaaaataatccaaagtacgacagcaccagcaagactggcgtcggctacagcttcaacgccgccgccgaataccgcttgagctcgcgcttctacctgggcggcgaaatcggtctcgacaacgcccaggactaccgccagtacgccggtaatgcttatctgcgctacttgttcgaagacctgagcgggcctatgcccttgccggtcagtccgtaccgttccccttattccaactgattaatcggagtcattcatgcctgtttctgcgattgccggcctaaccatgctggtattgggcgaaagtcatatgagctttcccgattcgttgctcaacccgctgcaagacaacctcaccaagcaaggcgcggtggttcactccatcggtgcgtgcggtgccggtgcggcggattgggttgtgccgaaaaaagtcgaatgcggcggcgaacgcacgcccaccggcaaggccgtgatctatggcaaaaacgccatgagcaccacgccgatccaggagctgatcgccaaagacaaacccgacgtggtcgtgttgatcatcggcgacaccatgggctcctacaccaacccggtgttccctaaagcctgggcctggaaaagcgtgacctcgctgaccaaagccatcaccgacaccggcaccaagtgcgtgtgggtcggcccgccatggggcaaggtcggttcgcagtacaagaaagacgacacccgcaccaagctgatgtcgtcgttcctcgccagtaacgtggcgccgtgcacctacatcgactcgctgacgttctcgaagccaggcgagtggatcaccaccgacggccagcacttcaccatcgacggctaccagaagtgggccaaggccatcggtacagccttgggtgacctgccgccatcggcctacggtaaaggaaacaaataatgaaacggatgaccctgggcctggcgaccttgctcgccagtgtcagcgcctactgtgccgacatcccgctgtacccgaccggcccggagcaagacgcagcattcctgcgttttgccaacggcacccctggcgaattgaagctggtggcggacggttccaaggccagcctggtgttgagcggcgacaaagccgtgtcggcgttcctgccggttgtcggcggcgacaaaccgatcaaaggcgtattgagcagcggcggcaaaaacgctgatttctcggtgaaagtggcgccgggcgaattcgccacggtggttgcactggtcgacgccaagggcgccacgcgccaattggtggtgcgtgaagtgccggacgacttcaatgcgctcaaagcctcactggcgttcatcaacgccgacgccacctgcgccgacgccagcctggaagccgtggcgcaaaaggccgagctgttcaagcaggtcgctgaaggtgccgtgcagcggcgcatgatcaacccggtggaattgtcggtgcagctcaaatgcgccggctcgccggtgggccagccgttgacgttcactctcaaggcgggcgagcgctacagcgtattggccgttccttcagacacaggctcgaagttgctgttcgcctctgacgcactcgctaactgatcgggcccgacaccaccatggtctttgcctcactcgaattcctcacgctgttcctgccggccttcctgttgatctatgccctggctcgtccgagctggcgcaacgtgatcctgttgatcggcagctggttgttctacggctggttgagcccgttgttcctgttcctgcacatggtgttgaccgtggtggcatgggtcggcggcttgctggtggaccgctcccgtgaggacggcaaaggccgggtgcgcctgttgatcgcgttgatcgtgttcaacacggccgtgctgtgttggtacaagtacgccaacatcgtcgccggcactgtgagcgaggtgatcacctggtacggtgcgatgccgttggactggcagcgcgtggccttgccggcgggcttgtcgttcatcgtcttgcaggcgatttcctacctggtggatgtgcaccgtcatacggtgccggtggagcgcagcttcattaactacgccacctatatctcgatgttcgggcactcgattgcgggcccgatcatccgttacgactgggtccgccgtgagctgaaccagcggtatttcaactgggcgaatttctccctgggcgcacggcgcttcatgatcggcatgggcatgaaagtgctggtggccgacaccttgtcgccgctggtggacattgccttccacctggaaaaccccagcctggtggacgcctggatcggttgcttggcgtattcgctgcaactgtttttcgacttcgccggctacagcgccatggccatcggcttgggcttgatgctgggcttccacttcccggaaaacttcaaccggccgtacctgcagcagcatcagacttctgcggcgttgcacttgtcgtgtcagctgctgcgcgactacctgtacatcgcgctggcgggtaaccgtgacggtgcctggcgcacctaccgcaacctgttcctgaccatggcgattgccgggttgtggcacggcggcgacagctggaactacctgttgtggggttcggcccacggcgtggcgctgtgtgttgaccgtgcatggtcgcggtcgagcttgccgagcattccgccggtgctgtcgcacgttctcacgctgctgtttgtgtgcctggcctggacgttgttccgcgcgccggacttccattcggcactgaccatgtacgccggccagttcggtctgcacggcatggccttgggtgacgcgatggccgtggccatgcgcccggcccatggcatggcggcgttgctggggctggtgtgcatcatcgcgccgatctggcaggtgcgttgcgaacagcgcttcggcacccagccgtggtttgtggtggcggcctcgctgtggccggtggccgggtttgtgttgtcgttcgcgctgattgccagccgcgacgccgtaccgtttctgtactttcagttctaaggacgcccgaccatgcctgctcctaccgcgccgactccaccgagcgacttggccgttcgcaccagccccttggcggcctgggtgctggtgccctttttggcggcggggctgctgtcctgcgtttggctgatggtgaaggggccgatcagttatgtgccggccaaggtcgacagcgacatgttgctgcacggtgacctgacccaccggtttgccaaggaattggccaaggcgccgatggcgatccaggccgccaacctggagcgcggcggcagctggctggcgtttggcgacaccgggccgcgcgtacgtccgggttgccccggctggctgtttatcagcgatgagctgcgcatcaaccggcatgccgaggccaatgcccagaccaaggcccaggcggtgatcgacctgcaaaaacaactgggtcaaaaaggtatcgacctgcaagtggtggtggtgccggacaagagccgcatcgccgccgcccaacgctgtggcctgtaccgcccggcggtgctcgataaccgggtgcgggattggaccgccatgttgcaggccgccggcgtttccgcgttggacctcaccgaaacattgaaaccgctgggcgccgaagcctacctgcgcaccgacacccactggagcgaaatcggctccaacgcgggggccaaggccgtggcgcagcgtacccagcagcgcggcatcaaggccacgccggagcaaaccttcgacatcacccaggcgccgctcgccgtgcgtccgggcgacttggtgcgcctggccggtctcgactggttgccgcccacgttgcagccgccaggggaatcggtcgcggccagcacgacccatgaaaccggtggtgcgaccagtaatgccgacgatctgtttggcgatgccggcctgcccaatgtggcgctgatcggcacgtcgttctcgcgtaactccaactttgtcggcttcctgcaaaaggccctgaatgcgcctgtgggcaatttcagcaaggacggcggcgaattctccggtgcggccaaggcgtacttcgacagcccggcgttcaagcaaaccccgcctaagttgttgatttgggagattccagagcgcgatttgcaaacgccatacgatgtcatcacgattggccagtaatttgacgattgtccgtttgtctaaaaaataaacagcgcaagcatcaaaataatgacactcttgtgagcttgaagcagactgcctgcacaagggcgacgacaaaaagacctgcggtaagcagccgggtgagctggccttttcacgtcgttaagcttcgccgtgatgaggaatccgctatgagctttacagatggtcttctcgttctgctggggaaagacgtcagccgtgacgcccaagggctggatgcgcggctgcatttttttggcagcattcccgtggatgacggcaccccgtttccccctcaagcccccagtgcgcaggcgcaatcccagtccgtggataagggggtacgacgtccgtgcgtggtggcgttggtgtcggtcaatggcggcgtggggcgcagtaccttggcgacggccttgagcagcggtttgcagcgcctgggtgagtcggtggtcgccgtggacctggacccacagaacgccctgcggatgcacttcggtgtcagccccgcctcgccgggtatcggcccgacaagcctgcgcaatgcgcagtgggacaatatccagcagccaggctttgtcggcagtcgtgtgatcacctttggcgacaccgatatgcgccagcaggacgacctgcaacgttggctcaaacatgaaccggactggttggcgcagcgcctgtctgcattgggcctgagcgcccgtcataccgtgatcatcgatacccccgccggcaataacgtctacttccatcaagccttgagcgtggccgacgtggtgctggtgattgcccaagccgacgccgcctccctgggtacgctggaccaactcgacgggctgttggcaccgcaccttcagcgcgaacgcccaccgcatgtgcactttgtgatcaaccaactggatgaggacaatgccttcagcctggatatggtggaggcgttcaagcagcggctcggcaccagagagcctctagaggtgcaccgcgatatggcgatcaagcgaggcgctggcgtttggtatcgacccattggatagccaggcattgagcttggcgggggacgatatcaccgagctttgccggctgctgattgcacgtaaaaaacggctttaaattgcgctttattcgcttaacgccagatgcttatgcacaatcggttggagggcaatgtcccgaaacagccgttgtgtggaggcattctcatcacgccgcaactgcctgttttgagtgcgttttattttgtgaacaaaaaatgaccagcctggcttttggcctgtaacgtagatggctacgggctctgtaaagaattcatcaacagagttatccacaggatgtgccgcgacaatttcgccctttaatcgcgctcggcaagcaccatcaagttgcgcggcgtcaaggccggttcgcagaaggtgccgacttccaccctgtagccgttttcgccgagaaacagtgcacgatccagcaccagccataactccagcggccgccgaaacagaccacgcaccaattccaggtttctgacctctgccaggcgtcgccagccatacgcctctaaaccggcccaatcctgttcgtctgtggataaccctttgagctttgccagcgcgtggcagtagtccgcgaagggcctgtccagccagcttcgcgggcagcgacggcgtggacaggtactcgtcacagccacgcaactggcgttgcagttgatcgaaacccaggcgtcgcgccatggacgtgtcccgctgcaaacgcacccgtttgcccgcggtgacggtttcgctcagcggcaggccgaggtcatcgatcgacagttgcaggggtgaagcacgaccggcgctggacagtggctggtaggcctgggcgtttatgcggttgtagcagcagggcgccagcgccaactgtttgcagccggcggcgctggccaggtgcagcaggcgtacatgcaggtcgccacaggcatgcagggcgacgggcgtgtgttcggggccgatagccacagcggccatcacgtcttgcaggcaatgcgtgacggcaggccgtgatgctcgctcaaggcctggcctgcggcgatcagcgccgggtcgtattccaggcaggtcaatgcttgatctgcctgcaacaggcgacgacccagatggccttttcccgcacaccagtccagccagtgcctgggcttttgcgcaaactgcagcgcggcgccgaacgcttcgatttgtgcccatttacggccaggtacatcgacgctaaggcgatggcgagcgggcgccaagggctgcatcggtaacttatccacagcactgagctgccgggcttgcgcggcaagctgcggaaacggcgcgggtgccgccaggtcgtggggctgattgtggctggcttcggcagcggctaacgaacgtcggcgtagccactgggcgagttcggggtgctgggtttcccaaggtaaatacaggtgcgtaaagggccgtggtttccacagcccttggtggtcggtcaggaatgcatccagcgcctggaagcgtgcctcaacgtccttgacaggcatcaacgcgcagccagcgttccagcagtttgaagccgcgtaccagcaggtacgacatcagcaggtagaacacaccagcggtgaagaacatatccactgtcaggtagttgcgcgcaatgatcgtcctggccatgccggtcagctcaagcagcgttaccgtactcgccagcgagctggccttgagcatcaggatcacttcgttgctgtaggccggcaagccgatacgcgcggcgcgcggcaggatgatgtagaacaacgtcttgggcttggacatgcccagggcgcgagcggcttcgatctcgcccgggggaatagcctggatcgcaccgcgcaggatctcagcgatgtacgcggcggtgtggagggtcatggtggccgtggcacaccagaacggatcacgcaggtacggccacatggcgctggtgcgcacggcatcgaactgggccaggccgtagtagaccaggaacagttgcaccagcagcggtgtgccacggaagaaaaagatgtaggcgtagggcagcgaacgcacataccaacgcttggacgcacgagcgacgcccagcggaatcgcgagtatcaggccaagaatgacagcgatgcccaccagttccagggtcaggatggcgccttggataaactttggcagccacttgatgatgacttcccattccattacgtcgtcctcacgaagccgcgggcggcgcgttgttccaagaagtgcatgccgaccatcgacagcgaggtcagggcccaggtagatgaaggccgcgaccagatagaaggtgaagggttgccggtaacggtgaccgcatctgcgcgtgacgcatgattcttcaggccgatgaccgtaccagcgcaggtcttataggatcatgacagtaccagctgcagc

SEQ ID NO: 2 - protein, 344 aa, Pseudomonas fluorescens
VSRADDISKLFNKLGANPSGYREIDFVHEFIEDDVEVLETPAVVRALPVIEAPSAPLLRLLEELSQGEADHLQPPEVVEGRDGEVYSEHSSPNVVVVVSVKGGVGRSTLTAAIASGLQRQGRPALALDLDPQNALRHHLCLGLDMPGVGATSLLNESWEALPERGFAGCRLVAFGATDHEQQQSLNRWLGQDDEWLSKRLAGLKLNGQDTVIIDVPAGNTVYFSQAMSVADAVLVVVQPDVASFSTLDQMDSVLKPSLNRKKTPRRFYVINQLDGAHRFSLDMAEVFKTRLGAALLGTVHRDPAFSEAQAYGRDPLDPTVNSIGSQDIHALCRALLERIDSDLP

SEQ ID NO: 3 - protein, 739 aa, Pseudomonas fluorescens
MTDTTSSTPFVEGRAEQRLNGAIARFNRWPSAPRTVLVVASCVLGAMLLLGIISAPLDLYSQCLFAAVCFLAVLVLRKIPGRLAILALVVLSLVASLRYMFWRLTSTLGFETWVDMFFGYGLVAAEFYALIVLIFGYVQTAWPLRRTPVWLKTEPEEWPTVDVFIPTYNEALSIVKLTIFAAQAMDWPKDKLRVHVLDDGRRDDFREFCRKVGVNYIRRDNNFHAKAGNLNEALKVTDGEYIALFDADHVPTRSFLQVSLGWFLKDPKLAMLQTPHFFFSPDPFEKNLDTFRAVPNEGELFYGLVQDGNDLWNATFFCGSCAVIRREPLLEIGGVAVETVTEDAHTALKLNRLGYNTAYLAIPQAAGLATESLSRHINQRIRWARGMAQIFRTDNPLLGKGLKWGQRICYANAMQHFFYGLPRLVFLTAPLAYLIFGAEIFHASALMIVAYVLPHLVHSSLTNSRIQGRFRHSFWNEVYETVLAWYILPPVLVALVNPKAGGFNVTDKGGIIDKQFFDWKLARPYLVLLAVNLIGLGFGIHQLIWGDASTAVTVAINLTWTLYNLIITSAAVAVASEARQVRSEPRVSAKLPVSIICADGRVLDGTTQDFSQNGFGLMLSDGHSITQGERVQLVLSRNGQDSLKDARVVFSKGAQIGAQFEALSLRQQSELVRLTFSRADTWAASWGAGQPDTPLAALREVGSIGIGGLFTLGRATLHELRLALSRTPTKPLDTLMDKP

SEQ ID NO: 4 - protein, 689 aa, Pseudomonas fluorescens
VWSRPQRNFDIRADEVVKGAQLLLKYSYSPALLADLSQINVLVNGEVAASLPLPKEGAGTPQEQLVQIPAHLITEFNRLSLQFIGHYTMSCEDPLHSSLWAKISNSSELKVQVEPIVLKDDLAVLPLPFFDKRDARQVSLPFVFATAPDSAALEAAGALSSWIGGLASYRGATFPTTLGELPAKGNAIVLVQTADAMDIHGVAVAKPAGPTLTLIANPNDANGKLLIVTGRDGAELKRAATRWCWQPGIGRYSVVITKLDTLAPRRPYDAPNWLPSNRPVRLGELIEQQKLSVSGYNPGAISVDMRLPPDLFNWREEGVPLKLKYRYTPQQVSTNSSLLIGLNDQFMKSVALPSVSNLGGGQTLLDQLKKDESLPREVTTLLPISSASPKSKLQVRFMYDYIKEGECRDIIVDNMRGSVDPDSTLDVTGYQHYIAMPNLGVFNDSGFPFTRLADLSESAVVMPDNYGTDELTAYLTVLGRFGEATGYPATAVKVVQAKDVQSVADKDLLVLATAANQPLLKQWQQYLPATSDGEQHQFLLSDLPRYVRSWISPDPAANQHPANTGITFKGLSNSTWLAGFQSPLKSGRSVVLIASNQPQGLLEATNALIGGDDYKDSIQGSLAVVQGTQISSLVGDEQYYVGKLNYFKFMQWQLSQNLGWMLLITFLGLAVVTSLIYLSLRARAKRRLA

SEQ ID NO: 5 - protein, 436 aa, Pseudomonas fluorescens
VATVAEPGLDAVDHLPRPGRGHQPDLLVAACPCKTAVGMSPLKCMALAALGAVMFVGSAQAQTCDWPLWQNYAKRFVQDDGRVLNSSMKPTESSSEGQSYAMFFALVGNDRASFDKLWTWTKANMSGADIGQNLPGWLWGKKADNTWGVIDPNSASDADLWMAYALLEAARVWNAPQYRADAQLLLANVERNLIVRVPGLGKMLLPGPVGYVHAGGLWRFNPSYQVLAQLRRFHKERPNAGWNEVADSNAKMLADTASNPHGLAANWVGYRATSANTGLFVVDPFSDDLGSYDAIRTYMWAGMTAKGDPLAAPMLKSLGGMTRATAASATGYPPEKIHVLTGEVEKNNGYTPMGFSASTVAFFQARGETALAQLQKAKVDDALAKALAPSAPDTAQPIYYDYMLSLFSQGFADQKYRFEQDGTVKLSWEAACAVTR

SEQ ID NO: 6 - protein, 1279 aa, Pseudomonas fluorescens
MRRHTLAIAILAALASTASVAETSDPQSLLIEQGYYWQSKKNPERALETWQKLLRLSPDQPDALYGIGLIQVQQNHPAEAQKYLARLQALSPVPRQALQLEQDITVAVPDNAKLLEQARELGEPEAEREQAVALYRQIFQGRQPQGLIAREYYNTLGFTAKGSSEAIAGLQRLTRERPNDPIVALFLAKHLARNPATRPDGIRALAKLASNNDVGGNADETWRFALVWLGPPKPDQVSLFQQFLTVHPDDSEIRALMNKGIAQGKGGGTWQRDPQMTKAFKALDDGDLKTAEPLLAARLAQKSNDVDALGGMGVLRQQQERYSEAENYLVQATRLPGGAAWQSALNDVRYWNLISQSRDAQRAGRSAQARDLVAQAERLNPGQPGAAIALAGFQAQDNQFDDAEAGYRKVLARHPGDPDALSGLINVLSQSGQPDEALKLIDSVSPAQRAKFAPSVKINALRATQVGKLAEQRGDLKAAQAAYRQALDADPENPWTRFALARMYLRDGQIRNARALIDGLLKSQPNQPDALYTSTLLSAQLSEWKQAEATLGRIPTAQRTADMNELATDIALHQQTDIAIETARRGQRPEALALLGRSEPLTRNKPERVAVLAAAYVEVGAAQYGLDMMQKVVENNPNPTVDQKLLYANVLLKANKYSEAGEILREVQGQPLTETGRQRYDDLIYLYRVKQADALREKNDLVAAYDMLSPALAQRPNDALGVGALARMYAASGNGKKAMELYAPLIQQNPNNARLQLGLADIALKGNDRGLAQSASDKALALEPGNPEILTSAARIYQGLGKNSEAAELLRKALAIENAMKAKTQVAQASAPGTSYNPFVGLPGQRRQVTDLTVAGAVPPPIDAPTKSVTSNAFASATSNDLSDPFVPPSSIASIDSPELSPARRALDTILRDRTGYVVQGLSVRSNNGEKGLSKITDVEAPFEARMPVGDNTVALRVTPVHLSAGSVKAESLSRFGKGGTEPAGSQSDSGVGLAVAFENPDQGLKADVGVSPLGFLYNTLVGGVSVSRPFEANSNFRYGANISRRPVTDSVTSFAGSEDGAGNKWGGVTANGGRGELSYDNQKLGVYGYASLHELLGNNVEDNTRLELGSGIYWYLRNNPRDTLTLGISGSAMTFKENQDFYTYGNGGYFSPQRFFSLGVPIRWAQSFDRFSYQVKSSVGLQHIAQDGADYFPGDSTLQATKNNPKYDSTSKTGVGYSFNAAAEYRLSSRFYLGGEIGLDNAQDYRQYAGNAYLRYLFEDLSGPMPLPVSPYRSPYSN

SEQ ID NO: 7 - protein, 221 aa, Pseudomonas fluorescens
MPVSAIAGLTMLVLGESHMSFPDSLLNPLQDNLTKQGAVVHSIGACGAGAADWVVPKKVECGGERTPTGKAVIYGKNAMSTTPIQELIAKDKPDVVVLIIGDTMGSYTNPVFPKAWAWKSVTSLTKAITDTGTKCVWVGPPWGKVGSQYKKDDTRTKLMSSFLASNVAPCTYIDSLTFSKPGEWITTDGQHFTIDGYQKWAKAIGTALGDLPPSAYGKGNK

SEQ ID NO: 8 - protein, 221 aa, Pseudomonas fluorescens
MKRMTLGLATLLASVSAYCADIPLYPTGPEQDAAFLRFANGTPGELKLVADGSKASLVLSGDKAVSAFLPVVGGDKPIKGVLSSGGKNADFSVKVAPGEFATVVALVDAKGATRQLVVREVPDDFNALKASLAFINADATCADASLEAVAQKAELFKQVAEGAVQRRMINPVELSVQLKCAGSPVGQPLTFTLKAGERYSVLAVPSDTGSKLLFASDALAN

SEQ ID NO: 9 - protein, 468 aa, Pseudomonas fluorescens
MVFASLEFLTLFLPAFLLIYALARPSWRNVILLIGSWLFYGWLSPLFLFLHMVLTVVAWVGGLLVDRSREDGKGRVRLLIALIVFNTAVLCWYKYANIVAGTVSEVITWYGAMPLDWQRVALPAGLSFIVLQAISYLVDVHRHTVPVERSFINYATYISMFGHSIAGPIIRYDWVRRELNQRYFNWANFSLGARRFMIGMGMKVLVADTLSPLVDIAFHLENPSLVDAWIGCLAYSLQLFFDFAGYSAMAIGLGLMLGFHFPENFNRPYLQQHQTSAALHLSCQLLRDYLYIALAGNRDGAWRTYRNLFLTMAIAGLWHGGDSWNYLLWGSAHGVALCVDRAWSRSSLPSIPPVLSHVLTLLFVCLAWTLFRAPDFHSALTMYAGQFGLHGMALGDAMAVAMRPAHGMAALLGLVCIIAPIWQVRCEQRFGTQPWFVVAASLWPVAGFVLSFALIASRDAVPFLYFQF

SEQ ID NO: 10 - protein, 374 aa, Pseudomonas fluorescens
MPAPTAPTPPSDLAVRTSPLAAWVLVPFLAAGLLSCVWLMVKGPISYVPAKVDSDMLLHGDLTHRFAKELAKAPMAIQAANLERGGSWLAFGDTGPRVRPGCPGWLFISDELRINRHAEANAQTKAQAVIDLQKQLGQKGIDLQVVVVPDKSRIAAAQRCGLYRPAVLDNRVRDWTAMLQAAGVSALDLTETLKPLGAEAYLRTDTHWSEIGSNAGAKAVAQRTQQRGIKATPEQTFDITQAPLAVRPGDLVRLAGLDWLPPTLQPPGESVAASTTHETGGATSNADDLFGDAGLPNVALIGTSFSRNSNFVGFLQKALNAPVGNFSKDGGEFSGAAKAYFDSPAFKQTPPKLLIWEIPERDLQTPYDVITIGQ

SEQ ID NO: 11 - protein, 324 aa, Pseudomonas fluorescens
VSLKQTACTRATTKRPAVSSRVSWPFHVVKLRRDEESAMSFTDGLLVLLGKDVSRDAQGLDARLHFFGSIPVDDGTPFPPQAPSAQAQSQSVDKGVRRPCVVALVSVNGGVGRSTLATALSSGLQRLGESVVAVDLDPQNALRMHFGVSPASPGIGPTSLRNAQWDNIQQPGFVGSRVITFGDTDMRQQDDLQRWLKHEPDWLAQRLSALGLSARHTVIIDTPAGNNVYFHQALSVADVVLVIAQADAASLGTLDQLDGLLAPHLQRERPPHVHFVINQLDEDNAFSLDMVEAFKQRLGTREPLEVHRDMAIKRGAGVWYRPIG

SEQ ID NO: 12 - protein, 333 aa, Pseudomonas fluorescens
MNDLQIDDIKTDENAAMVLLVDDQAMIGEAVRRGLAHEENIDFHFCADPHQAIAQAIRIKPTVILQDLVMPGLDGLTLVREYRNHPATQNIPIIVLSTKEDPLIKSAAFSAGANDYLVKLPDNIELVARIRYHSRSYMTLLQRDAAYRALRVSQQQLLDTNLVLQRLMNSDGLTGLSNRRHFDEYLELEWRRAMRDQTQLSLLMIDVDFFKTYNDSFGHVEGDEALRKVAATIREASSRPSDLPARYGGEEFALVLPNTSPGGARLVAEKLRMAVAALKIPHIAPTEGSSLTISIGLSTMTPQQGTDCRQVIVAADKGLYTAKHNGRNQVGIE

SEQ ID NO: 13 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttgccgcgctgaaaattccgcacattgcgccgactgaggggtcgagtctgaccatcagtattgggctgtcgaccatgacgccgcagcaggggacggattgtcggcaggtgatagtggcggcggataaggggttgtatacggctaagcataatgggcgcaatcaggtggggattgagtag

SEQ ID NO: 14 - protein, 333 aa, Pseudomonas fluorescens
MNDLQIDDIKTDENAAMVLLVDDQAMIGEAVRRGLAHEENIDFHFCADPHQAIAQAIRIKPTVILQDLVMPGLDGLTLVREYRNHPATQNIPIIVLSTKEDPLIKSAAFSAGANDYLVKLPDNIELVARIRYHSRSYMTLLQRDAAYRALRVSQQQLLDTNLVLQRLMNSDGLTGLSNRRHFDEYLELEWRRAMRDQTQLSLLMIDVDFFKTYNDSFGHVEGDEALRKVAATIREASSRPSDLPARYGGEESALVLPNTSPGGARLVAEKLRMAVAALKIPHIAPTEGSSLTISIGLSTMTPQQGTDCRQVIVAADKGLYTAKHNGRNQVGIE

SEQ ID NO: 15 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtctgccctggtgctgcctaatacctcgccgggcggggcgcggttggtggctgagaagctgcggatggcggttgccgcgctgaaaattccgcacattgcgccgactgaggggtcgagtctgaccatcagtattgggctgtcgaccatgacgccgcagcaggggacggattgtcggcaggtgatagtggcggcggataaggggttgtatacggctaagcataatgggcgcaatcaggtggggattgagtag

SEQ ID NO: 16 - protein, 333 aa, Pseudomonas fluorescens; identical to SEQ ID NO: 12

SEQ ID NO: 17 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttgccgcgctgaaaattccgcacattgcgccgactgaggggtcgagtctgaccatcagtattaggctgtcgaccatgacgccgcagcaggggacggattgtcggcaggtgatagtggcggcggataaggggttgtatacggctaagcataatgggcgcaatcaggtggggattgagtag

SEQ ID NO: 18 - protein, 333 aa, Pseudomonas fluorescens
MNDLQIDDIKTDENAAMVLLVDDQAMIGEAVRRGLAHEENIDFHFCADPHQAIAQAIRIKPTVILQDLVMPGLDGLTLVREYRNHPATQNIPIIVLSTKEDPLIKSAAFSAGANDYLVKLPDNIELVARIRYHSRSYMTLLQRDAAYRALRVSQQQLLDTNLVLQRLMNSDGLTGLSNRRHFDEYLELEWRRAMRDQTQLSLLMIDVDFFKTYNDSFGHVEGDEALRKVAATIREASSRPSDLPARYGGEEFALVLPNTSPGGARLVAEKLRMAVSALKIPHIAPTEGSSLTISIGLSTMTPQQGTDCRQVIVAADKGLCTAKHNGRNQVGIE

SEQ ID NO: 19 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttaccgcgctgaaaattccgcacattgcgccgactgaggggtcgagtctgaccatcagtattgggctgtcgaccatgacgccgcagcaggggacggattgtcggcaggtgatagtggcggcggataaggggttgtgtacggctaagcataatgggcgcaatcaggtggggattgagtag

SEQ ID NO: 20 - protein, 333 aa, Pseudomonas fluorescens
MNDLQIDDIKTDENAAMVLLVDDQAMIGEAVRRGLAHEENIDFHFCADPHQAIAQAIRIKPTVILQDLVMPGLDGLTLVREYRNHPATQNIPIIVLSTKEDPLIKSAAFSAGANDYLVKLPDNIELVARIRYHSRSYMTLLQRDAAYRALRVSQQQLLGTNLVLQRLMNSDGLTGLSNRRHFDEYLELEWRRAMRDQTQLSLLMIDVDFFKTYNDSFGHVEGDEALRKVAATIREASSRPSDLPARYGGEEFALVLPNTSPGGARLVAEKLRMAVAALKIPHIAPTEGSSLTISIGLSTMTPQQGTDCRQVIVAADKGLYTAKHNGRNQVGIE

SEQ ID NO: 21 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttgggcaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttgccgcgctgaaaattccgcacattgcgccgactgaggggtcgagtctgaccatcagtattgggctgtcgaccatgacgccgcagcaggggacggattgtcggcaggtgatagtggcggcggataaggggttgtatacggctaagcataatgggcgcaatcaggtggggattgagtag

SEQ ID NO: 22 - protein, 333 aa, Pseudomonas fluorescens
MNDLQIDDIKTDENAAMVLLVDDQAMIGEAVRRGLAHEENIDFHFCADPHQAIAQAIRIKPTVILQDLVMPGLDGLTLVREYRNHPATQNIPIIVLSTKEDPLIKSAAFSAGANDYLVKLPDNIELVACIRYHSRSYMTLLQRDAAYRALRVSQQQLLDTNLVLQRLMNSDGLTGLSNRRHFDEYLELEWRRAMRDQTQLSLLMIDVDFFKTYNDSFGHVEGDEALRKVAATIREASSRPSDLPARYGGEEFALVLPNTSPGGARLVAEKLRMAVAALKIPHIAPTEGSSLTISIGLSTMTPQQGTDCRQVIVAADKGLYTAKHNGRNQVGIE

SEQ ID NO: 23 - DNA, 1002 bp, Pseudomonas fluorescens
atgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgtgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttgccgcgctgaaaattccgcacattgcgccgactgaggggtcgagtctgaccatcagtattgggctgtcgaccatgacgccgcagcaggggacggattgtcggcaggtgatagtggcggcggataaggggttgtatacggctaagcataatgggcgcaatcaggtggggattgagtag

SEQ ID NO: 24 - DNA, 1136 bp, Pseudomonas fluorescens
  misc_feature (174)..(174): n = a, c, g or t/u
ggtacctggctcagggtggctcggaagccgagttgtatcgtcacttcatcgccgtatccagcaacaacgcggcagccgtggccttcggtatccgcgaagaaaacatcttcccgatgtgggactgggtcggcggccgttactcactgtggtcggccatcggcctgtctctccatnggcttgcccatcgccctggcgatcggcatgtccaacttcaaggaactgctgtccaggtgcctacaccatggaccagcatttccagaacgccccattcgaagccaacaagccggtgctgctgggcttgctgggcgtgtggtacggcaacttctggggtgcgcagagccacgcgatcctgccgtacgaccactacctgcgtaacatcaccaagcacttgcaacagctggacatggaatccaacggcaagagcgttcgccaggacggtacgcccgtcgctaccgataccggcccggtgatctggggtggcgtgggttgcaacggccagcacgcttaccaccagttgctgcatcaggggaccaactgatcccggccgacttgcatcgtgccgatcgtcagcttcaacccggtgtccgaccaccatcagtggctgtacgccaactgcctgtcccagagccaggcgctgatnctcggcaagacccgcgtcgaagccgaagccgagctgcgcgacaaaggcatcccggaagacgaagtgcagaagctggcaccgcacaaggtgatcccgggcaaccgtccgagcaacaccctcgtggtcgagcgcatcagcccgcgtcgctggggcactggtcgccatgtatgagcacaaagtgttcgtgcagagcgtgatctggggcatcaacgccttcgaccaatggggcgtggaactgggcaaagagctgggcaaaggcgtctacaaccgcctgaccggcgccgaagaaacctcggccgaggacgcttcgacccagggcctgatcaactacttccgcggtcgtcaccgcggctgatacaggtattgcagggccaacgtccgtttggaggttggccttgaacccttcctttgcatggtgcatctntatgacttgtggctaaaacaagaataaggatcgctcatgttcgatatcagcacgtttcccaccgccgatgcc

SEQ ID NO: 25 - DNA, 703 bp, Pseudomonas fluorescens
  misc_feature (5)..( 5): n = a, c, g or t/u
gtcangtcatggaatctcagtgaaacagacgagctgtcgtgtgtagatgtcngaacactacgcattctacggtcttacggncgcnnttgtgggggttggtgcacagaaagtcggccaatttagcacattggcgccttgcccaaaatccctgtcgttgttagagtttgccgcacttttttacccaggttgcctagggtccctttcccatgttcaagaaactggtggatgttttccagcgatctttccattgacctggcactgccaacacccttatttacgtgcgagcgcggtatcgtcctgaatgagccatcggttgtggccattcggaccatggtaatcagaaaagtgtcgttncgtcggcaccgaggccaagcgcatgctcggccgtacaccaggcaatattgctgccattcgtccgatgaaagacggcgtgatcgccgacttcagtgtctgcgagaagatgctgcagtacttcatcaacaaggttcacgaaaacagtttcctgcagcccagccctcgtgtgctgatctgcgttccatgcaaatccacccaggttgagcgtcgtgccatccgtgaatcggcccttggtgccggtgctcgcgaagtgttcctgatcgaagagccgatggctgctgcgatcggtgccggcctgccggtagaagaagcccgcggttcgatggtggtggattcggtggtggtaccc

SEQ ID NO: 26 - DNA, 14000 bp, Escherichia coli
aacggaaagtcaaaaagtgagcaaattcccgtctcgccgtaaacaataaaccggcgaaatgctcacggttagaattgtctcatgaacggtacggttatttcatagggatcaagcaaaatgaataacaatgaaccagatactctgcctgatcccgcgataggctatatcttccagaatgatattgtggcgttaaagcaggcattttcactgcctgatattgattatgccgatatttcccaacgcgaacagttggccgcggcattaaaacgctggccgttgctggcagagtttgcgcaacaaaagtaggggattggtgaatggccgtactgggatagcagggggtgcggggaggcgtggggacaacaaccatcaccgccgcattagcctggtcattacaaatgttgggagaaaatgtcctggtggtcgatgcctgcccggacaacttgttgcgcctgtcatttaatgttgattttacccaccgtcagggctgggccagagcgatgctggatggccaggactggcgtgacgctgggttgcgctacacctcgcagctcgatttgctgccttttggtcagttatccattgaagaacaagaaaatccacagcactggcaaacccggctgagcgatatttgctccggcttacagcaactaaaagccagcgggcgttaccagtggattttaatcgacttaccgcgtgatgcctcgcagataacccaccagctgctgagtttgtgcgatcactcgctggcaatcgtcaatgtggatgccaactgccatatccgactgcatcagcaagcgctgccggatggcgcacatattttgattaatgacttccgtattggcagtcaggttcaggacgatatttaccagctttggttgcaaagccagcgccgattactgccgatgctcattcatcgtgatgaagcgatggctgaatgcctggcggctaagcaaccagtaggtgaatatcgcagtgatgcgctggcggctgaagagatactgacgctggcgaactggtgcctgttgaactactccgggctgaaaacgccagtcgggagtgcatcatgagtatcctgacccggtggttgcttatcccgccggtcaacgcgcggcttatcgggcgttatcgcgattatcgtcgtcacggtgcgtcggctttcagcgcgacgctcggctgtttctggatgatcctggcctggatttttattccgctggagcacccgcgctggcagcgtattcgcgcagaacataaaaacctgtatccgcatatcaacgcctcgcgtccgcgtccgctggacccggtccgttatctcattcaaacatgctggttattgatcggtgcatcgcgcaaagaaacgccgaaaccgcgcaggcgggcattttcaggtctgcaaaatattcgtggacgttaccatcaatggatgaacgagctgcctgagcgcgttagccataaaacacagcatctggatgagaaaaaagagctcggtcatttgagtgccggggcgcggcggttgatcctcggtatcatcgtcaccttctcgctgattctggcgttaatctgcgttactcagccgtttaacccgctggcgcagtttatcttcctgatgctgctgtggggggtagcgctgatcgtacggcggatgccggggcgcttctcggcgctaatgttgattgtgctgtcgctgaccgtttcttgccgttatatctggtggcgttacacctctacgctgaactgggacgatccggtcagcctggtgtgcgggcttattctgctcttcgctgaaacgtacgcgtggattgtgctggtgctcggctacttccaggtagtatggccgctgaatcgtcagccggtgccattgccgaaagatatgtcgctgtggccgtcggtggatatctttgtcccgacttacaacgaagatctcaacgtggtgaaaaataccatttacgcctcgctgggtatcgactggccgaaagataagctgaatatctggatccttgatgacggcggcagggaagagtttcgccagtttgcgcaaaacgtgggggtgaaatatatcgcccgcaccactcatgaacatgcgaaagcaggcaacatcaacaatgcgctgaaatatgccaaaggcgagttcgtgtcgattttcgactgcgaccacgtaccaacgcgatcgttcttgcaaatgaccatgggctggttcctgaaagaaaaacagctggcgatgatgcagacgccgcaccacttcttctcaccggacccgtttgaacgcaacctggggcgtttccgtaaaacgccgaacgaaggcacgctgttctatggtctggtgcaggatggcaacgatatgtgggacgccactttcttctgcggttcctgtgcggtgattcgtcgtaagccgctggatgaaattggcggcattgctgtcgaaaccgtgactgaagatgcgcatacttctctgcggttgcaccgtcgtggctatacctccgcgtatatgcgtattccgcaggcggcggggctggcgaccgaaagtctgtcggcgcatatcggtcagcgtattcgctgggcgcgcgggatggtacaaatcttccgtctcgataacccgctcaccggtaaagggctgaagtttgctcagcggctatgttacgtcaacgccatgttccacttcttgtcgggcattccacggctgatcttcctgactgcgccgctggcgttcctgctgcttcatgcctacatcatctatgcgccagcgttgatgatcgccctattcgtgctgccgcatatgatccatgccagcctgaccaactccaagatccagggcaaatatcgccactctttctggagtgaaatctacgaaacggtgctggcgtggtatatcgcaccaccgacgctggtggcgctgattaacccgcacaaaggcaaatttaacgtcaccgccaaaggtggactggtggaagaagagtacgtcgactgggtgatctcgcggccctacatcttccttgtcctgctcaacctggtgggcgttgcggtaggcatctggcgctacttctatggcccgccaaccgagatgctcaccgtggtcgtcagtatggtgtgggtgttctacaacctgattgttcttggcggcgcagttgcggtatcggtagaaagcaaacaggtacgccgatcgcaccgcgtggagatgacgatgcccgcggcaattgcccgcgaagatggtcacctcttctcgtgtaccgttcaggatttctccgacggtggtttggggatcaagatcaacggtcaggcgcagattctggaagggcagaaagtgaatctgttgcttaaacgcggtcagcaggaatacgtcttcccgacccaggtggcgcgcgtgatgggtaatgaagttgggctgaaattaatgccgctcaccacccagcaacatatcgattttgtgcagtgtacgtttgcccgtgcggatacatgggcgctctggcaggacagctacccggaagataagccgctggaaagtctgctggatattctgaagctcggcttccgtggctaccgccatctggcggagtttgcgccttcttcggtgaagggcatattccgtgtgctgacttctctggtttcctgggttgtatcgtttattccgcgccgcccggagcggagcgaaacggcacaaccatcggatcaggctttggctcaacaatgatgataacgcgatgaaaagaaaactattctggatttgtgcagtggctatggggatgagtgcgttcccctctttcatgacgcaggcgacgccagcaacgcaaccactgatcaatgctgagccagctgtagccgcccagacggaacaaaatccgcaggtggggcaagtgatgccgggcgtgcagggcgctgatgcgccagtcgtggcgcagaacggtccttcgcgtgatgtgaagctgacctttgcgcaaattgcaccgccgccgggcagcatggtgctacgtggcattaacccgaacggcagcattgagtttggtatgcgcagcgatgaagtggtgacgaaggcgatgctcaacctcgaatacaccccatcgccatcgttactgcctgtccagtcgcagttaaaggtttatctcaatgatgaactgatgggcgtgctgccagtgaccaaagaacagttgggtaaaaaaacgctggcgcaaatgcccattaacccactgtttattagcgacttcaaccgtgtacggctggagtttgtcggccattatcaggacgtgtgcgaaaaaccggccagcaccacgctttggctggatgttgggcggagcagtggactggatctgacctatcagaccctgaatgtgaagaatgacctgtcacacttcccggtgccattctttgacccgagcgataaccgcaccaacaccttgccgatggtctttgcgggtgcgccggatgttgggctgcaacaagcctctgccattgtcgcctcgtggtttggttcgcgttctggctggcgtgggcagaacttcccggtactctataaccaactgccggatcgcaatgccattgtctttgcaaccaacgacaaacggccggacttcctgcgcgatcatccggcggtaaaagccccggtgattgagatgattaaccatccgcagaatccttacgtcaaactgctggtggtgtttggtcgtgacgacaaagacctgttgcaggcagcgaaaggtatcgctcagggtaacattctgttccgtggtgaaagcgtggtagtgaatgaagtgaaaccgctgctaccgcgtaagccgtacgatgcgccgaactgggtacgtaccgatcgtccggtcacatttggcgaactgaaaacctatgaagaacagttacaatccagcggtcttgagccagcagcgattaacgtttcgctaaacctgccgccggatctctacctgatgcgcagtaccggcattgatatggatattaattaccgctacaccatgccgccggtgaaagacagttcgcggatggatatcagcctgaataaccagttcctgcaatccttcaacctgagcagcaaacaggaggcgaaccgcctgctgctgcggattccggtattacaaggtttgctggatggcaaaacagatgtctctattccggcgctgaaactgggcgcgaccaaccagctgcgcttcgactttgagtatatgaacccgatgccgggcggttcggtggataactgtattaccttccagccggtgcagaatcatgtggtgattggtgacgactccaccatcgacttctcgaagtattaccacttcatcccgatgccggatctacgcgcctttgctaacgcgggcttcccattcagccggatggcggatctgtcgcaaaccatcaccgtgatgccgaaagcgcctaacgaagcacagatggaaacgttgctgaatactgttggttttatcggcgcacagacgggcttcccggcgattaatctgacggtgaccgatgatggcagcaccattcagggcaaagatgccgacatcatgatcatcggtggtatcccggacaaactgaaagacgataagcagatcgacctattggtgcaggcgaccgaaagctgggtgaaaacaccgatgcgccagaccccgttccccggcattgtgccggacgagagcgatcgcgcggcagaaacccggtcaacgctgacctcttccggtgcgatggcggcggtgattggcttccagtcgccgtataacgaccagcgcagcgtgattgcgctgttggcagatagcccacgcggttatgaaatgcttaacgatgcggtgaacgatagcggcaaacgcgccaccatgttcggttcggtcgcggtgatccgcgagtccggtatcaacagcctacgtgttggcgacgtttattacgtaggtcatctgccgtggttcgagcgcgtgtggtatgcgctggcaaaccatccgattctgctggcggtgctggcggctatcagtgtgatattgctggcatgggtactgtggcgtctgctgcgaattattagtcgtcgtcgtcttaacccggataacgagtaattgaagatgaatgtgttgcgtagtggaatcgtgacgatgctgctgctggctgcctttagtgttcaggcagcctgtacctggcctgcctgggagcagtttaaaaaggattacatcagtcaggaagggcgcgtcatcgaccccagcgacgcgcgcaaaatcaccacctccgaagggcaaagttacggcatgttctctgccctggcggctaacgaccgtgcagctttcgataatattctcgactggacgcagaacaatctcgctcagggttctttaaaagaacgtttgcccgcctggctgtggggcaagaaagagaacagtaagtgggaagtgctggacagcaattcggcctccgatggtgatgtctggatggcctggtcgttgctggaggcggggcgtttgtggaaagagcagcgttataccgacatcggcagcgcgttgctaaaacgtatcgcgcgggaggaagtggtgacggtgcctgggctgggttccatgttgttaccgggcaaagtgggttttgctgaggataacagctggcgttttaaccccagctacctgccgccgacgctggcgcagtatttcacccgctttggcgcgccgtggaccacgctgcgcgaaaccaatcaacgtttattgctggaaaccgccccgaaaggtttttcgccagactgggtgcgctatgagaaagacaaaggctggcagctaaaagccgaaaaaacattgatcagcagctacgacgctatccgcgtttacatgtgggtaggcatgatgcctgacagcgatccgcaaaaagcgcggatgctcaaccggtttaaaccgatggcgacattcactgagaaaaacggttatccgccggaaaaagtggatgtggctacggggaaagcgcagggtaaaggaccagtcggtttttctgccgccatgctgccctttttacaaaaccgcgatgcgcaggccgttcagcgccagcgcgtggccgataactttcccggcagcgatgcctattacaactatgtgctgaccctgtttggacaaggctgggatcaacaccgtttccgcttctcgacaaaaggtgagttattacctgactggggccaggaatgcgcaaattcacactaaacatattccgctttccctcggtctggccgtcatgccgatggtcgaggcagcaccaaccgctcagcaacagttgctggagcaagttcggttaggcgaagcgacccatcgtgaagatctggtgcaacagtcgttatatcggctggaacttattgatccgaataacccggacgtcgttgccgcccgtttccgttctttgttacgtcagggcgatattgatggcgcgcaaaaacagctcgatcggctgtcgcagttagcgccgagttcaaatgcgtataaatcgtcgcggactacgatgctactttccacgccggatggtcgtcaggcactgcaacaggcacgattgcaggcgacgaccggtcatgcagaagaagctgtggcgagttacaacaaactgttcaacggtgcgccgccggaaggtgacattgctgtcgagtactggagtacggtggcgaaaattccggctcgccgtggcgaagcgattaatcagttaaaacgcatcaatgcggatgcaccgggcaatacgggcctgcaaaacaatctggcgctattgctgtttagtagcgatcgccgtgacgaaggttttgccgtcctggaacagatggcaaaatcgaacgccgggcgcgaaggggcctctaaaatctggtacgggcagattaaagacatgcccgtcagtgatgccagtgtgtcggcgctgaaaaaatatctctcgatctttagtgatggcgatagcgtggcggctgcgcaatcgcaactggcagaacagcaaaaacagctggccgatcctgctttccgcgctcgtgcgcaaggtttagcggcggtggactctggtatggcgggtaaagccattcccgaactacaacaggcggtgcgggcgaacccgaaagacagtgaagctctgggggcgctgggccaggcgtattctcagaaaggcgatcgcgccaatgcagtggcgaatctggaaaaagccctcgcactggacccgcacagcagcaacaacgacaaatggaacagtctgctgaaagtaaaccgctactggctggcgatccagcagggcgatgctgcgctgaaagccaataatcctgaccgggcagaacgcctgttccagcaggcgcgtaatgtcgataacaccgacagttatgcagtgctggggctgggcgatgtggcgatggcgcgaaaagattatcccgccgccgaacgttattatcagcagaccttgcgtatggacagcggcaacactaacgccgtgcgcgggctggcaaatatttaccgccagcaatcgccagaaaaagctgaagcgtttatcgcctcgctctctgccagtcagcggcgtagcattgatgatatcgaacgcagcctgcaaaacgaccgtctggcacagcaggcagaggcactggaaaaccagggcaaatgggcgcaggcggcagcacttcagcggcaacgactggcgctggaccccggcagcgtatggattacttaccgactttcgcaggatctctggcaggccggacaacgcagccaggccgatacgttaatgcgcaatctggcgcagcagaagtcgaacgacccggagcaggtttacgcttacgggctgtacctctctggtcatgaccaggacagagcggcgctggcgcatatcaatagcctgccgcgtgcgcagtggaacagcaatattcaggagctggttaatcgactgcaaagcgatcaggtgctggaaaccgctaaccgcctgcgagaaagcggcaaagaggcagaagcggaagcgatgctgcgccagcaaccaccttccacgcgtattgacctcacgctggctgactgggcgcaacaacgacgtgattacaccgccgcccgcgctgcatatcagaatgtcctgacgcgggagccagctaacgccgacgccattcttggtctgacggaagtggatattgctgccggtgacaaagcggcggcacgtagccagctggcgaaactgcccgctaccgataacgcctcgctgaacacacagcggcgcgtggcgctggcacaggcgcagcttggcgataccgcagcagcgcagcggacgtttaataagttgatcccgcaggcaaaatctcagccaccgtcgatggaaagcgcgatggtgctgcgtgatggtgcgaagtttgaagcgcaggcgggcgatccaacgcaggcgctggaaacctacaaagacgccatggtcgcatccggtgtgactacgacgcgtccgcaggataacgacacctttacccgactgacccgtaacgacgagaaagatgactggctgaaacgtggcgtgcgcagcgatgcggcggacctctatcgccagcaggatcttaacgtcacccttgagcacgattactggggttcgagcggcaccggtggttactccgatctgaaagcgcacactaccatgttgcaggtggatgcgccgtattctgacgggcggatgttctttcgcagtgatttcgtcaatatgaacgtcggcagtttctccactaatgccgatggcaaatgggatgacaactggggcacctgtacattacaggactgtagcggcaaccgcagccagtcggattccggtgccagcgtggcggtcggctggcgaaatgacgtctggagctgggatatcggtaccacgccgatgggcttcaacgtggtggatgtggtcggcggcatcagttacagcgatgatatcgggccgctgggttacaccgttaacgcccaccgtcggcccatctccagttctttgctggcctttggtgggcaaaaagactccccgagcaataccgggaaaaaatggggtggcgtacgtgccgacggtgtggggctaagtctgagctacgataaaggtgaagcaaacggcgtctgggcatcgcttagtggcgaccagttaaccggtaaaaatgtcgaagataactggcgcgtgcgctggatgacgggctattactataaggtcattaaccagaacaatcgccgcgtcacaatcggcctgaacaacatgatctggcattacgacaaagatctgagtggctactcactcggtcagggcggttactacagtccgcaggaatacctgtcgtttgccataccggtgatgtggcgggagcgcacggaaaactggtcgtgggagctgggtgcgtctggctcgtggtcgcattcacgcaccaaaaccatgccgcgttatccgctgatgaatctgatcccgaccgactggcaggaagaagctgcgcggcaatccaacgatggcggcagcagtcagggcttcggctacacggcgcgggcattacttgaacgacgtgttacttccaactggtttgttggcacggcaattgatatccagcaggcgaaagattacgcacccagccatttcctgctctacgtacgttattccgccgccggatggcagggtgacatggatttaccgccgcagccgctgataccttacgccgactggtaagttttcagatagcgcctctcttaatgccgctgcgatcgggtatactcgggcggcaatctgggatttccggggggagacaatttgcgcgtaagtcgctcgttaacaatcaagcagatggcaatggtggcagccgttgtcctggtgttcgtttttattttttgcaccgttttgctgttccatctggtccagcagaatcgctataacacggctacgcaactggaaagcattgctcgctctgtccgcgaacccttatcttcagctattttgaaaggcgatattcccgaagcggaagctattcttgccagcattaaaccggcaggcgtggtcagccgtgccgatgtagtgctgcctaaccagttccaggcgctgcgtaaaagttttattccagagcgcccggtgccggtaatggttactcgcctgtttgagctaccggttcaaatctcgctgggcgtttactcgctcgaacgtccggcaaacccgcagccaattgcctatctggtactacaggcggattccttccgtatgtataagttcgtgatgagcaccctctcaacgttagtgaccatttacttacttttgtcgcttatcctgaccgtcgccatcagctggtgcattaaccgcctgattttgcatccgttacgcaatattgctcgcgaacttaacgccatcccagccaaggagcttgttggtcaccaactggcattaccgcgtctgcatcaggacgatgaaatcggtatgttggtgcgcagttacaacctcaaccagcaattgctgcagcgccattatgaagaacagaacgaaaatgcgatgcgcttcccggtgtcggatttgccgaacaaagccttgctgatggagatgctggagcaggttgtcgcgcgtaaacaaaccaccgcgctgatgatcatcacctgtgaaaccctgcgtgatactgcgggcgtgctgaaagaggcgcaacgagaaattctgctgctgacgctggtggaaaaactcaaatcggtactgtcgccacgtatgatcctcgcgcagattagcggttatgactttgctgtcattgccaacggtgtacaggaaccgtggcacgcaatcaccttaggtcagcaagtgctcactatcatgagcgagcgcctgccgattgaacgtattcaactccgtccgcactgtagcattggcgtggcgatgttctacggcgatctcaccgccgaacagctttacagtcgcgctatttctgcggcatttaccgctcgccataaaggcaagaatcagattcagttctttgatccgcagcagatggaagccgcccagaagcggttgacggaagagagcgatatccttaatgcactggaaaatcatcagtttgctatttggttacagccacaggtcgagatgaccagcggtaaactggtcagtgcggaagtgttactgcgtatccagcaaccggatggcagttgggacctgccggatggcttaatcgatcgcattgagtgctgtgggctgatggttaccgtcggtcactgggtgctggaagagtcctgtcgattgcttgcagcctggcaagagcgcggcattatgctgcccttgtcggtaaacctctctgcgctgcaactgatgcacccgaatatggtggcggatatgctggaactgttaacccgctatcgcattcagccgggaacactgattctggaagtgacagaaagccgacgtattgacgaccctcatgctgcggtggcaatcctccgtccgctgcgcaatgccggagttcgggtggcgctggatgatttcggcatgggctacgcagggctgcgtcagctgcagcatatgaaatcgttgccaatcgacgtactgaaaatcgacaaaatgtttgttgaaggcttgccgggagatagcagcatgattgctgcaattatcatgctggcgcagagcctgaacttacaaatgattgccgaaggcgtggagactgaagcacaacgcgactggctggcaaaagcgggcgttggtattgcccagggcttcctttttgctcgcccactccctattgaaatcttcgaagagagttacctggaagaaaagtagctaccccaaactgattacaaaactttaaaaagtgctggtttgtgcgagccagctcaaactttttaacctttttgtttcaattatgatccaggtacatttctgtgatgttgtctgggtgttattttaaggccgcaggtaccccataaccttacaagacctgtggttttactaaaggacaccctatgaaaacctctctgtttaaaagcctttactttcaggtcctgacagcgatagccattggtattctccttggccatttctatcctgaaataggcgagcaaatgaaaccgcttggcgacggcttcgttaagctcattaagatgatcatcgctcctgtcatcttttgtaccgtcgtaacgggcattgcgggcatggaaagcatgaaggcggtcggtcgtaccggcgcagtcgcactgctttactttgaaattgtcagtaccatcgcgctgattattggtcttatcatcgttaacgtcgtgcagcctggtgccggaatgaacgtcgatccggcaacgcttgatgcgaaagcggtagcggtttacgccgatcaggcgaaagaccagggcattgtcgccttcattatggatgtcatcccggcgagcgtcattggcgcatttgccagcggtaacattctgcaggtgctgctgtttgccgtactgtttggttttgcgctccaccgtctgggcagcaaaggccaactgatttttaacgtcatcgaaagtttctcgcaggtcatcttcggcatcatcaatatgatcatgcgtctggcacctattggtgcgttcggggcaatggcgtttaccatcggtaaatacggcgtcggcacactggtgcaactggggcagctgattatctgtttctacattacctgtatcctgtttgtggtgctggtattgggttcaatcgctaaagcgactggtttcagtatcttcaaatttatccgctacatccgtgaagaactgctgattgtactggggacttcatcttccgagtcggcgctgccgcgtatgctcgacaagatggagaaactcggctgccgtaaatcggtggtggggctggtcatcccgacaggctactcgtttaaccttgatggcacatcgatatacctgacaatggcggcggtgtttatcgcccaggccactaacagtcagatggatatcgtccaccaaatcacgctgttaatcgtgttgctgctttcttctaaaggggcggcaggggtaacgggtagtggctttatcgtgctggcggcgacgctctctgcggtgggccatttgccggtagcgggtctggcgctgatcctcggtatcgaccgctttatgtcagaagct

SEQ ID NO: 27 - DNA, 13288 bp, Pseudomonas fluorescens
atatgacgagtctagagggtaagttcaattatcattttagcggttggttaacatttatcgaaaagtgatctaatgatcatagtatggccgttagtaaattcatgatggaaagtatacgtcagtaatcgcccgtgtatcacgatagccaagcatctaagcgcgcgtattgtgtgtttgcctgacgaaatactggtacatctcacggcttgcgtgtcttgactcttgaagaaggatcttcattcagatttcgcacttcaggttggcgtttttgtcaggccgctttgatcgatgcaacttttccgggctgtctgcgtcgacatggaccactgattggaaaccgggtgtcttacgtgcattctccagcatcaggctgagccgatcatccggaatcatggcgccagcccatatgtaattgatttccttcttgatcggcgtaacggccgtgggaacgggggcaagggctgcgtgaactgtttcaaaggtggttggcttgggcagtgtcgccttgacccgcaagaactcgtccaggctgccacttttccactcgtaggggcctgttcgccaaaaaggggcgcccagggttttaccgtcgcctagcttgcgccaaacgtcattggctacatcaaactctactaaaacgcaaaaattggcataaagccttgtcttgccatcatcactaagtacgcggttgacttcgtccattgggacttcatagcttttataaggcattccctcgacaccgcccggcagccccaggcgttgccattgatcggcgccggttttgtttaatatgaattcagtttgtcggtaggtcttggggttgatgacacgaacctgcaccgaactcatggggctaccgcccgaagcttctcgaacctgatagacagaagccttgccagacttgtcgacgtggcggatgtattgactctggccatcgaggctgtggtaaaccccttggctatccgggctcaggccgacgaggcgcgagggtgaaactgtgtatttttcgaccactgcgagttcacccggcaggccgggtggatgcttcaacttgaacgcgcgagcagccgttgaaagcatttggcgcgtgccttgacccaaaccctcggccagcgcgccaaggccgtccgctgggttaagagcgccgactgccgcggcgccgattattttggccccggtgaacagttttgttccgagttttccagtgagcttggtcgcctggctgaccgccttgcccaccggaataacaaaacccagcaggtccagagcgaagtccccaacggccccccatacgtcgccttttactgccttttcaataccgcttttcaggggcaggaagtccatgacggcgcccagttcatccaccaactgtttttcttgctcttcaacagcattggttccactggcgtaggtcttgagcgcgtcgcggccatagaggacaaacttttcgatctcgttggccaggtgttgctcacgctcactggcccatggcgctttaggggaacggtcttgcccggtcttttctccccatgcctttggaaaagaaaaaggcaccggatcaatggaaaggcctgaagacacgacctcctttggcggcgcgccggttttgtaggcgtccaagtcaatgtcgagcgacttgcctttggccttttcaagaaagtagtcatccgggtcatcgtagccacgtaggggcgtttgatgaacgctgctgccccatagctgatggttgcggcgaacgagcccctgcatcggcaacacttcgtaaatccgcacgtcacctttgttttttacacacatcagtagaccgtgacgtgctctggacgcgtcgacttgagcgggtgtctcagtgtcgccgttggcttcgcgcacagagtggaaggtgatgtcgccgaccgcgatatgttggcgttcatcaagaggcagttgggaaaactggtattgcatcgcggtgtttatgcctgtcctcaatcccttgatgtaggcatcgaactgctcattgaaaatggccgtgatgtccggaaggggtctgacccccgtgcggcgggtcgggctcagtacgacgctctggtcaacgatacggtccatcttccctgccgtataaatgtctaccagcgcatgcttggtctcactgccgtcaaggttgacgatgggggtcagcggctgctcgaagtcatagtcgccatataccttcttcaattctgcgcaggccaatgccctacgcgtaggcagaaaggcggtgaggcctttattggcagtgcctaatgtttctagcaatgcgttgaatttgtctcgcgcaacggtgatctgttcgggcgtaaacgcatcatcgttagccttattcaggacgtcattggcgatggcccaatcgatgacagcatgtgtctgagcccccgcctcaatgccagcatccttaagcgtgaccgggcttctgttgccaaaactcatgacttggccaaacgtcatgttacgggtggcgcccggctccagcgcctcaatgcgcgcgacagcggtgctcaggctggcccaggtgtgggacccatacaccaagccgccagggaggtccttgaccaggaacgcaggtgctgcactggcgagcaatagctgtgccgccgcaggggccaggtcgtgatcgactcggccttgctgcttaagatgctcgacgagtcgcgctgttataacggcaggtgacaagccccggtgagctgcctgggccaggtcgtaaccggcgacgacgttgcgttgggtgcccacggcgggatccaggtcgagcgccagcgcggtcatcgcccagtcgctggctgtgctggttgaccaggtgtccgcgaacgcgcttcgcaggcgcttacccaacgcctgtgcctgcggactagcaatcagagcctgcaggcgttgcgcggggctatcagcgttggtgagatcgggagtttttttagccaggtggttgagaaggcaggtgcccccctccaacccatttcgtgcctcttcactgacgatgttctggacgcgttcgcgctcgtcacgtccgagttggcgcgtgtccgacagcagtccccagtgcctcgagttgtcaggcggttgtggcaacgggtctgacagaacggcaataaggttcgacagctcggccttgtcctggggaatcatcaagccgtgcgcatgcaaaaacggctcaagcttcaaaccgctttccggcatgcctgggtaagtcttcgcgaatacggaatccgggtggacggggacacggtgcgagtcgatgtgttcggccagcgtcgtgtttgaattcgttgaggccagcacactcatacgggtgagcgtatggataaggcttaggcggttgcgtctgtcacccaggttttgtttctgaagctcgagggcttcaggggtgcgtgtatggggaggtcttaacccatccgatgggtcgagtggtcggaaaccctggccttcattcaattgtgcaacacgcgttgccacctgggcccggctgaggttgtcgaggttttccccatagaaccgcccgataagctcaaagggcgcgctgtgtacaggtagcattgcgggattcctgatactgaaggtatcgggcgcgatcaccttgccggcgtcgagtatggggccggccaccgccgcccagcgcgcattgttgttgagggtaaaactgaccacgctctggtcggcttttcgcctggcgtgtaaggcgcctttggagggaatgatctcgacagtgctgctatcgagaccctccgaaagcatccaggcggtgaagtccgggctattcaacgtctcccgaaagtattgccaccattttccgaacgtcgagtcggggggaatgccggcaacaatcacctctcgttggctgtccccgccgcgcgcgcgtgccagcgcgtcgccgtagtaacctgccaggagcttgtccgggtaacccatcgctaaagcattgcctgcggcgatgctggctttcgctctatttgaaggcagcagactcaacggcaacggcgactttgctggcgtcttggcggtctcgggcacggaatctgtggaggctctgtcgctgcgatgctggctgaatgggttgccggtggtagggggggcgatccgtgtgtttggcgggggcgtaatggctagcggggtagagggggcggaaatattcaatggttatgctccttgcagggataggcgctgaagcgccaggcgtgggcaggcagttctacgccctcgtcgctgtctcgcagagtgcgtttgttttttcgggtgtttccgagcggctccagcctgcgtaaaacctgggtgcgtgggttgtatcttctattgtccactgtaaaaaaaagaacacggcttgacgttgcggaggtggggccccgccttccgatttcggattcgacccgtcctgatattcagcgaggagcacgtcagtggaggcggtgttgagggaggagggggcggcaatcaccccgcgaggattttttgcctgtggagcgttctatccgataacccattgataaacgatggcactgcctcagctatactcccctccatttgaatggcccttcgaggaattactgtgaagaactggaccttgcgccaacgcatcttggcaagttttgcggtcattatcgccatcatgctgttaatggtcgtggtctcctattcacggttgctgaagatcgaaaccagccaggaagccgtccgggacgacgcggtgccgggcgtttacctcagctcgatgatccgcagcgcctgggtcgacagctatttgcagaccatcgacatcatcggcctgcgcgacgacaaaaccttcaccaataccgataagaacgactacaagtcgttcgaagcgcgtattgaacagcagatggccaactacgaaaaaaccattcatggccaagctgaccgcatggagttcgataacttcaaggcggcgcacatcaactacaacaaagtgctggcccaggtgctggaacgcgttgaagccaatgacctgccgggcgcgaatcaattgctcgaggagcaattgacgccgatctggaccgaagggcgcatgaagctcaacgacatcattactgaaaacaagaacgtgtctgaccgggcgacggcggcgatcgacgaggcggtactgtcggccaagatcagcatggccgtgtcgctgctcatcgccatcttggctgccgggctgtgcggcctgttgctgatgcgcgcgatcatggcgccgatgcaacgcatcgtcgatatcctcgaaaccatgcgcgacggcgacctgagcaaacgccttaacctggagcgcaaggacgagttcggcgccgtcgaaaccggctttaacgacatgatgaccgagctcacggccctggtgtcccaggcccagcgctcgtcggtgcaggtcaccacctcggtgaccgaaattgccgccacgtccaagcagcaacaggccaccgcgactgaaacggccgcgacgaccactgagatcggtgctacgtcgcgcgaaatcgcggccacctccaaggacttggttcgcaccatgaccgaagtgtccaccgccgccgaccaggcgtcggtggctgccggctccggccagcaaggactggcgcgcatggaagagaccatgcactcggtgatgggcgcggccgacctggtcaacgccaagctggcgatcctcaatgagaaggccggcaacatcaaccaggtggtggtgaccatcgtcaaggtggccgaccagaccaacctgctgtcgctcaacgccgccatcgaagcggaaaaagccggcgagtacggtcgcggttttgccgtggtcgccaccgaagtacgccgcctcgcggaccagaccgccgtggctacctatgacatcgagcagatggtgcgcgagatccagtccgcagtgtctgcgggggtgatgggcatggacaagttctccgaagaagtgcgccgcggcatgttcgaagtgcagcaagtgggcgagcagttgtcgcagatcatccaccaggtacaggcgctggcgccgcgggtgttgatggtcaacgaaggcatgcaggcccaggccaccggcgccgagcagatcaaccacgccctcgtgcaactgggcgatgccagcagccagacggtggagtccctgcgccaggccagctttgccattgatgaactgagccaggttgcggtgggtctgcgcagcggcgtgtcgcgtttcaaagtctgatgagcgaactcgcggctaaacgcggcgccgtcccggcagcgaaaaaggcgttgttcctggtgttccatatcggtcaggaacgctatgccctcaaggctaccgaagtggccgaagtgctgccgcgcctgcctttgaaacccattgcccatgcaccgctgtgggtcgccgggatctttgcccatcgtggggcgctggtgccggtcattgacctcagcgccttgaccttcggcaacccggcccaggcccgcaccagcacgcggctggtgctggtcaattaccaacccgacgccgggtcccaagcgcgttggctgggactgatcctggagcaggccaccgacaccctgcgttgcgaccccgccgagttccagccctacggtctggccaaccgccaggcaccctacctggggccggtgcgcgaagatgcgctgggcctgatgcaatggatcggtgtaaacgacctgctgaccgatgacgtgcgcgccgtgctgttttctgccgagctgagcgtatgagcaacgacccgcgtttttttgcctttcttaaagagcgcatcggcctggacgtcgcgtcggtgggcgaagccatcatcgagcgcgccgtgcgccagcgcagccagatcgtccaggcacccacgccgggcgaatactggcagcacctgcaaagctcccaggacgaacagcaagcgctgatcgaagcagtgatcgtccccgaaacctggtttttccgttaccccgaatcctttgcgaccctggcgcgcctggcgaaggcccgcctggtcgatatcaagcagatgcgcgcattgcgtatcctcagcctgccgtgctccaccggcgaagaaccctattcgattgccatggcgctgctcgatgctggcctcgcgccgcatcagttcaaggtacaggggatggacgtcagcccgctgtcggtggagcgtgcgcgacgcggggtgtacggcaagaactcctttcgtggcggcgatattgccttccgtgaccgccacttcactgaatacggcgacggcttccacatcgccgatcgggtgcgcgaacaagtgcgcctgcaagtcggcaacctgcttgacccggcgctgctggttaatgaggccgcctatgacttcgtgttctgccgcaacctgctgatctacttcgaccagcccacccagaaacaagtcttcgacgtgctcaaaggcctcacccacgtggacggtgtgctgtttatcggcccggccgaaggcagcctgctggggcgccacggcatgcgttcgattggggtgccgcagtcctttgcgttcagccggcagggctcgccgcagctgcccgagccagcgtttataccgacgccggctcccacgcctccgcgcagcacggcgccgatttcagctaaaccacggccgttcagcaccgtcagcgcccacgttttaccgatcaaggctacgccctccgatgcaggcaccttgctcagccggatcgccaccctggccaacgaaggcaaaagtgccgaggcccgggccgcgtgtgaagactatttgaacagccacccgccggccgcccaggtgttttactggctggggctgctcagcgacgtggccggcagtgccctggaagcccagggctattatcgaaaagccttgtacctggaaccccagcacccgcaggccttgatgcacttggccgcgttgctcgagtcccggggcgacagtgcgggggcgcgccgtctgcaggcgcgagccgcccgtagcgagcgagccgacagtgagtccaaaccatgagtaacaccgaggcgctcgacaccaccggcctggacctgaccctggccgacactcaagccatcgacgactgctggaaccgcatcggtatccatggcgataagtcctgcccactactggctgaccatatccattgccgcaactgctcggtgtattccgccgccgccacgcgtctgctggaccgctacgccttgcagcaggacgaccgacgcccgcaggcggccgaagtcgacacggaggtggtcacccgttcgctcttgatgttccgcctcggcgaagaatggctgggcatcgctacacgctgcctggtggaagtcgcgccgttgcaaccgatccattccctgccgcatcaacgttcccgcgccttgctcggcgtggccaacgtgcgcggcgcgctggtggcgtgcttgtcgctggtggaactgctgggcctggacgccaccagcagcggcgccaccggcgggcgcatcatgccgcgcatgttgatcattgccgcccaggatggcccggtggtggtgccggtggatgaagtcgacggcatccatgccatcgatgagcgcaccttgaaggccgcatcggcgtccggcacccaggccagcgcgcgctttacccagggtgtattgccgtggaaaggccgcagcctgcgctggctggacgaggcgcaattgttgtccgccgtgacccggagcctctcatgacccccgaccagatgcgtgatgcctcgctgctggaattgttcagcctggaagccgatgcgcagacccaggtgttgagcgcgggcctgctcgccctggaacgcaacccgacccaggcggaccagctcgaagcctgcatgcgcgcggcgcactcgctcaagggcgccgcgcgcatcgttggggtggatgccggggtcagcgtgtcccacgtcatggaggattgcctggtcagcgcccaggaaaaccgcctgtacctgcaacccgaacatatcgatgcgctgctgcagggcactgatctgctgatgcgcatcgccacgccaggcaatgacgtcgggccggcggatgtcgaggcctacgtcgcgttgatggagcgtctgctggatccgtcccaggcgcctgtcaacgtcgcgccgtcgccggagccagcgcccgtcgtcgaagaactgccgccggaacccgaacccgcgccgccggtgaacagcgagccgccgcgtcaaggcaagcgcatgaccgaaggcggcgaacgcgtactgcgcgtcaccgccgagcgcttgaacagcctgctggacctgtcgagcaaatccctggtggaaacccagcggctcaagccgtacctggccagcctgcaacgcctcaagcgcattcaaagccaggggatacgggcgctggatacgctggacgggcaactcaagacccaggtcctgagcctcgaggcccaggaagccctggccgacacgcgccgtttgttgagcgaagcccaggccttgctggcggaaaagcacgccgagctggacgagttcggctggcaggccgggcagcgcgcccaagtgttgtatgacactgcactggcctgccgcatgcgcccgtttgccgatgtattggctggacaagtgcgcatggtgcgcgacctcggccgcagcctcggcaagcaagtgcgcctggagatcgagggcgaaaagacccaggtcgaccgtgacgtactggaaaaacttgaggcgccgctcacccatttgttacgcaatgccgtcgaccacggcatcgaaatgcctgagcaacgcctgctggcgggcaagccggctgaaggcttgatccgcctgcgggcctcccatcaggccggtttgctggtgctggaactcagtgatgacggcaatggcgtcgacctggagcgcctgcgcggcactatcgtcgatcggcacctgtcgccggtcgaaaccgccctgcgcctgagcgaagaagagttgctgacgttcctgttcctgccggggttcagcctgcgtgacacggtcaccgaagtgtccgggcgcggcgtgggcctggacgcggtgcagcacatggtccgccaactgcgcggcgcggtggtgctggagcagacggcggggcagggcagtcgtttccaccttgaggtgccgttgaccctgtcggtggtacgcagcctggtggtggaagtcggtgaggaagcctatgcgttcccgctggcgcatatcgaacgcatgtgtgacctcgcgcccgatgacatcgtgcaactggaaggtcgccagcatttctggcacgagggccggcatgttggcctggtcgccgccagccagttgttgcagcgcccggcggggcagagtccgtcagaaacgctgaaagtggtggtgatccgcgagcgcgatacggtgtacgggattgccgtggagcgctttatcggtgagcgtacgctggtggtgttgccgctcgatgatcgcctgggcaaggtccaggatatttccgccggtgccttgctggatgatggctcggtggtgttgatcgtcgacgttgaagacatgttgcgttcggtggacaaactgctgaacaccggccgattggaacgtattgcgcggcgcagccaacaaaccaccgaggcaccgcgtaagcgcgtgctggtggtcgatgactcgctgaccgtgcgtgagctgcaacgcaaattgctccttaatcgtggttatgaagtggccgtggcggtcgatggcatggacggctggaacgccttgcgctccgaagactttgacctgttgatcactgatattgatatgccccgcatggacggtattgaattggtcacactcttgcgccgtgacagtcgcctgcaatcgttgccggtgatggtggtgtcctacaaagatcgcgaagaagaccgacgtcgaggactcgacgccggtgcggactattatttagccaaagccagtttccacgatgacgccctgctggacgccgtggtggaactgatcggaggcgcacgggcatgaggattgcgatcgtcaatgacatgcccctggcagtggaggccttgcgccgcgccttgagcttcgagcctgcgcaccaagtggtgtgggtggccagcaatgggctggaagcggtgcaacgctgcgccgaactgacgccggacctgatcctgatggacctgatcatgccggtgatggacggcgtggaagccactcgccagatcatggccgagacgccgtgcgccatcgttatcgtgaccgtcgaccgccaggccaacgtgagccgggtgttcgaggccatgggccacggcgccctggacgtggtggacaccccgccgctcggcgtgggcaaccccaaggatgcggcggcgccgttgctgcgcaagatcctcaatatcggctggctgatcggccagcgcggcacccgcgtgcgcgccgaaaccctgccggcgcgcgcatccggcaaacgtcaaagcctggtggctatcggctcctcggcgggtggtccggctgccctggaaatcctgctcaagggtttacctcgcgactttccagccgccatcgtgctggtgcagcatgtggaccaagtgttcgcggcgggcatggccgagtggctgagcagcgcctcgggcctgccggtacgcctggcccgcgaaggcgagccgccgcaaagcggcgtggtgctgctggccggcaccaaccaccacattcgtttattgaagaatggcacgctagcctatacggcagagccggtgaacgaaatttaccggccatcgatcgatgtgtttttcgaaagcgtggccagccactggaatggcgatgccgtcggtgtgctgctgaccggcatggggcgcgacggggcccagggcctcaaattgctacgtgaacaaggttatttgaccatcgcccaggatcagcaaagctcggcggtgtatggcatgcccaaagcggcggcggcgatcgatgctgctgttgaaattcgcccactggatagaattgcgccccggttgctggaggtctttgccaaatgaacatgacctctcgcggctggcttgcaggcagtaattcaggtgactgcacatgaatgatttacagatcgacgacatcaagaccgacgaaaacgccgccatggtgttgctggtcgacgaccaggccatgatcggtgaagccgtgcggcgtggcctggcccatgaagaaaatatcgacttccacttctgcgccgacccacaccaggcgattgcccaggcgatccgtatcaagccgaccgttatcctgcaggatctggtgatgccaggtctggacggcctgacactggtgcgcgagtaccgcaaccacccggccacgcagaacatcccgatcatcgtgctttccaccaaggaagacccgctgatcaagagcgcggcgttttcggccggggccaacgattatttggtcaagctgccggacaacatcgagctggtggcgcgcatccgctatcactcgcgctcctacatgaccctgttgcaacgggatgcggcttatcgcgcgttgcgggtcagccagcagcagctgttggacaccaacctggtgctgcaacggctgatgaactccgatggcctcacggggctgtccaaccgtcgccatttcgacgagtacctggaactggaatggcgccgtgccatgcgtgatcagactcagctgtcgttgttgatgattgatgtggatttcttcaagacctacaacgatagctttgggcatgtcgaaggtgacgaggctttgcgcaaggttgcggcgaccattcgtgaggccagcagtcggccttcggatttgccggcgcgctatgggggggaggagtttgccctggtgctgcctaatacctcgccgggcggggcgcggttggtggctgagaagctgcggatggcggttgccgcgctgaaaattccgcacattgcgccgactgaggggtcgagtctgaccatcagtattgggctgtcgaccatgacgccgcagcaggggacggattgtcggcaggtgatagtggcggcggataaggggttgtatacggctaagcataatgggcgcaatcaggtggggattgagtagggctgagtgcttgtgtacatatccgttgctgcggtcacggccactaatggttccgctcttacagcagggtcactttttgaaaagcgcaaaaagtaaccaaaaacgcttcgccccaacactcggcacctcgcctaggactcggtgtaccctcactcaggattcaatactgcgttcagccaacgtgtttgacggagcgccttagatcaaaagcaaaaacgcggcggccttaaaacccaccgaggttggggagcattactgggtaaaaatgtgggagggggcttgacctggcggtgtgtagtgatcgatctgtggctgtacactgcca

SEQ ID NO: 28 - protein, 547 aa, Pseudomonas fluorescens
MALRGITVKNWTLRQRILASFAVIIAIMLLMVVVSYSRLLKIETSQEAVRDDAVPGVYLSSMIRSAWVDSYLQTIDIIGLRDDKTFTNTDKNDYKSFEARIEQQMANYEKTIHGQADRMEFDNFKAAHINYNKVLAQVLERVEANDLPGANQLLEEQLTPIWTEGRMKLNDIITENKNVSDRATAAIDEAVLSAKISMAVSLLIAILAAGLCGLLLMRAIMAPMQRIVDILETMRDGDLSKRLNLERKDEFGAVETGFNDMMTELTALVSQAQRSSVQVTTSVTEIAATSKQQQATATETAATTTEIGATSREIAATSKDLVRTMTEVSTAADQASVAAGSGQQGLARMEETMHSVMGAADLVNAKLAILNEKAGNINQVVVTIVKVADQTNLLSLNAAIEAEKAGEYGRGFAVVATEVRRLADQTAVATYDIEQMVREIQSAVSAGVMGMDKFSEEVRRGMFEVQQVGEQLSQIIHQVQALAPRVLMVNEGMQAQATGAEQINHALVQLGDASSQTVESLRQASFAIDELSQVAVGLRSGVSRFKV

SEQ ID NO: 29 - protein, 170 aa, Pseudomonas fluorescens
MSELAAKRGAVPAAKKALFLVFHIGQERYALKATEVAEVLPRLPLKPIAHAPLWVAGIFAHRGALVPVIDLSALTFGNPAQARTSTRLVLVNYQPDAGSQARWLGLILEQATDTLRCDPAEFQPYGLANRQAPYLGPVREDALGLMQWIGVNDLLTDDVRAVLFSAELSV

SEQ ID NO: 30 - protein, 419 aa, Pseudomonas fluorescens
MSNDPRFFAFLKERIGLDVASVGEAIIERAVRQRSQIVQAPTPGEYWQHLQSSQDEQQALIEAVIVPETWFFRYPESFATLARLAKARLVDIKQMRALRILSLPCSTGEEPYSIAMALLDAGLAPHQFKVQGMDVSPLSVERARRGVYGKNSFRGGDIAFRDRHFTEYGDGFHIADRVREQVRLQVGNLLDPALLVNEAAYDFVFCRNLLIYFDQPTQKQVFDVLKGLTHVDGVLFIGPAEGSLLGRHGMRSIGVPQSFAFSRQGSPQLPEPAFIPTPAPTPPRSTAPISAKPRPFSTVSAHVLPIKATPSDAGTLLSRIATLANEGKSAEARAACEDYLNSHPPAAQVFYWLGLLSDVAGSALEAQGYYRKALYLEPQHPQALMHLAALLESRGDSAGARRLQARAARSERADSESKP

SEQ ID NO: 31 - protein, 232 aa, Pseudomonas fluorescens
MSNTEALDTTGLDLTLADTQAIDDCWNRIGIHGDKSCPLLADHIHCRNCSVYSAAATRLLDRYALQQDDRRPQAAEVDTEVVTRSLLMFRLGEEWLGIATRCLVEVAPLQPIHSLPHQRSRALLGVANVRGALVACLSLVELLGLDATSSGATGGRIMPRMLIIAAQDGPVVVPVDEVDGIHAIDERTLKAASASGTQASARFTQGVLPWKGRSLRWLDEAQLLSAVTRSLS

SEQ ID NO: 32 - protein, 755 aa, Pseudomonas fluorescens
MTPDQMRDASLLELFSLEADAQTQVLSAGLLALERNPTQADQLEACMRAAHSLKGAARIVGVDAGVSVSHVMEDCLVSAQENRLYLQPEHIDALLQGTDLLMRIATPGNDVGPADVEAYVALMERLLDPSQAPVNVAPSPEPAPVVEELPPEPEPAPPVNSEPPRQGKRMTEGGERVLRVTAERLNSLLDLSSKSLVETQRLKPYLASLQRLKRIQSQGIRALDTLDGQLKTQVLSLEAQEALADTRRLLSEAQALLAEKHAELDEFGWQAGQRAQVLYDTALACRMRPFADVLAGQVRMVRDLGRSLGKQVRLEIEGEKTQVDRDVLEKLEAPLTHLLRNAVDHGIEMPEQRLLAGKPAEGLIRLRASHQAGLLVLELSDDGNGVDLERLRGTIVDRHLSPVETALRLSEEELLTFLFLPGFSLRDTVTEVSGRGVGLDAVQHMVRQLRGAVVLEQTAGQGSRFHLEVPLTLSVVRSLVVEVGEEAYAFPLAHIERMCDLAPDDIVQLEGRQHFWHEGRHVGLVAASQLLQRPAGQSPSETLKVVVIRERDTVYGIAVERFIGERTLVVLPLDDRLGKVQDISAGALLDDGSVVLIVDVEDMLRSVDKLLNTGRLERIARRSQQTTEAPRKRVLVVDDSLTVRELQRKLLLNRGYEVAVAVDGMDGWNALRSEDFDLLITDIDMPRMDGIELVTLLRRDSRLQSLPVMVVSYKDREEDRRRGLDAGADYYLAKASFHDDALLDAVVELIGGARA

SEQ ID NO: 33 - protein, 336 aa, Pseudomonas fluorescens
MRIAIVNDMPLAVEALRRALSFEPAHQVVWVASNGLEAVQRCAELTPDLILMDLIMPVMDGVEATRQIMAETPCAIVIVTVDRQANVSRVFEAMGHGALDVVDTPPLGVGNPKDAAAPLLRKILNIGWLIGQRGTRVRAETLPARASGKRQSLVAIGSSAGGPAALEILLKGLPRDFPAAIVLVQHVDQVFAAGMAEWLSSASGLPVRLAREGEPPQSGVVLLAGTNHHIRLLKNGTLAYTAEPVNEIYRPSIDVFFESVASHWNGDAVGVLLTGMGRDGAQGLKLLREQGYLTIAQDQQSSAVYGMPKAAAAIDAAVEIRPLDRIAPRLLEVFAK

SEQ ID NO: 34 - protein, 333 aa, Pseudomonas fluorescens; identical to SEQ ID NO: 12

(19) United States
(12) Patent Application Publication
(10) Pub. No.: US 2004/0054165 A1
(43) Pub. Date: Mar. 18, 2004

(54) Bacterial polysaccharide and biofilm development

(76) Inventors: Paul Barton Rainey, Oxford (GB); Andrew Julien Spiers, Oxford (GB); Eleni Bantinaki, Crete (GR)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/332,288

(22) PCT Filed: Jul. 9, 2001
(86) PCT No.: PCT/GB01/03077

(30) Foreign Application Priority Data
Jul. 7, 2000 (GB) 0016842.7

(51) Int. Cl. C12Q 1/04; C12N 1/20; C08B 37/00

(52) U.S. Cl. 536/123.12; 435/34; 435/252.1

(57) ABSTRACT

The invention is concerned with the identification of a novel class of bacterial polysaccharide biosynthetic operons and an ovel clas of regulatory operons involved with polysaccharide biosynthesis, bacterial attachment and biofilm development. Bacterial strains which possess a polysaccharide biosynthetic operon of the type provide by the invention are capable of producing polysaccharide wtih industrial implications. Bacterial strains which possess a regulatory operon of the type provided by the invention may be targeted by pharmaceutical/chemical agents to prevent bacterial attachment and biofilm development.

### FIELD OF THE INVENTION

The present invention is concerned with the identification of a novel class of bacterial polysaccharide biosynthetic operons and a novel class of regulatory operons involved with polysaccharide biosynthesis, bacterial attachment and biofilm development. Bacterial strains which possess a polysaccharide biosynthetic operon of the type provided by the invention are capable of producing a polysaccharide with industrial implications. Bacterial strains which possess a regulatory operon of the type provided by the invention may be targeted by pharmaceutical/chemical agents to prevent bacterial attachment and biofilm development.

Also provided by the invention is a process for the production of the polysaccharide, methods of isolating/engineering polysaccharide-producing bacterial strains and also novel enzymes involved in polysaccharide biosynthesis, methods to screen chemical libraries for pharmaceutical/chemical agents to prevent bacterial attachment and biofilm development and also novel proteins involved in the regulation of polysaccharide synthesis, bacterial attachment and biofilm development.

### BACKGROUND TO THE INVENTION

Cellulose and modified celluloses are valuable polymers with a wide range of applications, for example, in the food, clothing, paint and paper-producing industries and also in medicine, particularly the production of artificial veins and wound dressings. Most cellulose is derived from plants, but there is increasing interest in bacterial-derived celluloses, because of their value in the production of speciality items, such as certain papers, natural thickening agents for the food industry and artificial tissues for use in medicine. The vast majority of industrial uses are, in fact, for modified celluloses. It is the substitution pattern on the primary cellulosic backbone that creates the various properties exhibited by industrially useful modified celluloses.

Despite the obvious usefulness of bacterial cellulose, to date only a single cellulose-producing bacterium, Acetobacter, is exploited by industry (see general review by Ross, P. et al. Microbiol. Rev. 1991, 55: 35-50). It is therefore an object of the present invention to provide an alternative source of industrially useful cellulose-like bacterial polysaccharides.

The present inventors have identified a novel class of polysaccharide biosynthetic operon, the genetic organisation of which differs significantly from the cellulose biosynthetic operon of Acetobacter. Of key importance, the inventors have identified a second locus involved in the regulation of polysaccharide biosynthesis. Bacterial strains which possess the polysaccharide biosynthetic operon and in which enzyme-encoding genes of the operon are expressed, including certain strains of Pseudomonas and E. coli, are capable of producing large amounts of a particular type of polysaccharide.

The inventors have also determined that the second regulatory locus is involved in bacterial attachment. Bacterial attachment is a necessary first step in the development of biofilms. Isogenic pairs of bacterial strains, one of which lacks the regulatory locus, can provide a screen for chemical or pharmaceutical agents that block attachment and biofilm growth.

### DESCRIPTION OF THE INVENTION

Glucan-Like Polysaccharide

In a first aspect the invention provides a glucan-like polysaccharide produced by an exopolysaccharide-producing bacterial strain, said bacterial strain being characterised in that it expresses one or more enzyme-encoding genes of a wss-like operon.

The polysaccharide of the invention can be derived from any bacterial strain which expresses one or more enzyme-encoding genes of a wss-like operon and is usually produced as an extracellular polysaccharide (or exopolysaccharide). Bacterial strains which produce the polysaccharide of the invention may be referred to herein as “exopolysaccharide-producing bacterial strains”.

The term “exopolysaccharide-producing bacterial strain” encompasses any bacterial strain which has a wss-like polysaccharide biosynthetic operon and in which one or more of the genes encoding subunits of cellulose synthase are expressed. Also encompassed within the scope of the term “exopolysaccharide-producing bacterial strain” are recombinant strains which have been engineered to express one or more enzyme-encoding genes of a wss-like operon and also strains which have a wss-like operon and have been engineered to over-express a regulator of the wss-like operon. The characteristics and construction of these recombinant strains will be more fully explained below. Finally, the term “exopolysaccharide-producing bacterial strain” also encompasses mutagenized strains derived from parent strains having a wss-like operon, for example strains wherein one or more genes of the wss-like operon which are not expressed in the parent strain are expressed in the mutagenized strain. The construction of such mutagenized strains will be described in more detail below.

Different exopolysaccharide producing bacterial strains may produce glucan-like polysaccharides which are slightly different in terms of structure and/or chemical composition. It is to be understood that the term “glucan-like polysaccharide” does not refer to any one single substance but is rather a generic term which encompasses exopolysaccharides produced by a wide range of exopolysaccharide producing bacterial strains.

In a preferred embodiment the exopolysaccharide-producing bacterial strain is an “evolved variant” of an ancestral strain, wherein the ancestral strain has a wss-like polysaccharide biosynthetic operon but does not naturally produce the glucan-like polysaccharide provided by the invention. As exemplified herein, the inventors have observed that a wild-type strain which possesses a wss-like operon can evolve into polysaccharide producing evolved variants when cultured in a novel environment, such as the static broth culture described herein. Exopolysaccharide-producing evolved variants of Pseudomonas sp. may be distinguished from the corresponding ancestral strain, for example by virtue of a characteristic “wrinkly spreader” (WS) morphology (described by Rainey & Travisano, 1998, Nature, 394: 69-72). Procedures for the isolation of exopolysaccharide-producing evolved variant strains will be described hereinbelow.

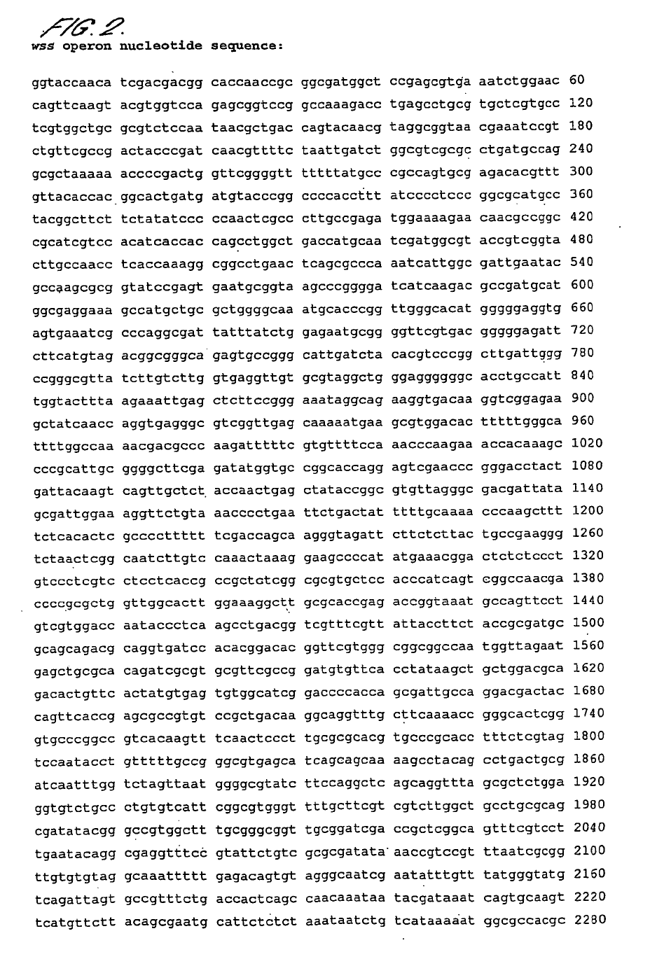

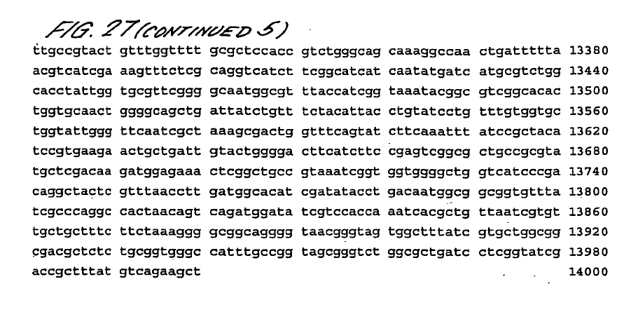

“Wss-like operon” is a generic term used herein to describe a novel class of bacterial operons containing genes which encode enzymes involved in the biosynthesis of glucan-like polysaccharides. In general terms, wss-like operons are distinguished from the cellulose biosynthetic operons of Acetobacter because they lack a gene encoding the enzyme responsible for cellulose crystallization. Encompassed within the term “wss-like operon” are operons which are homologous to the wss operon of Pseudomonas fluorescens, the complete nucleotide sequence of which is given herein. Preferably, the wss-like operon comprises a sequence of nucleotides which shares at least 50% nucleotide sequence identity with the sequence of nucleotides shown in FIG. 2 (SEQ ID NO: 1) or FIG. 27 (SEQ ID NO: 26). Even more preferably, the wss-like operon may comprise a sequence of nucleotides at least 55%, at least 60%, at least 65%, at least 70%, at least 75%, at least 80%, at least 90% or at least 95% identical to the P. fluorescens wss operon shown in FIG. 2 (SEQ ID NO: 1) or the E. coli yhj operon shown in FIG. 27 (SEQ ID NO: 26).

In accordance with the invention, percent identity of nucleotide and amino acid sequences may be calculated based on an optimal alignment of the sequences to be compared, taking account of nucleotide/amino acid insertions and deletions. An optimal alignment can be assembled using the BLAST algorithm which is well known in the art.

Percentage nucleotide identity may be calculated by comparing entire wss or yhj operon sequences or by comparing the coding regions of the individual genes of the operons. In the latter case, the coding regions of homologous genes should be compared. A value for the overall percentage sequence identity may then be derived by taking an average over all the individual homologous coding regions. A complete annotation of the P. fluorescens wss operon, showing the positions of the coding regions is listed elsewhere in this specification. Similarly, the positions of the individual coding regions within the E. coli yhj operon are given below.

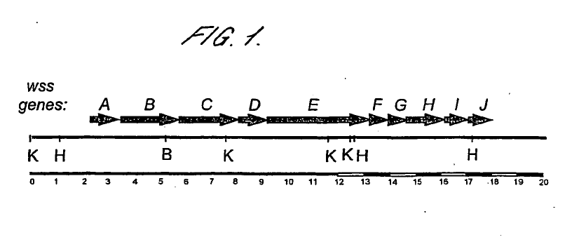

As well as having a significant degree of nucleotide sequence identity with the nucleotide sequences illustrated in FIG. 2 (SEQ ID NO: 1) or FIG. 27 (SEQ ID NO: 26), the wss-like operon may or may not share substantial organisational similarity with the Pseudomonas fluorescens wss and E. coli yhj operons, meaning that the homologous genes are arranged in the same order within the operon. “Substantial organisational similarity” with the P. fluorescens wss operon should be taken to mean that at least the genes encoding the subunits of cellulose synthase should be arranged in the same order as they are in the Pseudomonas fluorescens wss operon. As illustrated in FIG. 1, the genes encoding enzyme subunits which are homologous to cellulose synthase subunits from other bacterial species are arranged 5′-wssB-wssC-wssE-3′. These genes and the enzymes they encode may be designated herein as “cellulose synthases” but this is on the basis of homology to cellulose synthase genes from other bacterial species. The use of this nomenclature should not be taken to imply that the final polysaccharide products synthesised by the action of these enzymes are pure celluloses (i.e. pure 1-4 β-linked glucan) or that the activity of these enzymes is limited to the synthesis of pure celluloses. Wss-like operons may be distinguished from known bacterial cellulose biosynthetic operons (e.g. the acetobacter Bcs operon) by the absence of the gene responsible for cellulose crystallization and the presence of additional genes (wss G H I; alg F I J) whose enzyme products are involved in acetylation of polysaccharides. Although it is possible that the enzymes encoded by the wssA-E genes may produce a product which is a substantially pure cellulose, the enzymes encoded by wss G H I might then modify this product.

The term “enzyme-encoding gene” as used herein refers to a gene which encodes a protein product which functions as an enzyme or as an enzyme subunit.

Knowledge of the primary nucleotide sequence and the structural organisation of the P. fluorescens wss and E. coli yhj operons enables the identification of other microorganisms which have a wss-like operon. This can be accomplished using a variety of techniques which are known in the art. In order to find out whether a given microorganism has a wss-like operon, a labelled nucleic acid probe corresponding to a part of the wss operon could be used to probe a Southern blot of genomic DNA. Genomic DNA fragments containing the wss-like operon, or parts thereof, could then be isolated by probing a library of genomic DNA from the organism, e.g. a library of bacterial chromosomal DNA fragments. Preferably, the labelled probe fragment would correspond to a region of one of the enzyme-encoding genes of the wss operon which is highly conserved cross-species. Procedures for the preparation of suitable labelled probe fragments, Southern blotting, construction of chromosomal libraries, cross-species library screening, recovery of positive clones and sequencing of the DNA inserts would be well known to one skilled in the art (see, for example, Sambrook, Fritsch & Maniatis, Molecular Cloning: A Laboratory Manual, Second Edition (1989) Cold Spring Harbor Laboratory Press, Cold Spring Harbor, N.Y.; F. M. Ausubel et al. (eds.), Current Protocols in Molecular Biology, John Wiley & Sons, Inc. (1994)).

As an alternative to the library screening approach, oligonucleotide primers corresponding to suitable regions of the P. fluorescens wss operon or the E. coli yhj operon could be used for PCR amplification of homologous wss operon sequences from DNA isolated from other bacterial species. Again, standard procedures for purification of chromosomal DNA, PCR amplification and cloning and sequencing of PCR products are well known in the art (Sambrook, et al. supra; Ausubel, et al. supra).

Finally, a vast amount of publicly available nucleotide sequence data from a wide range of different organisms is accessible electronically via the Internet, see for example, www.ncbi.nlm.nih.gov. Therefore, one approach to identifying wss-like operons in other species would be a “bioinformatics” approach using the nucleotide sequence of the P. fluorescens wss operon or the E. coli yhj operon or a fragment thereof to perform a search of the available databases.

As will be illustrated in the Examples included herein, the glucan-like polysaccharide of the invention is obtainable by,

(a) culturing an exopolysaccharide-producing bacterial strain;

(b) lysing the bacteria overnight at 37° C. in a lysis solution of 20 mM Tris.HCl pH8.0, 5 mM MgCl₂, 0.5% Sarkosyl, 1 mg/ml fresh lysozyme;

(c) treating the lysed sample obtained in step (b) with DNase and RNase; and

(d) incubating the sample obtained in step (c) for 24 hr with a second lysis solution of 500 mM EDTA pH 9.0, 1% Sarkosyl, 1.5 mg/ml Proteinase K.

However, it is to be understood that the invention is in no way limited to polysaccharide produced according to the process steps listed as steps (a) to (d) above.

The polysaccharide provided by the invention may also be identified on the basis of positive staining with a staining reagent which specifically stains polysaccharides having a predominantly β-linked glucan structure, for example calcofluor.

In terms of chemical structure, the polysaccharide of the invention is defined as a polymer having glucose residues as the main constituent of the polymeric backbone, wherein the glucose residues are linked by glycosidic bonds, which may be of either α or β configuration and may be of any of the following linkage varieties; 1-3,1-4 or 1-6. In addition, the polymer backbone may have integrated into its structure other hexose sugar residues or derivatised hexose residues, for example galactose, mannose or galacturonic acid. The residues in the backbone may further be substituted at any position by hexose or pentose residues, derivatised hexose or pentose residues or other functional groups including, but not limited to, acidic functional groups such as acetyl groups. The polymer may be of any degree of polymerisation.

The polysaccharide of the invention may be referred to herein as being a “glucan-like” polymer, since its structure is based predominantly, though not exclusively, on a β-linked glucan backbone that may be substituted with, for example, other sugars and also functional groups. It is to be understood that the polysaccharide of the invention is not a pure cellulose, i.e. unsubstituted, since it is generally observed to be amorphous in structure rather than crystalline.

The polysaccharide polymers provided by the invention may have a plurality of structures and associated properties. These properties may include, but not exclusively, degree of crystallinity, degree of polymerisation, degree, extent and pattern of substitution, ability to form fluids of varying viscosity when the polymers are integrated into a fluid substance, ability to control enzymic reaction rates when in an environment with active enzyme components, ability to control or otherwise alter the tensile strength of a substance when integrated or mixed with such a substance or product, ability to control or otherwise alter the motility of bacteria in an environment into which the polymer has been introduced, ability to control or otherwise alter the attachment of bacteria to surfaces in an environment into which the polymer has been introduced ability to control or otherwise alter an organisms ability to metabolise any substance when the polymer is in the environment of the substance.

Methods of Isolating Exopolysaccharide-Producing Bacterial Strains

In a still further aspect, the invention provides a method of isolating an exopolysaccharide-producing bacterial strain, which method comprises the steps of:

(a) growing a wild type bacterial strain, the genome of which comprises a wss-like operon, in a static broth culture;

(b) isolating a variant strain having wrinkly spreader morphology;

(c) optionally, screening colonies of the wrinkly spreader morph obtained in step (b) for the production of polysaccharide.

The above method of the invention can be used to isolate exopolysaccharide-producing variants of any wild type bacterial strain having a wss-like operon. This bacterial strain is commonly referred to herein as an “ancestral” strain. Advantageously, the method of the invention can be used to isolate exopolysaccharide-producing strains of Pseudomonas and E. coli. Preferred ancestral Pseudomonas strains which can be used in the method of the invention include Pseudomonas fluorescens SBW25. Preferred E. coli ancestral strains which can be used in the method of the invention include E.coli K12.

Ancestral P. fluorescens SBW25 shows no evidence of cellulose or modified cellulose production in vitro (although available data suggests that the wss operon is active when the bacterium is associated with plant surfaces). Mutant forms which were later shown to produce a glucan-like polysaccharide were obtained following selection of the ancestral (smooth; SM) genotype in stationary broth vials over the course of 10 days (see Rainey & Travisano, Nature, 394, 69-72). Among a diverse collection of mutant genotypes that evolve are a class known as wrinkly spreaders (WS). The name of this class of mutants reflects their morphology on agar plates and in static broth microcosms they colonise the interface between liquid and air. Subsequent genetic analysis of WS morphology revealed the wss operon, the wsp operon and various other genes described in this application with a role in the production of the glucan-like polysaccharide.

The distribution of the wss operon among other Pseudomonas strains has been examined using gene probes made from the P. fluorescens SBW25 wss operon. Positive signals can be observed in a number of strains. The sequence of the wss genes can therefore be used to identify other glucan-like polysaccharide-producing bacteria. The inventors have also used the evolutionary selection procedure to show that strains with wss genes are capable of generating the WS phenotype. Thus evolutionary selection experiments also have the potential to reveal a latent ability to produce a glucan-like polysaccharide in a diverse range of Pseudomonas strains.

The invention further provides an exopolysaccharide-producing bacterial strain which is obtainable by the method of the invention and the glucan-like polysaccharide produced by such a bacterial strain.

In a still further aspect, the invention also provides a method of isolating an exopolysaccharide-producing bacterial strain which comprises the steps of exposing a bacterial strain, the genome of which comprises a wss-like operon, to a chemical mutagen; and identifying a mutant which produces a polysaccharide according to the invention.

The chemical mutagen can be any mutagenic agent known in the art, preferably an agent which is known for use in random mutagenesis of bacterial chromosomes or a mixture of such agents.

In one embodiment, the step of identifying a mutant which produces polysaccharide can be accomplished by plating out a large number of mutant colonies and staining with chemical stain specific for the polysaccharide (e.g. calcofluor, Congo red). Alternatively, exopolysaccharide-producing mutants can be identified by looking for variants having the wrinkly spreader colony morphology. This is a preferred method of identifying mutant strains of Pseudomonas. Following exposure to a chemical mutagen, the mutagenized bacteria may optionally be grown under culture conditions which favour the growth of exopolysaccharide-producing variants, for example the static culture conditions described herein (see Example 1).

Also provided by the invention are exopolysaccharide-producing evolved variant and mutant strains which are obtainable according to the above-described methods, and the glucan-like polysaccharide produced by such strains.

Novel Genes and Proteins

The inventors have further identified a number of novel genes which encode components of the polysaccharide biosynthetic pathway of P. fluorescens.

Therefore, according to a further aspect of the invention there is provided an isolated nucleic acid molecule comprising the sequence of nucleotides from position 2200 to 18000 of the nucleotide sequence illustrated in FIG. 2 (SEQ ID NO: 1).

FIG. 2 (SEQ ID NO: 1) shows the nucleotide sequence for a contiguous piece of DNA of 20,306 bp from the genome of Pseudomonas fluorescens SBW25. The wss operon, encoding genes homologous to known cellulose biosynthetic genes from other bacterial species and also associated genes, is located approximately between 2,200-18,000 bp. The operon consists of ten genes, designated wssA-J. A schematic figure showing the arrangement of the operon is shown in the accompanying FIG. 1.

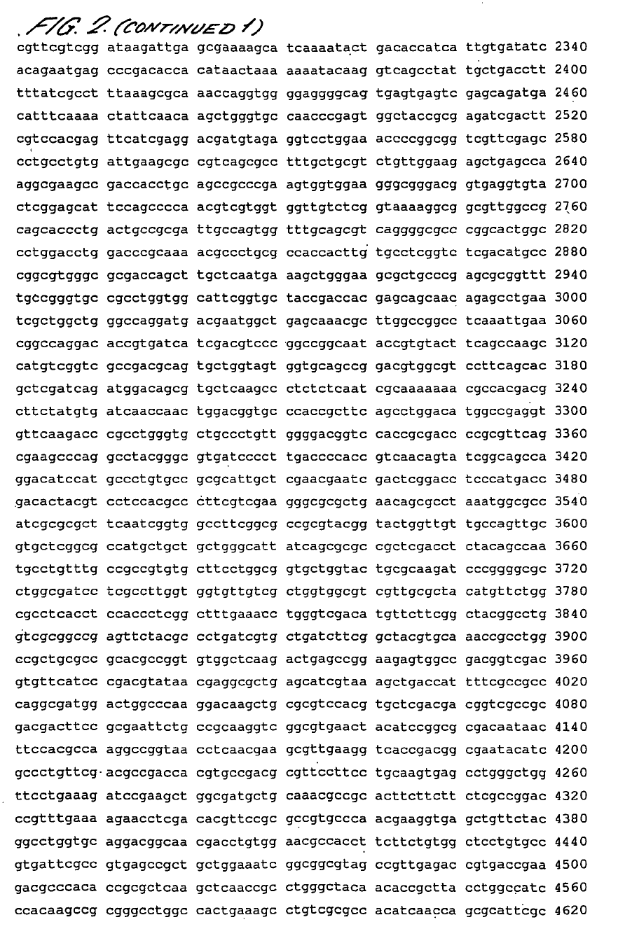

Isolated individual genes from the wss operon are also encompassed within the scope of the invention. Thus, the invention provides an isolated nucleic acid molecule encoding a WssA protein, said protein comprising the sequence of amino acids from position 145 to position 344 of the amino acid sequence illustrated in FIG. 3 (SEQ ID NO: 2) or from position 2 to position 344 of the amino acid sequence illustrated in FIG. 3 (SEQ ID NO: 2). Preferably the nucleic acid molecule comprises the sequence of nucleotides from position 2876 to 3478 or from position 2444 to 3478 of the nucleic acid sequence illustrated in FIG. 2 (SEQ ID NO: 1).

Also provided by this aspect of the invention is a WssA protein comprising the sequence of amino acids from position 2 to 344 of the amino acid sequence illustrated in FIG. 3 (SEQ ID NO: 2) or from position 145 to position 344 of the amino acid sequence illustrated in FIG. 3 (SEQ ID NO: 2). The invention further provides a WssA protein encoded by a nucleic acid molecule according to the invention.

The amino acid sequence shown in FIG. 3 (SEQ ID NO: 2) is the predicted translation of the longest open reading frame of the Pseudomonas fluorescens wssA gene. Translation of the WssA protein in vivo is expected to initiate at the first possible in-frame methionine codon (amino acid residue number 145 in the wssA predicted translated). However, it is possible that translation may initiate at the first in-frame valine codon (amino acid residue number 1 in the wssA translated sequence). In this case, the initiating amino acid of the protein product would still be methionine, not valine. Hence, the invention also provides a WssA protein comprising the amino acid sequence from position 2 to position 344 of the amino acid sequence illustrated in FIG. 3 (SEQ ID NO: 2) but having an additional N-terminal methionine residue.

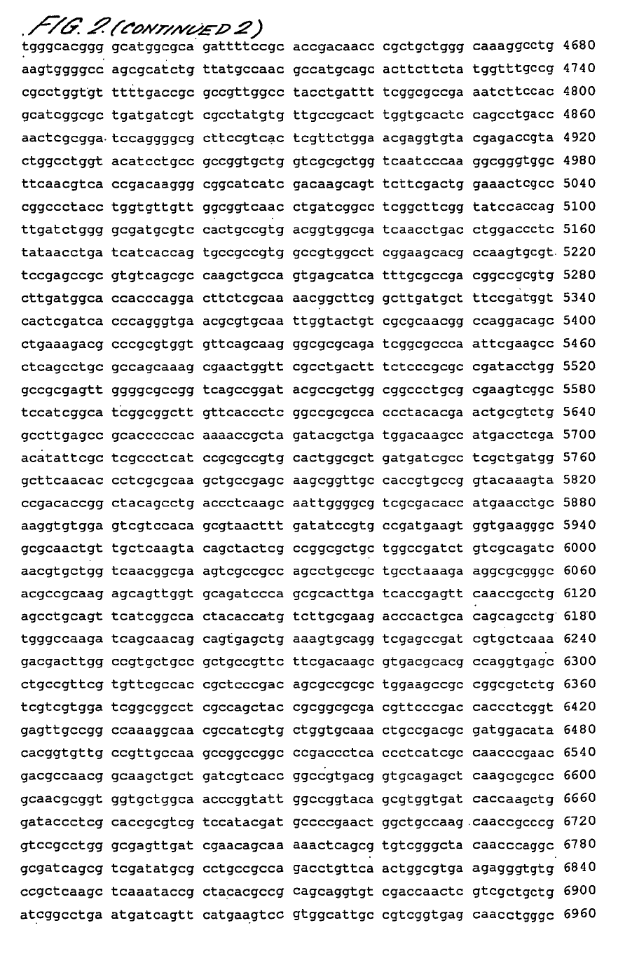

The invention further provides an isolated nucleic acid molecule encoding a WssB protein (sharing homology with cellulose synthase subunit A), said protein comprising the sequence of amino acids illustrated in FIG. 4 (SEQ ID NO: 3). Preferably the nucleic acid molecule comprises the sequence of nucleotides from position 3475 to 5694 of the nucleic acid sequence illustrated in FIG. 2 (SEQ ID NO: 1).

Also provided by this aspect of the invention is a WssB protein comprising the sequence of amino acids illustrated in FIG. 4 (SEQ ID NO: 3). The invention further provides a WssB protein encoded by a nucleic acid molecule according to the invention.

The amino acid sequence shown in FIG. 4 (SEQ ID NO: 3) is the predicted translation of part of the longest open reading frame of the Pseudomonas fluorescens wssB gene. Translation of the WssB protein in vivo is expected to initiate at the first possible in-frame methionine codon.

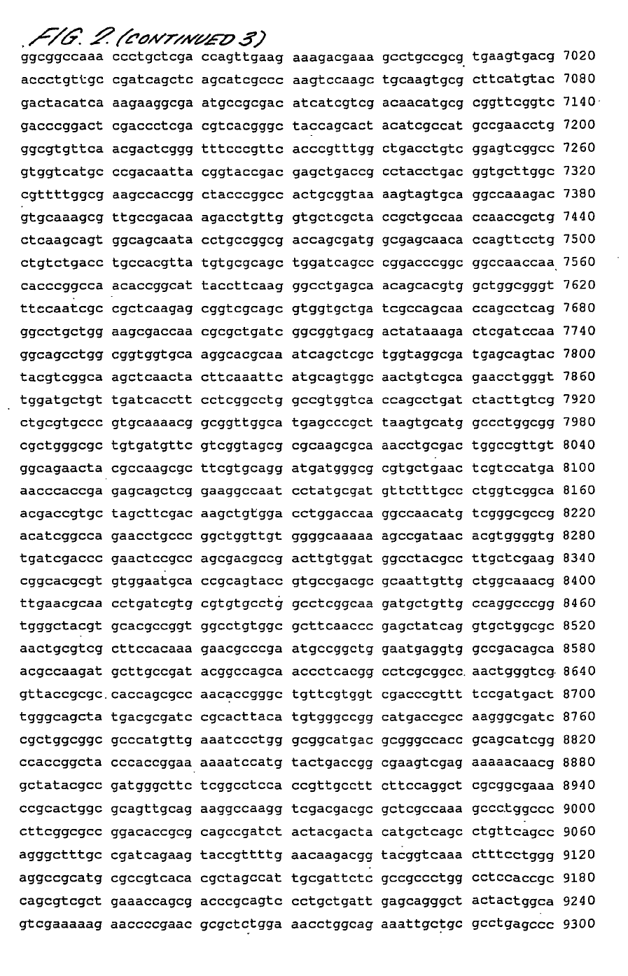

The invention further provides an isolated nucleic acid molecule encoding a WssC protein (sharing homology with cellulose synthase subunit B), said protein comprising the sequence of amino acids from position 89 to position 689 of the amino acid sequence illustrated in FIG. 5 (SEQ ID NO: 4) or from position 2 to position 689 of the amino acid sequence illustrated in FIG. 5 (SEQ ID NO: 4). Preferably the nucleic acid molecule comprises the sequence of nucleotides from position 5884 to 7953 or from position 6148 to 7953 of the nucleic acid sequence illustrated in FIG. 2 (SEQ ID NO: 1).

Also provided by this aspect of the invention is a WssC protein comprising the sequence of amino acids from position 89 to position 689 of the amino acid sequence illustrated in FIG. 5 (SEQ ID NO: 4) or from position 2 to position 689 of the amino acid sequence illustrated in FIG. 5 (SEQ ID NO: 4). The invention further provides a WssC protein encoded by a nucleic acid molecule according to the invention.

The amino acid sequence shown in FIG. 5 (SEQ ID NO: 4) is the predicted translation of part of the longest open reading frame of the Pseudomonas fluorescens wssC gene. Translation of the WssC protein in vivo is expected to initiate at the first possible in-frame methionine codon (amino acid residue number 89 in the wssC translated sequence). However, it is possible that translation may initiate at the first in-frame valine codon (amino acid residue number 1 in the wssC translated sequence). In this case, the initiating amino acid of the protein product would still be methionine, not valine. Hence, the invention also provides a WssC protein comprising the amino acid sequence from position 2 to position 692 of the amino acid sequence illustrated in FIG. 5 (SEQ ID NO: 4) but having an additional N-terminal methionine residue.

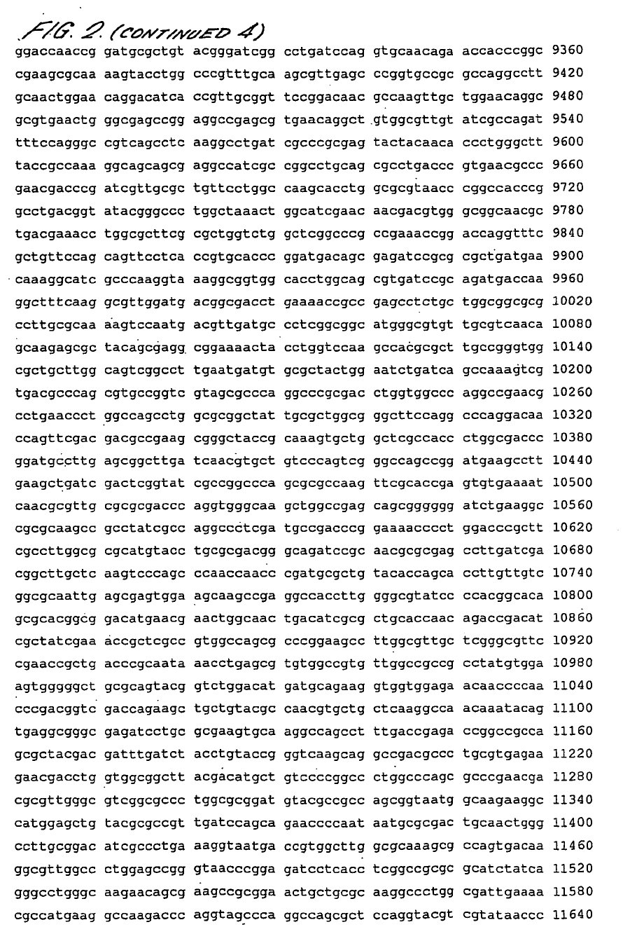

According to a further aspect of the invention there is provided an isolated nucleic acid molecule encoding a WssD protein (sharing homology with D-family cellulase associated with cellulose synthases), said protein comprising the sequence of amino acids from position 39 to position 436 of the amino acid sequence illustrated in FIG. 6 (SEQ ID NO: 5) or from position 2 to position 436 of the amino acid sequence illustrated in FIG. 6 (SEQ ID NO: 5). Preferably the nucleic acid molecule comprises the sequence of nucleotides from position 7884 to 9146 or from position 7950 to 9146 of the nucleic acid sequence illustrated in FIG. 2 (SEQ ID NO: 1).

Also provided by this aspect of the invention is a WssD protein comprising the sequence of amino acids from position 39 to position 436 of the amino acid sequence illustrated in FIG. 6 (SEQ ID NO: 5) or from position 2 to position 436 of the amino acid sequence illustrated in FIG. 6 (SEQ ID NO: 5). The invention further provides a WssD protein encoded by a nucleic acid molecule according to the invention.

The amino acid sequence shown in FIG. 6 (SEQ ID NO: 5) is the predicted translation of part of the longest open reading frame of the Pseudomonas fluorescens wssD gene. Translation of the WssD protein in vivo is expected to initiate at the first possible in-frame methionine codon (amino acid residue number 39 in the wssD translated sequence). However, it is possible that translation may initiate at the first in-frame valine codon (amino acid residue number 1 in the wssD translated sequence). In this case, the initiating amino acid of the protein product would still be methionine, not valine. Hence, the invention also provides a WssD protein comprising the amino acid sequence from position 2 to position 436 of the amino acid sequence illustrated in FIG. 6 (SEQ ID NO: 5) but having an additional N-terminal methionine residue.

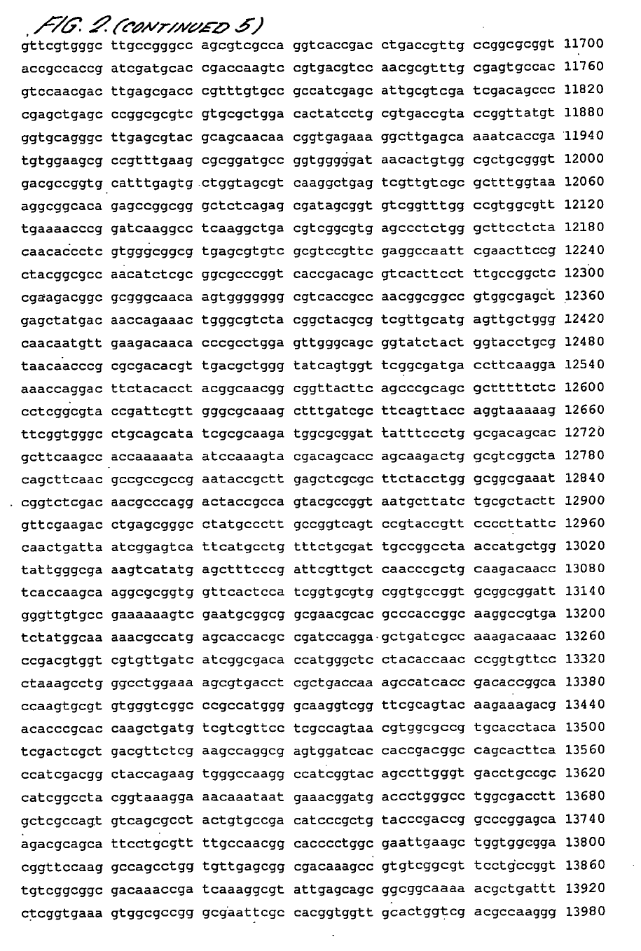

According to a further aspect of the invention there is provided an isolated nucleic acid molecule encoding a WssE protein (sharing homology with cellulose synthase subunit C), said protein comprising the sequence of amino acids illustrated in FIG. 7 (SEQ ID NO: 6). Preferably the nucleic acid molecule comprises the sequence of nucleotides from position 9128 to 12967 of the nucleic acid sequence illustrated in FIG. 2 (SEQ ID NO: 1).

Also provided by this aspect of the invention is a WssE protein comprising the sequence of amino acids illustrated in FIG. 7 (SEQ ID NO; 6). The invention further provides a WssE protein encoded by a nucleic acid molecule according to the invention.

The amino acid sequence shown in FIG. 7 (SEQ ID NO: 6) is a predicted translation of part of the longest open reading frame of the Pseudomonas fluorescens wssE gene. Translation of the WssE protein in vivo is expected to initiate at the first possible in-frame methionine codon (amino acid residue number 1 in the wssE translated sequence).

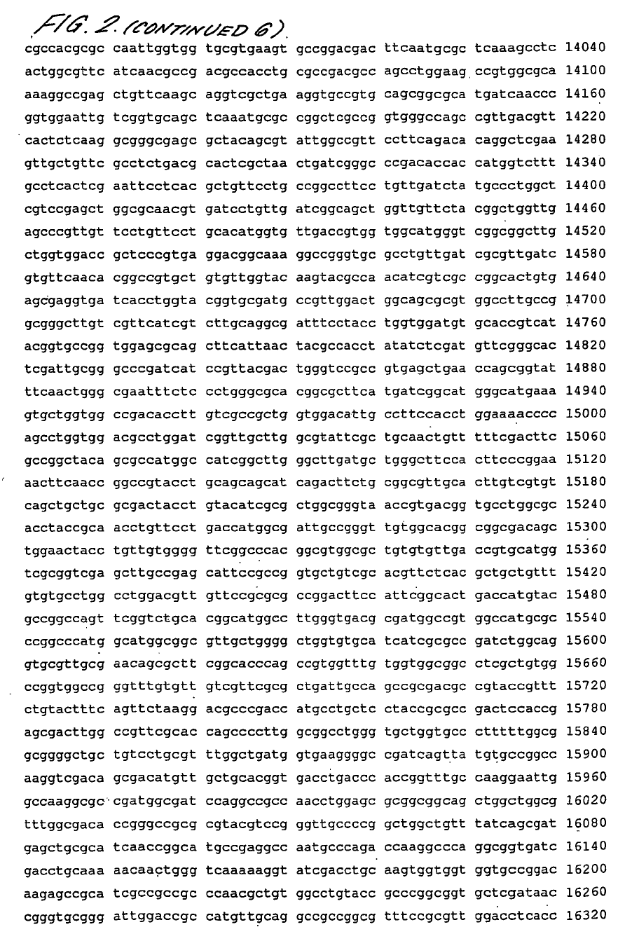

The invention still further provides an isolated nucleic acid molecule encoding a WssF protein, said protein comprising the sequence of amino acids illustrated in FIG. 8 (SEQ ID NO: 7). Preferably the nucleic acid molecule comprises the sequence of nucleotides from position 12984 to 13649 of the nucleic acid sequence illustrated in FIG. 2 (SEQ ID NO: 1).

Also provided by this aspect of the invention is an isolated WssF protein comprising the sequence of amino acids illustrated in FIG. 8 (SEQ ID NO: 7). The invention further provides a WssF protein encoded by a nucleic acid molecule according to the invention.

The amino acid sequence shown in FIG. 8 (SEQ ID NO: 7) is a predicted translation of part of the longest open reading frame of the Pseudomonas fluorescens wssF gene. Translation of the WssF protein in vivo is expected to initiate at the first possible in-frame methionine codon (amino acid residue number 1 in the wssF translated sequence).

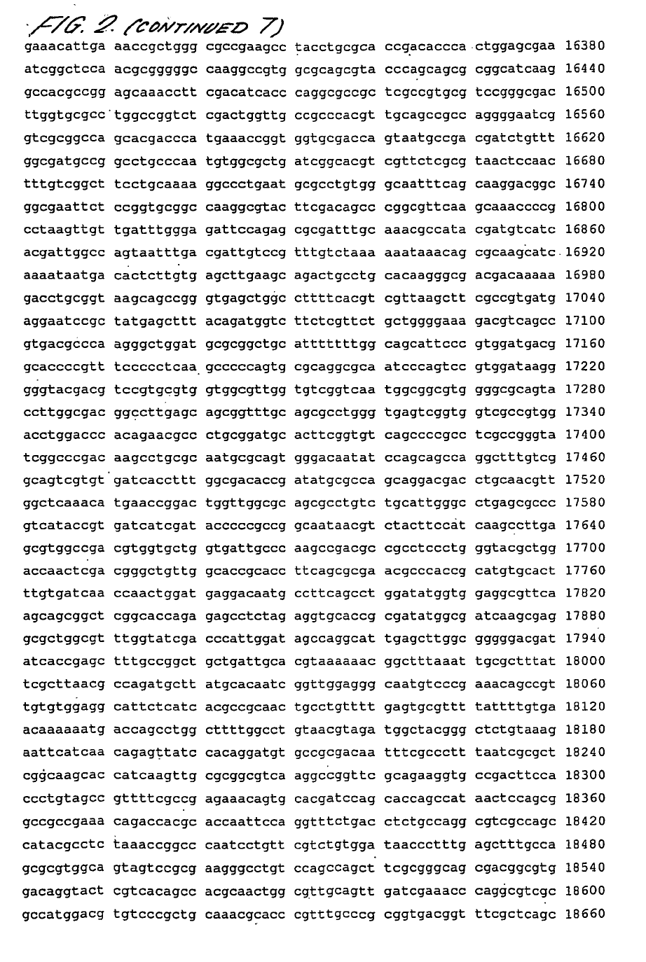

The invention further provides an isolated nucleic acid molecule encoding a WssG protein, said protein comprising the sequence of amino acids illustrated in FIG. 9 (SEQ ID NO: 8). Preferably the nucleic acid molecule comprises the sequence of nucleotides from position 13649 to 14314 of the nucleic acid sequence illustrated in FIG. 2 (SEQ ID NO: 1).

Also provided by this aspect of the invention is an isolated WssG protein comprising the sequence of amino acids illustrated in FIG. 9 (SEQ ID NO: 8). The invention further provides a WssG protein encoded by a nucleic acid molecule according to the invention.

The amino acid sequence shown in FIG. 9 (SEQ ID NO: 8) is a predicted translation of part of the longest open reading frame of the Pseudomonas fluorescens wssG gene. Translation of the WssG protein in vivo is expected to initiate at the first possible in-frame methionine codon (amino acid residue number 1 in the wssG translated sequence).

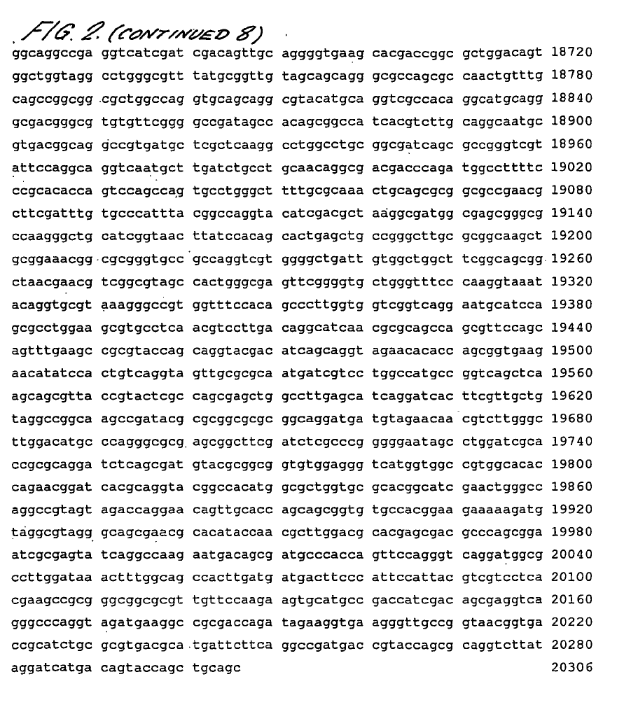

The invention further provides an isolated nucleic acid molecule encoding a WssH protein, said protein comprising the sequence of amino acids illustrated in FIG. 10 (SEQ ID NO: 9). Preferably the nucleic acid molecule comprises the sequence of nucleotides from position 14332 to 15738 of the nucleic acid sequence illustrated in FIG. 2 (SEQ ID NO: 1).

Also provided by this aspect of the invention is an isolated WssH protein comprising the sequence of amino acids illustrated in FIG. 10 (SEQ ID NO: 9). The invention further provides a WssH protein encoded by a nucleic acid molecule according to the invention.

The amino acid sequence shown in FIG. 10 (SEQ ID NO: 9) is a predicted translation of part of the longest open reading frame of the Pseudomonas fluorescens wssH gene. Translation of the WssH protein in vivo is expected to initiate at the first possible in-frame methionine codon (amino acid residue number 1 in the wssH translated sequence).

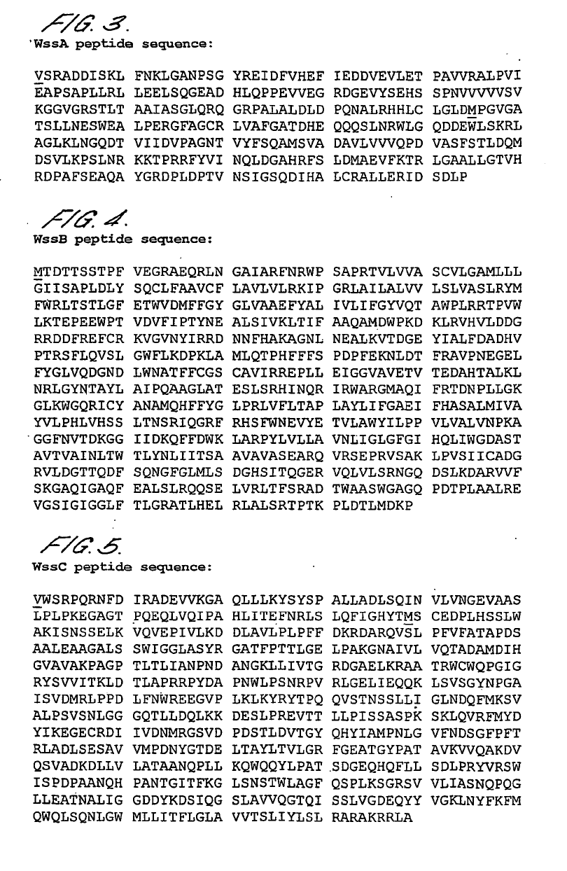

The invention further provides an isolated nucleic acid molecule encoding a WssI protein, said protein comprising the sequence of amino acids illustrated in FIG. 11 (SEQ ID NO: 10). Preferably the nucleic acid molecule comprises the sequence of nucleotides from position 15751 to 16875 of the nucleic acid sequence illustrated in FIG. 2 (SEQ ID NO: 1).

Also provided by this aspect of the invention is an isolated WssI protein comprising the sequence of amino acids illustrated in FIG. 11 (SEQ ID NO: 10). The invention further provides a WssI protein encoded by a nucleic acid molecule according to the invention.

The amino acid sequence shown in FIG. 11 (SEQ ID NO: 10) is a predicted translation of part of the longest open reading frame of the Pseudomonas fluorescens wssI gene. Translation of the WssI protein in vivo is expected to initiate at the first possible in-frame methionine codon (amino acid residue number 1 in the wssI translated sequence).

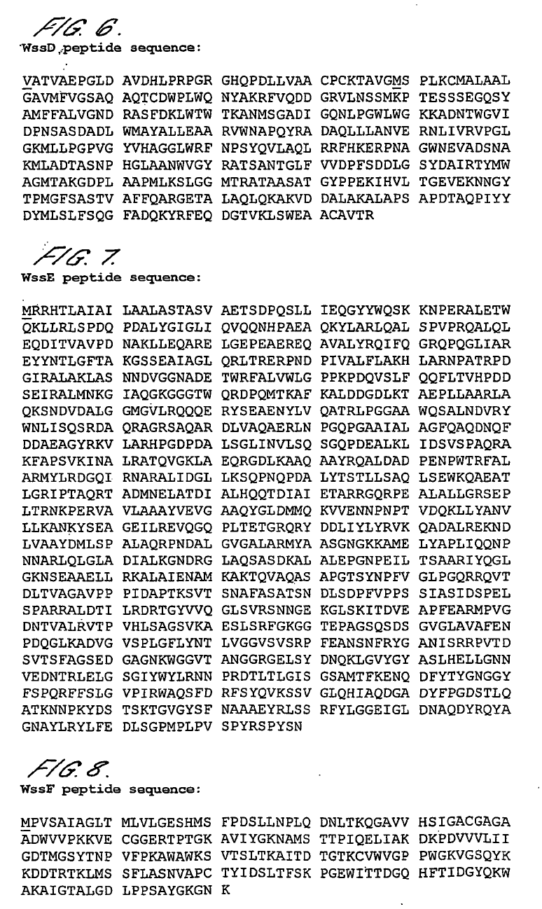

The invention further provides an isolated nucleic acid molecule encoding a WssJ protein, said protein comprising the sequence of amino acids from position 39 to position 324 of the amino acid sequence illustrated in FIG. 12 (SEQ ID NO: 11) or from position 2 to position 324 of the amino acid sequence illustrated in FIG. 12 (SEQ ID NO: 11). Preferably the nucleic acid molecule comprises the sequence of nucleotides from position 16938 to 17912 or from position 17052 to 17912 of the nucleic acid sequence illustrated in FIG. 2 (SEQ ID NO: 1).

Also provided by this aspect of the invention is a WssJ protein comprising the sequence of amino acids from position 39 to position 324 of the amino acid sequence illustrated in FIG. 12 (SEQ ID NO: 11) or from position 2 to position 324 of the amino acid sequence illustrated in FIG. 12 (SEQ ID NO: 11). The invention further provides a WssJ protein encoded by a nucleic acid molecule according to the invention.

The amino acid sequence shown in FIG. 12 (SEQ ID NO: 11) is the predicted translation of part of the longest open reading frame of the Pseudomonas fluorescens wssJ gene. Translation of the WssJ protein in vivo is expected to initiate at the first possible in-frame methionine codon (amino acid residue number 39 in the wssJ translated sequence). However, it is possible that translation may initiate at the first in-frame valine codon (amino acid residue number 1 in the wssJ translated sequence). In this case, the initiating amino acid of the protein product would still be methionine, not valine. Hence, the invention also provides a WssJ protein comprising the amino acid sequence from position 2 to position 324 of the amino acid sequence illustrated in FIG. 12 (SEQ ID NO: 11) but having an additional N-terminal methionine residue.

As discussed below, the products of the wss G, H and I genes do not form part of the polysaccharide synthase complex but are thought to be involved in modification of the glucan-like polymer.

In addition to wss operon, the inventors have identified a further novel operon in P. fluorescens, denoted the wsp operon, which encodes a chemotaxis-like operon of seven genes, wspA-F and wspR. The wspR gene product is involved in the regulation of the polysaccharide biosynthetic pathway of P. fluorescens. The wspR gene product has also been found to be involved in bacterial attachment and biofilm development in P. fluorescens. In addition, a wsp operon homologue has been shown to be involved in bacterial attachment in P. aeruginosa PA01.

Isolated individual genes from the Pseudomonas fluorescens wsp operon are also encompassed within the scope of the invention, as are constructs comprising combinations of two or more of the individual genes. Thus, the invention provides an isolated nucleic acid molecule encoding a WspA protein, said protein comprising the sequence of amino acids illustrated in SEQ ID NO: 28. Preferably the nucleic acid molecule comprises the sequence of nucleotides from position 4535 to position 6178 of the nucleic acid sequence illustrated in SEQ ID NO: 27.

Also provided by this aspect of the invention is a WspA protein comprising the sequence of amino acids illustrated SEQ ID NO: 28. The invention further provides a WspA protein encoded by a nucleic acid molecule according to the invention.

The amino acid sequence shown in SEQ ID NO: 28 is the predicted translation of the longest open reading frame of the Pseudomonas fluorescens wspA gene. Translation of the WspA protein in vivo is expected to initiate at the first possible in-frame methionine codon.

The invention also provides an isolated nucleic acid molecule encoding a WspB protein, said protein comprising the sequence of amino acids illustrated in SEQ ID NO: 29. Preferably the nucleic acid molecule comprises the sequence of nucleotides from position 6178 to position 6690 of the nucleic acid sequence illustrated in SEQ ID NO: 27.

Also provided by this aspect of the invention is a WspA protein comprising the sequence of amino acids illustrated SEQ ID NO: 29. The invention further provides a WspB protein encoded by a nucleic acid molecule according to the invention.

The amino acid sequence shown in SEQ ID NO: 29 is the predicted translation of the longest open reading frame of the Pseudomonas fluorescens wspB gene. Translation of the WspB protein in vivo is expected to initiate at the first possible in-frame methionine codon.

The invention also provides an isolated nucleic acid molecule encoding a WspC protein, said protein comprising the sequence of amino acids illustrated in SEQ ID NO: 30. Preferably the nucleic acid molecule comprises the sequence of nucleotides from position 6687 to position 7946 of the nucleic acid sequence illustrated in SEQ ID NO: 27.

Also provided by this aspect of the invention is a WspC protein comprising the sequence of amino acids illustrated SEQ ID NO: 30. The invention further provides a WspC protein encoded by a nucleic acid molecule according to the invention.

The amino acid sequence shown in SEQ ID NO: 30 is the predicted translation of the longest open reading frame of the Pseudomonas fluorescens wspC gene. Translation of the WspC protein in vivo is expected to initiate at the first possible in-frame methionine codon.

The invention provides an isolated nucleic acid molecule encoding a WspD protein, said protein comprising the sequence of amino acids illustrated in SEQ ID NO: 31. Preferably the nucleic acid molecule comprises the sequence of nucleotides from position 7943 to position 8641 of the nucleic acid sequence illustrated in SEQ ID NO: 27.

Also provided by this aspect of the invention is a WspD protein comprising the sequence of amino acids illustrated SEQ ID NO: 31. The invention further provides a WspD protein encoded by a nucleic acid molecule according to the invention.

The amino acid sequence shown in SEQ ID NO: 31 is the predicted translation of the longest open reading frame of the Pseudomonas fluorescens wspD gene. Translation of the WspD protein in vivo is expected to initiate at the first possible in-frame methionine codon.

The invention further provides an isolated nucleic acid molecule encoding a WspE protein, said protein comprising the sequence of amino acids illustrated in SEQ ID NO: 32. Preferably the nucleic acid molecule comprises the sequence of nucleotides from position 8638 to position 10905 of the nucleic acid sequence illustrated in SEQ ID NO: 27.

Also provided by this aspect of the invention is a WspE protein comprising the sequence of amino acids illustrated SEQ ID NO: 32. The invention further provides a WssA protein encoded by a nucleic acid molecule according to the invention.

The amino acid sequence shown in SEQ ID NO: 32 is the predicted translation of the longest open reading frame of the Pseudomonas fluorescens wspE gene. Translation of the WspE protein in vivo is expected to initiate at the first possible in-frame methionine codon.

The invention also provides an isolated nucleic acid molecule encoding a WspF protein, said protein comprising the sequence of amino acids illustrated in SEQ ID NO: 33. Preferably the nucleic acid molecule comprises the sequence of nucleotides from position 10902 to position 11912 of the nucleic acid sequence illustrated in SEQ ID NO: 27.

Also provided by this aspect of the invention is a WspF protein comprising the sequence of amino acids illustrated SEQ ID NO: 33. The invention further provides a WspF protein encoded by a nucleic acid molecule according to the invention.

The amino acid sequence shown in SEQ ID NO: 33 is the predicted translation of the longest open reading frame of the Pseudomonas fluorescens wspF gene. Translation of the WspF protein in vivo is expected to initiate at the first possible in-frame methionine codon.

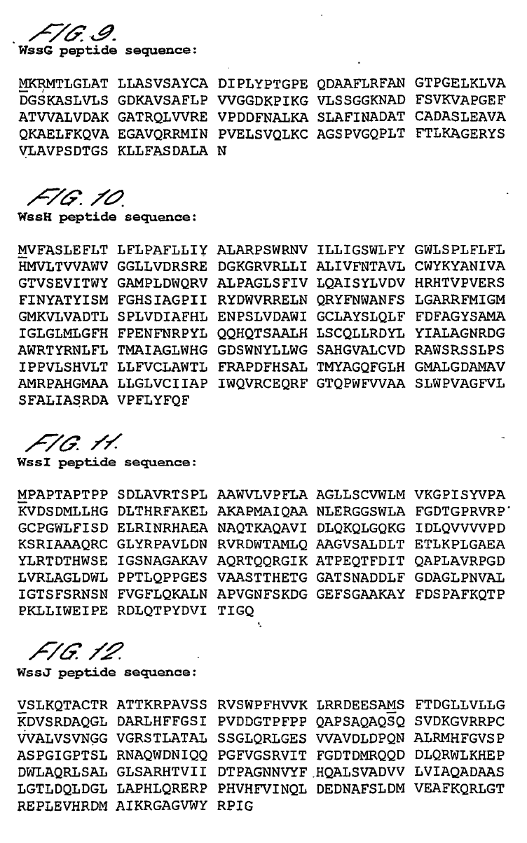

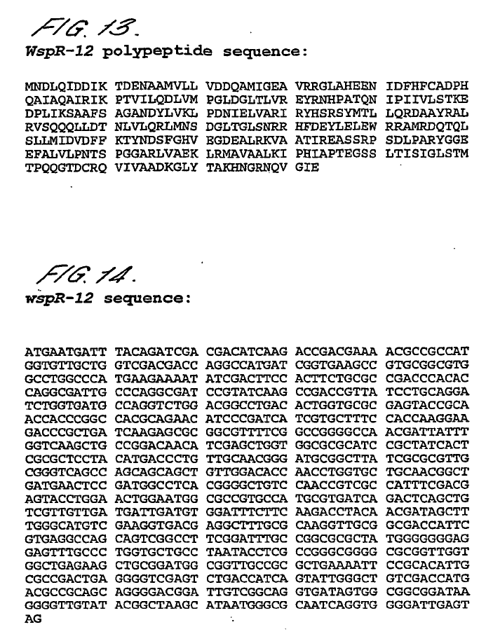

The invention still further provides an isolated nucleic acid molecule encoding a WspR protein, said protein comprising the sequence of amino acids illustrated in FIG. 13 (SEQ ID NO: 12). Preferably the nucleic acid molecule comprises the sequence of nucleotides illustrated in FIG. 14 (SEQ ID NO: 13).

Also provided by this aspect of the invention is a WspR protein having the sequence of amino acids illustrated in FIG. 13 (SEQ ID NO: 12). The invention further provides a WspR protein encoded by a nucleic acid molecule according to the invention.

The amino acid sequence shown in FIG. 13 (SEQ ID NO: 12) is the predicted translation of part of the longest open reading frame of the Pseudomonas fluorescens wspR gene. Translation of the WspR protein in vivo is predicted to initiate at the first possible in-frame methionine codon (amino acid residue number 1 in the wspR translated sequence).

The wspR protein plays a role in the regulation of the wss operon and is essential for the production of the cellulose-like polysaccharide. Evidence for this comes from the glucan-like polysaccharide defective phenotype of wspR mutants. WspR has two domains (N and C) and a linker region. The N-terminus is highly similar to the N-terminus found in response regulator proteins. The C-terminus is widespread among prokaryotes, with many genomes containing a large number of genes with this C-terminus. However, the function of this domain is unknown. The only similar gene for which a phenotype has been assigned is PleD, from Caulobacter cresentus which is a cell-cycle gene and is essential for flagella ejection. PleD differs from WspR in that it has a duplicated N-terminal domain.

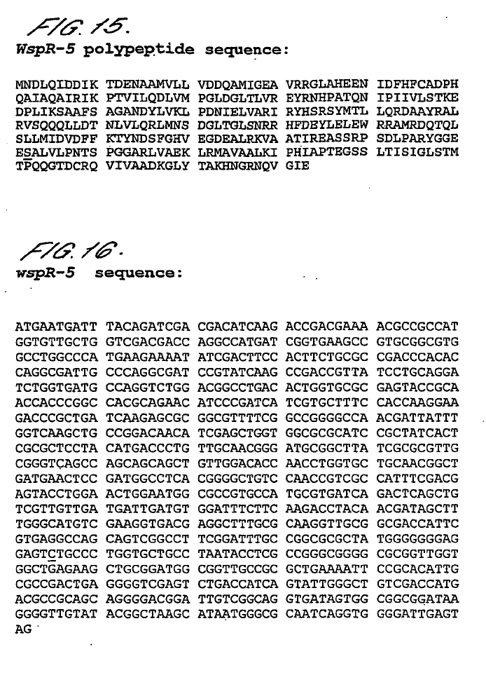

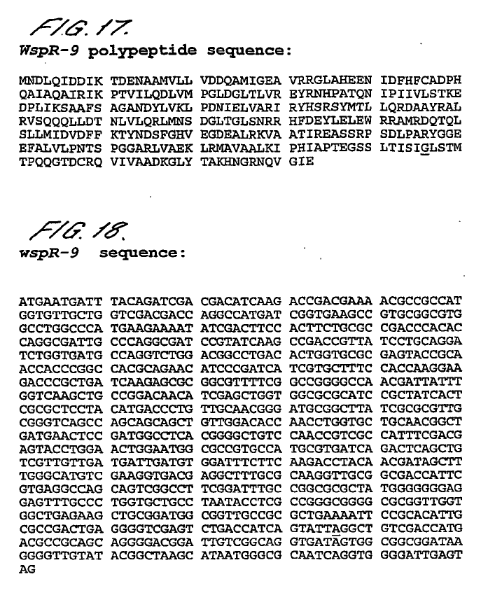

The wild-type wspR gene (allele), the coding sequence of which is shown in FIG. 14 (SEQ ID NO: 13), is also referred to as the wspR-12 allele. Additional variants of wspR have been sequenced and are given different allele numbers, as illustrated in the accompanying Figures. Therefore, in addition to the wild-type wspR, the invention further provides allelic variants of wspR comprising the nucleotide sequences shown in FIGS. 16 (wspR-5; SEQ ID NO:15), 18 (wspR-9; SEQ ID NO:17), 20 (wspR-13; SEQ ID NO:19), 22 (wspR-14; SEQ ID NO:21) and 24 (wspR-19; SEQ ID NO:23). The sequences illustrated in these Figures are the coding regions only of the wspR alleles, from the predicted initiation codon to the first in-frame stop codon. The invention also provides isolated WspR proteins encoded by each of the variant wspR alleles, the amino acid sequences of these proteins being illustrated in FIGS. 15, 17, 19, 21 and 23 (SEQ ID Nos:14, 16, 18, 20 and 22, respectively).

The invention also provides two isolated P. fluorescens genes which do not form part of the Wss operon but which may be involved in polysaccharide biosynthesis, specifically in chemical modification of glucan-like polymers, and also the protein products encoded thereby. These genes are designated mreB and pgi.

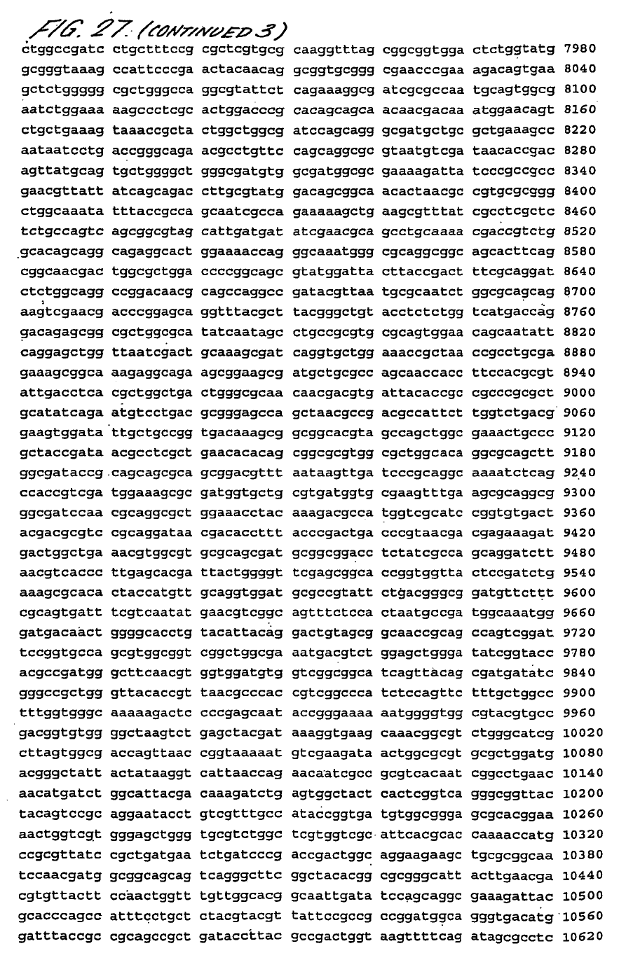

Thus, the invention provides an isolated nucleic acid molecule encoding a P. fluorescens phosphoglucose isomerase protein, which nucleic acid molecule comprises the nucleotide sequence illustrated in FIG. 25 (SEQ ID NO:24).

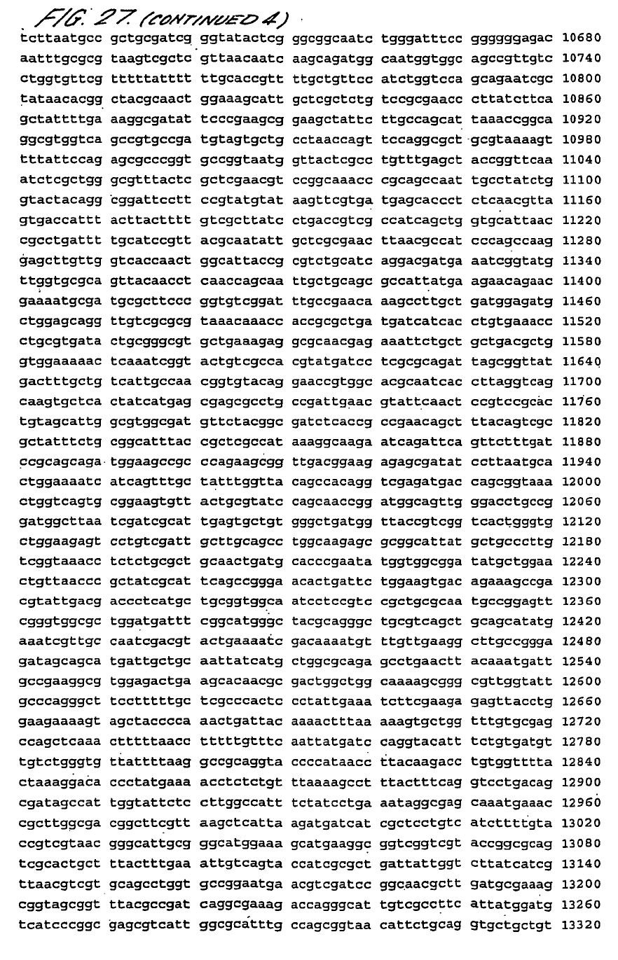

The invention further provides an isolated nucleic acid molecule comprising the nucleotide sequence illustrated in FIG. 26 (SEQ ID NO:25). The sequence shown in FIG. 26 (SEQ ID NO:25) covers a central region of the P. fluorescens mreB gene. A BLASTX search of genetic databases (via the BLAST server at www.ncbi.nlm.nih.gov/blast) identified the protein encoded from this region of DNA as being homologous to other sequenced mreB genes (e.g. E. coli mreB).

The involvement of mre and pgi in polysaccharide expression in the P. fluorescens SBW25 LSWS mutant was initially determined through the isolation of mini-Tn5 transposon mutants (WS-12, pgi mutant; WS-39, mre mutant). Subsequent sequence analysis allowed the mapping of the mini-Tn5 insertion site into the genome, and the identification of the disrupted gene. The mreB mutant has a SM-like colony morphology, i.e. is never wrinkly, and binds the Congo Red stain on all media. The pgi mutant binds congo red on LB plates, but not on KB and is wrinkly on LB but not on KB. Furthermore, the pgi mutant was unable to form biofilm mats in microcosm vials, and the mat formed by the mre mutant was significantly weaker than that formed by the LSWS ancestor.

The structure of the wss operon suggests that there are at least five genes products required for the expression of the glucan-like polysaccharide. Four of these genes (wssB-E) are required to form a polysaccharide synthase complex (based on homologies with the genes forming the cellulose synthase complex from Acetobacter xylinus; however, A. xylinus does not have a wssA homologue).

Phosphoglucose isomerase (PGI, pgi) is a highly conserved enzyme found throughout the prokaryota and eukaryota. Its role is the interconversion of glucose-6-phosphate and fructose-6-phosphate. This interconversion is a critical step in the glucogenic and glucolytic pathways. With respect to the growth of P. fluorescens in media containing glycerol as the main carbon source, PGI is needed to transfer some of the energy flowing into the Embden-Meyerhof (EM) pathway from the utilisation of glycerol into the production of glucose, a necessary intermediate for polysaccharide biosynthesis.

Because of the role of PGI in the production of intermediates for polysaccharide biosynthesis, the pgi gene may indirectly influence the efficiency and activity of the wss operon. It is thus a target for chemical mutation or other intervention as a means of indirectly influencing the activity of the wss operon.

The invention further provides a candidate glucan-like polysaccharide producing operon isolated from E. coli as well as individual genes therefrom and the protein products encoded by these genes.

The candidate E. coli polysaccharide producing operon provided by the invention comprises the nucleotide sequence illustrated in FIG. 27 (SEQ ID NO:26). The co-ordinates for the individual genes within the DNA fragment shown in FIG. 2 (SEQ ID NO:1) are as follows: yhjQ 336-1070 (wssA homologue); yhjO 995-3685 (wssB homologue); yhjN 3591-6035 (wssC homologue); yhjM 6036-7148 (wssD homologue); yhjL 7051-10602 (wssE homologue); yhjK 10627-12672; and yctA 12819-past the end of the given sequence.

Examination of the E. coli genome (via the public NCBI E. coli database, see: www.ncbi.nlm.nih.gov/cgi-bin/Entrez/framik?db=Genome&gi=115); identified an operon (yhj) which had five genes with strong homology to the wssA-E genes of the P. fluorescens polysaccharide biosynthetic operon. The yhj operon (yhjK-Q) is located at the 3,694,861-3,679,861 region of the E. coli genome. This region also includes dctA. At the time the inventors identified this operon there had been no previous publication reporting the expression of cellulose or a modified cellulose from this operon in E. coli. Nevertheless, by virtue of the fact that E. coli appeared to have the necessary genes for cellulose expression, the inventors decided to test whether a polysaccharide-producing mutant strain could be generated using the microcosm system used to generate a P. fluorescens strain that expressed a glucan-like polysaccharide. Subsequently, a WS-like E. coli DH5A mutant was isolated which takes up Congo Red stain on agar plates, and which binds the cellulose specific Calcoflour stain. These initial tests strongly support the suggestion that this strain of E. coli now expresses polysaccharide in a similar manner to the LSWS strain of P. fluorescens SBW25. Zogaj et al. (Zogaj, X., Nimtz, M., Rohde, M., Bokranz, W., and Romling, U. (2001). The multicellular morphotypes of Salmonella typhimurium and Escherichia coli produce cellulose as the second component of the extracellular matrix. Mol. Microbiol. 39: 1452-1463) have subsequently also described the expression of cellulose in E. coli.

In accordance with the present invention, a defined nucleic acid includes not only the identical nucleic acid but also any minor base variations, including in particular base substitutions which result in a synonymous codon (a different codon specifying the same amino acid residue) due to the degeneracy of the genetic code. The term “nucleic acid” includes single or double stranded RNA, single or double stranded DNA, synthetic forms and mixed polymers, both sense and antisense strands, and may be chemically or biochemically modified or may contain non-natural or derivatized nucleotide bases as will be readily appreciated by those skilled in the art. Such modifications include, for example, labels, methylation, substitution of one or more naturally occurring nucleotide bases with an analog. Libraries of bacterial chromosomal fragments may be screened as natural sources of the nucleic acids of the present invention. Alternatively, nucleic acid sequences according to the invention may be produced using recombinant or synthetic means, for example by PCR amplification of sequences resident in chromosomal DNA or cloned fragments thereof. Generally such techniques are well known in the art (see Sambrook et al. (1989), Molecular Cloning: A Laboratory Manual, Cold Spring Harbor Laboratory Press; F. M. Ausubel et al. (eds.), Current Protocols in Molecular Biology, John Wiley & Sons, Inc. (1994)).

The DNA molecules according to the invention may, advantageously, be included in a suitable expression vector to express the protein encoded therefrom in a suitable host. Procedures for incorporation of a cloned DNA into a suitable expression vector, transformation of a host cell and subsequent selection of the transformed cells are well known to those skilled in the art, as provided by Sambrook et al. (1989), Molecular Cloning: A Laboratory Manual, Cold Spring Harbor Laboratory Press or F. M. Ausubel et al. (eds.), Current Protocols in Molecular Biology, John Wiley & Sons, Inc. (1994).

An expression vector according to the invention includes a vector comprising a nucleic acid molecule according to the invention operably linked to regulatory sequences, such as promoter regions, that are capable of effecting expression of the said nucleic acid. The term “operably linked” refers to a juxtaposition wherein the components described are in a relationship permitting them to function in their intended manner. Such vectors may be introduced into a suitable host cell to provide for expression of a polypeptide according to the invention. Thus, in a further aspect, the invention provides a process for preparing polypeptides according to the invention which comprises cultivating a host cell, comprising an expression vector as described above under conditions to provide for expression by the vector of a coding sequence encoding the polypeptides, and recovering the expressed polypeptides.

Expression vectors suitable for driving expression of a given protein in a prokaryotic host cell may be, for example, plasmid or phage vectors provided with an origin of replication, and a promoter to drive transcription of mRNA encoding the said protein and optionally a regulator of the promoter. The vectors may contain one or more selectable markers, such as, for example, ampicillin resistance.

Sequence elements required for prokaryotic expression include promoter sequences to bind RNA polymerase and processing information sites such as ribosome binding sites, transcription termination sites etc. For example, a bacterial expression vector may include a promoter to bind RNA polymerase and direct an appropriate frequency of transcription initiation at the transcription start site and for translation initiation the Shine-Dalgarno sequence and a translation initiation codon (usually AUG). The promoter may be the promoter naturally associated with the coding region in question or may be a heterologous promoter such as, for example, the lac promoter. In a preferred embodiment the promoter is inducible, being activated by binding of an appropriate transcriptional regulatory molecule or promoter-specific RNA polymerase. Expression of the target protein can thus be temporally controlled by controlling expression and/or activation of the transcriptional regulatory molecule or promoter-specific RNA polymerase. Well known examples of such inducible systems are the T7 promoter/T7polymerase, T3 promoter/T3 polymerase and SP6 promoter/SP6 polymerase systems and the lacUV5 promoter/IPTG system. Vectors suitable for expression in a range of prokaryotic hosts may be obtained commercially or assembled from the sequences described by methods well known in the art.

A further aspect the invention also provides a host cell or organism comprising an expression vector according to the invention. Preferably, the host cell/organism is a prokaryotic cell/organism.

In accordance with the present invention, a defined protein or polypeptide includes proteins which are substantially homologous but have one or more conservative amino acid changes, including naturally occurring allelic variants, or in vivo or in vitro chemical or biochemical modifications (e.g. acetylation, carboxylation, phosphorylation, glycosylation etc). In this context, a “substantially homologous” sequence is regarded as a sequence which shares at least 80%, preferably at least 90% and more preferably at least 95% amino acid sequence identity with the proteins or polypeptides of the invention. The protein according to the invention may be recombinant, synthetic or naturally occurring, but is preferably recombinant.

Also within the scope of the invention are fusion proteins/polypeptides comprising a protein according to the invention. The proteins of the invention may be fused either N-terminally or C-terminally to heterologous protein or peptide fragments, for example to facilitate purification of the fusion protein. Fusion proteins will typically be made by recombinant nucleic acid techniques or may be chemically synthesized.

The invention also provides for cleavage fragments of the full length proteins, particularly cleavage fragments of the enzymes which share homology with cellulose synthase subunits. It is not uncommon for bacterial genes to encode a inactive precursor form of an enzyme/enzyme subunit which is post-translationally processed to yield shorter polypeptides which participate in catalytic and/or regulatory activity of the enzyme/enzyme complex.

In a still further aspect, the invention provides a method of constructing an exopolysaccharide-producing bacterial strain, which method comprises introducing an expression vector suitable for overexpression of a WspR protein into a host bacterial strain, the genome of which contains a wss-like operon.

Advantageously, the host bacterial strain may be a Pseudomonas strain or an E. coli strain. Preferred host strains include wild type Pseudomonas fluorescens strain SBW25 and E. coli strain K12.

In one embodiment, the WspR protein is a wild-type WspR protein comprising the amino acid sequence illustrated in FIG. 13 (SEQ ID NO:12), in which case optimum production of polysaccharide from the resultant exopolysaccharide-producing strain may require the addition of NaCl to the culture medium (see accompanying Examples). In this embodiment, the expression vector may conveniently comprise the sequence of nucleotides shown in FIG. 14 (SEQ ID NO:13) operably linked to sequences which control its expression.

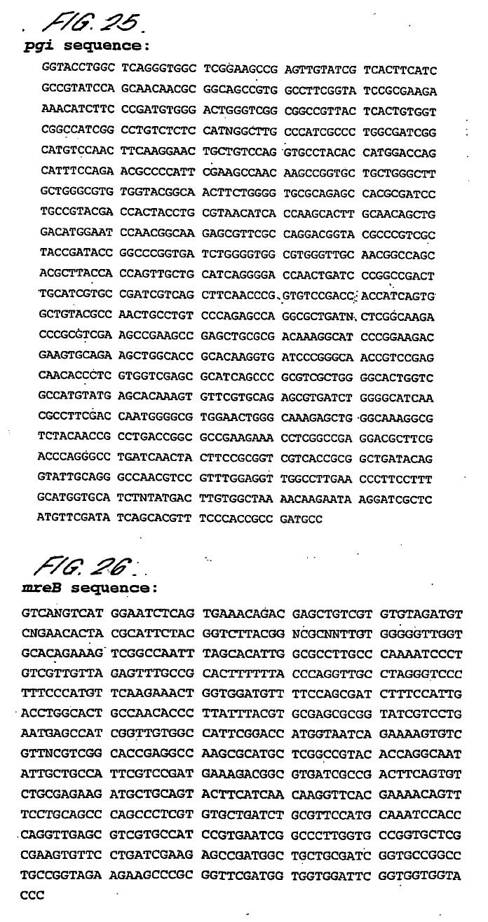

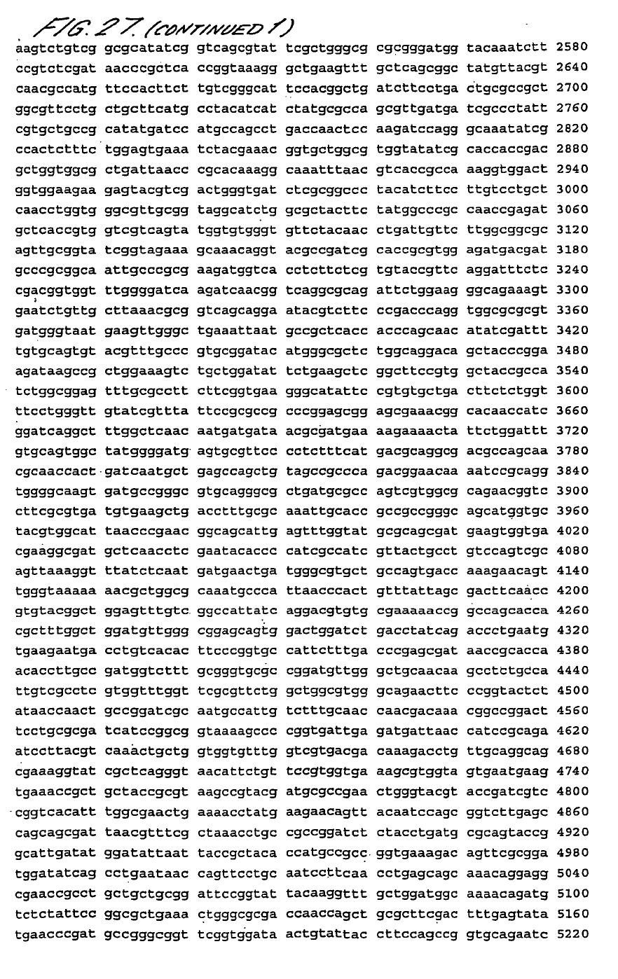

In a further embodiment, the WspR protein is an allelic variant of the Pseudomonas fluorescens WspR protein having the amino acid sequence illustrated in FIG. 21 (WspR-14; SEQ ID NO:20) or in FIG. 23 (WspR-19; SEQ ID NO:22). Exopolysaccharide-producing strains expressing these variant WspR proteins generally do not require the presence of additional NaCl for optimal polysaccharide production (see accompanying Examples).

For this aspect of the invention, it may be useful to construct an expression vector in which nucleic acid encoding the wspR protein is placed under the control of an inducible promoter (see above). Host cells containing such a construct can be grown up in culture with wspR expression, and hence β-linked glucan production, switched off then at the appropriate time expression of wspR can be induced, leading to expression of the cellulose biosynthetic enzymes.

Also included within the scope of the invention is an embodiment in which the expression vector is an invasive plasmid which can be used to transform a host bacterium in situ, meaning in the field or in the natural environment of the bacterium as opposed to in in vitro culture in a laboratory.

A particularly useful application of this method of the invention is in the introduction of a WspR expressing plasmid into a soil-dwelling bacterium. Switching on glucan-like polysaccharide production in such a bacterium may enable the bacterium to stick to and colonise the roots of a plant or may render the bacterium more resistant to dessication. Cellulose production is known in a number of bacteria, but has received attention only in Acetobacter (where it has been studied from the point of view of cellulose extraction) and Agrobacterium. Studies in Agrobacterium have focussed on its role in attachment to the plant surface. The data is not entirely clear, but it does appear to have a role. The inventors postulate that the ecological role of the glucan-like polysaccharide in P. fluorescens may be in surface colonisation rather than attachment. It is proposed that the wild-type bacterium can regulate (through the wsp (chemosensory) operon) the amount of cellulose produced from the poles of the cells and can thereby control whether cells spread rapidly across a surface in a thin later, or pile up in a mound. The ability to control surface colonisation may have considerable biotechnological potential.

The invention further provides an exopolysaccharide-producing strain which is obtainable by the above-described methods. In a preferred embodiment, the exopolysaccharide-producing strain, in addition to expression of a WspR protein, further carries a mutation in the mreB gene, the pgi gene or both the aforementioned genes.

As explained herein, both the mreB gene product and the pgi gene product of Pseudomonas fluorescens, and hence homologous genes from other bacterial strains, may influence the nature of the polysaccharide product produced by the action of enzymes encoded by the wss operon. Hence, strains which carry a mutation in either or both of these genes may produce varying polysaccharide products.

The wspR protein also plays a role in the attachment of bacteria to the sides and surfaces of culture containers. Bacterial attachment is the first stage in the development of a biofilm. Subsequent biofilm development proceeds from the attached cells out into the liquid media and new bacterial remain connected to the attached cells via the expression of exocellular polysaccharide or proteinaceous matrix or skeleton. Evidence for this in P. fluorescens comes from a comparison of attachment abilities of various wrinkley spreader mutants. The original wrinkly spreader strain (WS) and the mutant strain WS-13 (unable to express glucan-like polysaccharide) are able to attach readily to the surfaces of culture containers. In contrast, WS-4 (wspR⁻) is unable to attach to surfaces at all. Similarily, a wrinkly spreader strain with a wspΔ (WS wspΔ) is not able to attach either. Futhermore, like P. fluorsecens WS wspΔ, the inventors have found that P. aeruginosa PA01 deleted for the wsp-like operon, is also defficient in bacterial attachment.

A particularly useful application of the invention may be found in the removal of a biofilm. The production of extracellular cellulose plays an important part in the development of biofilms. The inventors have observed that expression of the WspR allelic variants WspR-5, WspR-9 and WspR-13 in a strain of Pseudomonas fluorescens which is producing glucan-like polysaccharide (i e having wrinkly spreader morphology) results in cessation of polysaccharide production and return to an SM phenotype. It is therefore to be expected that introduction of an expression vector encoding wspR-5, wspR-9 or wspR-13 into bacteria which make up a biofilm would shut down polysaccharide production and hence promote disruption of the biofilm. Again, this would be advantageously achieved using an invasive plasmid encoding wspR-5, wspR-9 or wspR-13 to transform the biofilm in situ. This approach might be adapted for use in essentially any situation where formation of biofilms is known to be a problem. A particular example from industry is in the paper making process where a number of rolling and screening stages are used, each stage being subject to bio-fouling, often associated with formation of a biofilm. Currently this problem is addressed with the use of anti-microbial agents.

In addition, the identification of the role of WspR in bacterial attachment and biofilm development provides a further application of appropriately modified P. fluorescens or P. aeruginosa strains in which bacetrial attachment is used to screen chemical and pharmaceutical libraries for compounds that inhibit attachment and biofilm growth. These compounds might directly prevent cellulose production, directly prevent normal WspR function, or prevent WspR function indirectly. The compounds may bind specifically with WspR preventing normal wspR interactions with other cellular components involved in cellulose biosythesis, attachment or biofilm development. Alternatively, these compounds may interfere with the production or function of cellular components which act on, or with WspR for normal function. The screening system can also be used to identify compounds inhibiting cellulose biosythesis, attachment or biofilm development without interacting directly with WspR.

Screening Assays for Inhibitors of Exopolysaccharide Production, Bacterial Attachment and Biofilm Development.

Using P. fluorescens SBW25 and P. aeruginosa PA01, the present inventors have discovered that the wsp operon plays an important role in the attachment of bacterial cells to solid surfaces. Further, it has been determined that the development of biofilms first requires bacterial attachment. Biofilms are of particular problem in human medicine, where high concentrations of bacteria can exist in tissues and be protected by a secreted, biofilm matrix or scaffold. The inventors' observations lead to the conclusion that in P. fluorescens, P. aeruginosa and other pseudomonads bacterial attachment is regulated by wsp, or a wsp-like operon of speacilised chemotatic or cheomosensory genes. Some environmental or internal signal is received by this system, and the signal is passed down to WspR, or a WspR-like protein. The activated WspR then stimulates the expression of downstream genes required for the actual physical attachment of bacteria.

The observation that the wsp operon is involved in bacterial attachment and biofilm development has led to the development of assays which may be used to screen libraries of compounds to identify inhibitors of expolysaccharide production, bacterial attachment and biofilm development. In essence, these assays rely on the production of exopolysaccharide by wrinkly spreader (WS) strains or modified SM strains.

In P. fluorescens, attachment and activated WspR, also stimulate the expression of exopolysaccharide leading to the development of a biofilm. In P. aeruginosa PA01, no cellulose biosynthetic genes exist but it is possible that activated WspR may induce other biofilm-forming genes, such as alignate biosythesis.

A number of assays are provided which may be used to screen chemical libraries for compounds that inhibit bacterial attachment and biofilm development:

Assay for Exopolysaccharide Production:

In this assay exopolysaccharide-producing bacteria are incubated in the presence of test chemicals on agar plates containing a dye which specifically stains the exopolysaccharide, for example Congo Red. Production of the exopolysaccharide is scored by observing uptake of the dye.

Assay for attachment and biofilm Production:

In this assay exopolysaccharide-producing bacteria are incubated in the presence of test chemicals in liquid broth culture. Attachment and biofilm development is scored by visual inspection, as described in the accompanying examples. Crystal violet staining may also be used to provide quantitive results for bacterial attachment.

Advantageously, the attachment assay may be carried out in microtitre assay plates where 96 or more individual cultures can be tested on a single plate. The testing of different culture containers is important in assay optimisation, as some attachment systems are affected by different materials (P. fluorescens SBW25 will attach easily to glass and polystyrene).

The exopolysaccharide production assay and the assay for attachment and biofilm formation may both be performed using any of the exopolysaccharide producing bacterial strains described herein, including evolved variant strains having wrinkly spreader morphology, recombinant strains, mutagenized strains etc.

The assays can also be perfomed using modified forms of the wild-type P. fluorescens SBW25 which have been engineered to express either wpR14 or wspR19. The wild-type SBW25 strain contains the wild-type wspR12 allele and is phenotypically smooth (SM). When modified to express wspR14 or wspR19 it exhibits the wrinkly spreader phenotype, produces exopolysaccharide and forms biofilms. In contrast, if a wrinkly spreader strain is engineered to wspR5, 9 or 13 it will exhibit a smooth (SM) phenotype. These alterations can be used to manipulate the assay an enable screening on specific allelic forms of wspR, for example a wrinkly spreader strain engineered to express wspR5 (now phenotypically SM) will produce exopolysaccharide if the test chemical interferes with the wspR5 protein but not the chromosomal copy of wspR12.

A liquid culture based assay for inhibitors of attachment may also be carried out using a bacterial strain which expresses the gene-products of a wsp-like operon and a control strain which is essentially identical but which does not expresses the gene-products of the wsp-like operon. The inventors have shown by experiment that Wsp homologues are required for attachment in the liquid culture system. Thus, the assay can be carried out using essentially any bacterial strain which expresses the Wsp homologs required for attachment. Specificity is provided by the use of the control strain which does not express the Wsp homologues. In this context the term “Wsp homologues” encompasses proteins which exhibit at least 75% sequence similarlity with the homologous P. fluorescens Wsp protein and/or the homologous P. aeruginosa Wsp protein at the amino acid level.

“Wsp-like operon” is a generic term used herein to describe a novel class of bacterial operons containing genes which encode proteins involved in the regulation of glucan-like polysaccharides synthesis, in bacterial attachment and/or biofilm formation. Encompassed within the term “wsp-like operon” are operons which are homologous to the wsp operon of Pseudomonas fluorescens, the complete nucleotide sequence of which is given herein. Preferably, the wsp-like operon comprises a sequence of nucleotides which shares at least 50% nucleotide sequence identity with the sequence of nucleotides shown in SEQ ID NO: 27. Even more preferably, the wss-like operon may comprise a sequence of nucleotides at least 55%, at least 60%, at least 65%, at least 70%, at least 75%, at least 80%, at least 90% or at least 95% identical to the P. fluorescens wsp operon shown in SEQ ID NO: 27. Percentage nucleotide identity may be calculated by comparing entire wsp operon sequences or by comparing the coding regions of the individual genes of the operons. In the latter case, the coding regions of homologous genes should be compared. A value for the overall percentage sequence identity may then be derived by taking an average over all the individual homologous coding regions. A complete annotation of the P. fluorescens wsp operon, showing the positions of the coding regions is listed elsewhere in this specification.

In a preferred embodiment the assay may be based on the use of P. aeruginosa strain PA01 (a well-characterised strain which is available from public strain collections, see Holloway, B. W. (1955). Genetic recombination in Pseudomonas aeruginosa. J. Gen. Microbiol. 13, 572-581; Stover, C. K., Pham, X. Q., Erwin, A. L., et al. (2000). Complete genome sequence of Pseudomonas aeruginosa PA01, an opportunistic pathogen. Nature 406: 959-964) in order to screen for chemicals which specifically interfere with PA01 attachment. By using wild type PA01 and PA01 wspΔ (i.e. PA01 with the wsp operon deleted) it is possibly to identify chemicals which specifically interfere with attachment of P. aeruginosa. Experiments have shown that the PA01 wsp operon homologue is required for attachment in the liquid culture system, thus PA01 wspΔ should always grow in liquid culture unless the test chemicals are toxic but does not attach, whereas wild type PA01 should attach unless the test chemical has an effect on the attachment process.

In a further preferred embodiment the attachment assay may be carried out using wild-type P. fluorescens and an equivalent strain deleted for the wsp operon, e.g. wild-type P. fluorescens SBW25 and P. fluorescens SBW25 wspΔ.

A wide variety of candidate chemicals may be tested in the screening methods of the invention. Suitable test chemicals may include, for example, chemicals having a known biochemical activity, chemicals having no such identified activity and completely new molecules or libraries of molecules such as might be generated by combinatorial chemistry. Test chemicals which are nucleic acids, including naturally occuring nucleic acids and synthetic analogues, polypeptides or proteins are not excluded.

Typically, screening assays involve running a plurality of assay mixtures in parallel with different concentrations of the test chemical. Typically, one of these concentrations serves as a negative control, i.e. zero concentration of test chemical.

Construction of Exopolysaccharide-Producing Strains Based on Expression of wss Gene Products

In a further aspect, the invention provides exopolysaccharide-producing bacterial strains based on expression of the wss operon.

It will be readily appreciated by a skilled artisan that it is possible to genetically modify a bacterium which does not naturally produce extracellular polysaccharide to make an exopolysaccharide-producing strain by introducing an expression vector suitable for driving expression of protein products of the wss operon which are required for polysaccharide biosynthesis.

Accordingly, the invention provides an exopolysaccharide-producing bacterial strain which is a bacterial host strain containing an expression vector including a nucleic acid comprising coding regions of a wss-like operon operably linked to regulatory sequences which control expression of the said nucleic acid.

In one embodiment, the expression vector may comprise a nucleic acid comprising all the coding regions of the Pseudomonas fluorescens wss operon, or all the coding regions of the E. coli yhj operon operably linked to appropriate expression regulatory sequences, or just the coding regions which are absolutely essential for polysaccharide biosynthesis. It will be appreciated that the expression vector may also contain intergenic regions from the wss operon in question in addition to the coding regions. It will further be appreciated that exopolysaccharide producing strains could be produced by co-expressing wss gene products from different species/strains. By combining cellulase synthase subunits from different species/strains in this manner it may be possible to alter the specificity of the enzyme and hence alter the structure of the resultant polysaccharide.

The expression regulatory sequences may comprise a promoter which is constitutively active in the bacterial host cell in question, leading to constitutive expression of the wss proteins or, in an alternative embodiment, an inducible promoter to enable polysaccharide production to be regulated.

In one embodiment, the expression regulatory sequences comprise the “authentic” promoter region of the wss operon. For example, an expression vector containing coding regions from the P. fluorescens wss operon would contain the promoter region of the P. fluorescens wss operon. In one embodiment, the expression vector might comprise a substantially complete wss operon, including the promoter region and all of the enzyme-encoding genes.

For those embodiments wherein the expression vector comprises an authentic wss promoter, the host bacterial strain should also comprise nucleic acid encoding a WspR protein which functions as a regulator of the wss promoter. Advantageously, the nucleic acid encoding the WspR protein may be present on a second expression vector under the control of a constitutive or inducible promoter, as appropriate.

The wss operon contains several more genes than would, on the basis of homology with cellulose biosynthetic operons from other bacterial species, seem to be required for the production of ‘pure’ cellulose. Three genes at the end of the operon (wssG, wssH, wssI) show similarity to the P. aeruginosa genes, aglF, algI & algJ. In P. aeruginosa, these three genes are responsible for acetylation of mannose residues that are part of the alginate polymer. The similarity between these genes and those in P. fluorescens is low, nonetheless, their presence suggests that a basic glucan-like polymer may be modified, possibly by acetylation. Mutants in wssGHI have been isolated that have a different phenotype on agar plates (no longer wrinkly) and lack ability to colonise the air-liquid interface in static microcosms, and yet still produce a polysaccharide that stains positively with calcofluor and Congo red. Thus, there exist within the wss operon genes that affect the nature of the basic glucan-like polymer. This opens the door to the construction of novel glucan-like polysaccharides. Moreover, further genes which influence the nature of the glucan-like polymer may be found outside of the wss operon, a particular example being the pgi gene discussed previously. It may thus be possible to construct further novel glucan-like polysaccharides by manipulation of genes outside the wss operon, by mutation or other means.

Process for the Production of Glucan-Like Polysaccharide

In a further aspect the invention also provides a process for the production of glucan-like polysaccharide from an exopolysaccharide-producing bacterial strain, which process comprises the steps of growing an exopolysaccharide-producing bacterial strain according to the invention and isolating the polysaccharide produced thereby.

The exopolysaccharide-producing bacterial strain can be any such strain described herein, including evolved variant strains, engineered strains, mutant strains etc.

In a preferred embodiment, the step of isolating the polysaccharide comprises lysing the bacteria in a bacterial lysis solution, for example 20 mM Tris HCl pH 8.0, 5 mM MgCl₂, 0.5% Sarkosyl, 1 mg/ml fresh lysozyme and incubating the sample thus obtained in with a second lysis solution comprising detergent and Proteinase K, for example 500 mM EDTA pH 9.0, 1% Sarkosyl, 1.5 mg/ml Proteinase K.

As discussed above, exopolysaccharide-producing evolved variants of Pseudomonas, for example evolved variants of Pseudomonas fluorescens, have a characteristic wrinkly spreader morphology, as described by Rainey & Travisano, 1998, Nature, 394: 69-72. Cells of the WS morph adhere firmly to each other and to surfaces, allowing the formation of a self-supporting mat at the air-broth interface when grown in a standard microcosm (see Example 1). The WS morph can also be grown on hard agar plates to form single colonies. Polysaccharide can be isolated from cells grown in either type of culture, colonies or mats, using substantially the same protocol for polysaccharide purification (see Example 2).

It will be appreciated that the precise composition of the polysaccharide product produced by a given exopolysaccharide-producing strain may vary slightly according to the type of carbohydrate added to the culture media in which the bacteria are grown, as this will ultimately determine the nature of the substrate available for the polysaccharide biosynthetic enzymes. Accordingly, it is within the scope of the invention to vary the precise composition of the polysaccharide by manipulating the type of carbohydrate added to the culture medium.

The present invention will be further understood with reference to the following non-limiting Examples, together with the accompanying Figures in which:

FIG. 1 is a schematic representation of the wss operon. The operon consists of ten genes (wssA-J) located on a ˜20 kb fragment of Pseudomonas fluorescens SBW25 genomic DNA. Some restriction sites are indicated below the coding regions; B, BamHI; H, HindIII and K, KpnI. The scale is given in 1 kb units.

FIG. 2 shows the nucleotide sequence of a contiguous 20,306 bp fragment of genomic DNA from Pseudomonas fluorescens SBW25. The wss operon, encoding the cellulose biosynthetic genes and associated genes, is located approximately between 2,200-18,000 bp.

FIG. 3 illustrates the predicted translation of the longest open reading frame of the P. fluorescens wssA gene. The sequence shown is from the first potential start codon (GTG; V valine) to the first in-frame stop codon. In order to easily place the peptide sequence within the DNA sequence of the wss operon the potential GTG-encoded start codon has been left as ‘V’ in the peptide sequence even though the initiating amino acid would in fact be methionine. The first potential ATG start codon (M) is also marked.

FIG. 4 illustrates the predicted translation of the longest open reading frame of the P. fluorescens wssB gene, from the first potential ATG start codon to the first in-frame stop codon.

FIG. 5 illustrates the predicted translation of the longest open reading frame of the P. fluorescens wssC gene, from the first potential GTG start codon (V) to the first in-frame stop codon. The first potential ATG start codon is also marked (M).

FIG. 6 illustrates the predicted translation of the longest open reading frame of the P. fluorescens wssD gene, from the first potential GTG start codon (V) to the first in-frame stop codon. The first potential ATG start codon is also marked (M).

FIG. 7 illustrates the predicted translation of the longest open reading frame of the P. fluorescens wssE gene, from the first potential ATG start codon (M) to the first in-frame stop codon.

FIG. 8 illustrates the predicted translation of the longest open reading frame of the P. fluorescens wssF gene, from the first potential ATG start codon (M) to the first in-frame stop codon.

FIG. 9 illustrates the predicted translation of the longest open reading frame of the P. fluorescens wssG gene, from the first potential ATG start codon (M) to the first in-frame stop codon.

FIG. 10 illustrates the predicted translation of the longest open reading frame of the P. fluorescens wssH gene, from the first potential ATG start codon (M) to the first in-frame stop codon.

FIG. 11 illustrates the predicted translation of the longest open reading frame of the P. fluorescens wssI gene, from the first potential ATG start codon (M) to the first in-frame stop codon.

FIG. 12 illustrates the predicted translation of the longest open reading frame of the P. fluorescens wssJ gene, from the first potential ATG start codon (M) to the first in-frame stop codon.

FIG. 13 illustrates the predicted translation of the longest open reading frame of the wild-type P. fluorescens wspR gene (allele WspR-12).

FIG. 14 shows the nucleotide sequence of the coding region of the wild-type P. fluorescens wspR gene (allele WspR-12) from the first potential ATG start codon to the first in-frame stop codon TAG.

FIG. 15 illustrates the predicted translation of the longest open reading frame of the variant P. fluorescens wspR allele WspR-5.

FIG. 16 shows the nucleotide sequence of the coding region of the variant P. fluorescens wspr allele WspR-5 from the first potential ATG start codon to the first in-frame stop codon TAG.

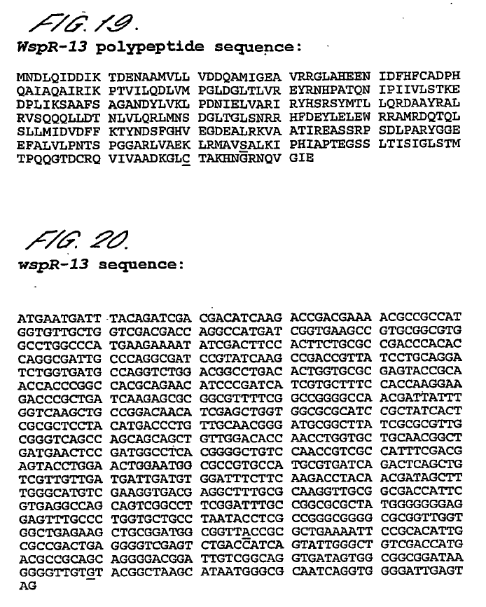

FIG. 17 illustrates the predicted translation of the longest open reading frame of the variant P. fluorescens wspR allele WspR-9.

FIG. 18 shows the nucleotide sequence of the coding region of the variant P. fluorescens wspR allele WspR-9 from the first potential ATG start codon to the first in-frame stop codon TAG.

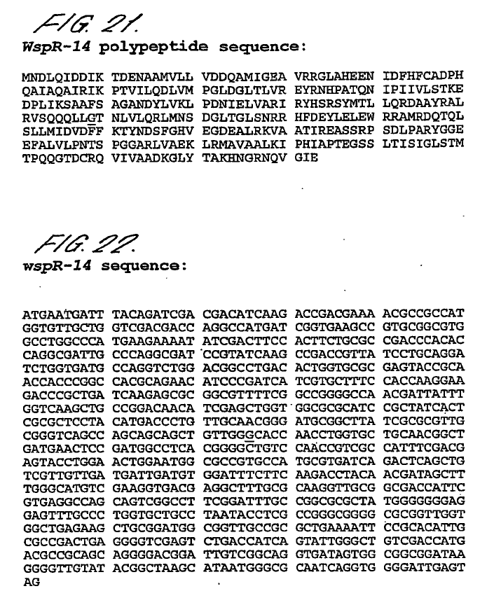

FIG. 19 illustrates the predicted translation of the longest open reading frame of the variant P. fluorescens wspR allele WspR-13.

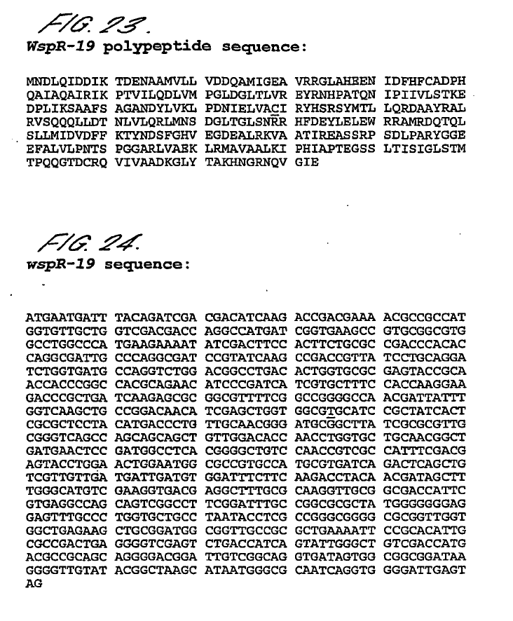

FIG. 20 shows the nucleotide sequence of the coding region of the variant P. fluorescens wspr allele WspR-13 from the first potential ATG start codon to the first in-frame stop codon TAG.

FIG. 21 illustrates the predicted translation of the longest open reading frame of the variant P. fluorescens wspR allele WspR-14.

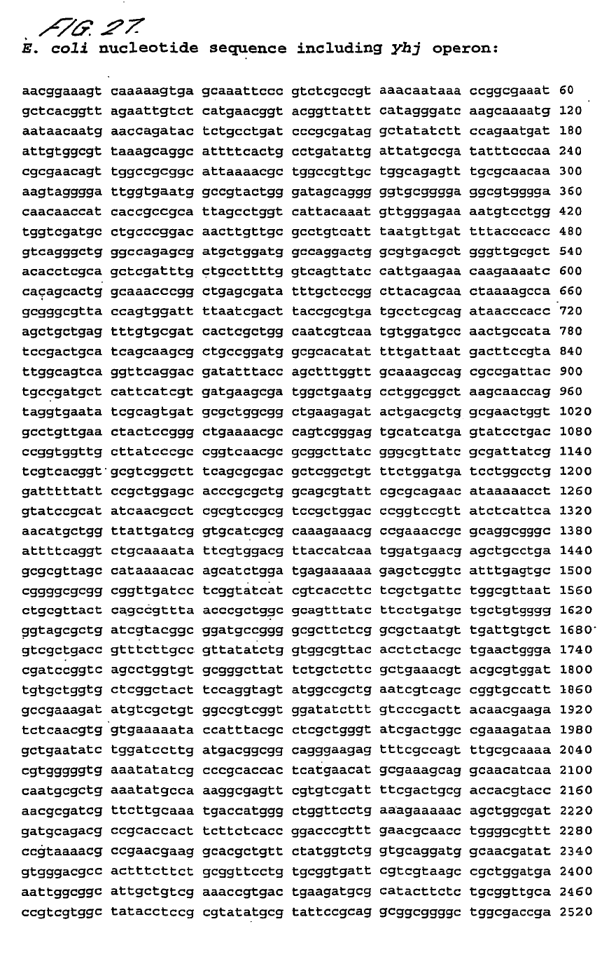

FIG. 22 shows the nucleotide sequence of the coding region of the variant P. fluorescens wspR allele WspR-14 from the first potential ATG start codon to the first in-frame stop codon TAG.

FIG. 23 illustrates the predicted translation of the longest open reading frame of the variant P. fluorescens wspR allele WspR-19.

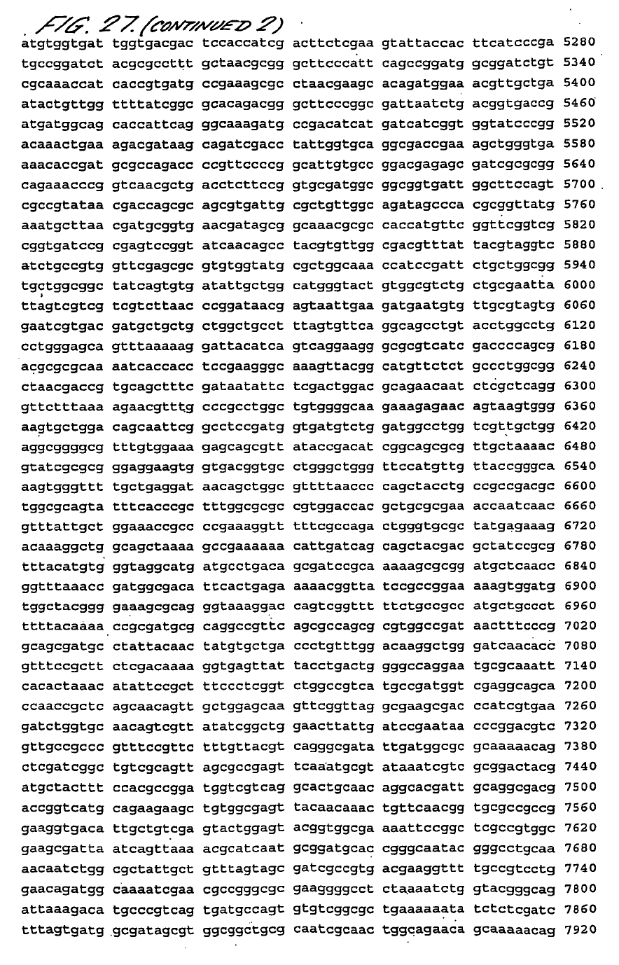

FIG. 24 shows the nucleotide sequence of the coding region of the variant P. fluorescens wspR allele WspR-19 from the first potential ATG start codon to the first in-frame stop codon TAG.

FIG. 25 shows the nucleotide sequence of a near-contiguous piece of DNA of 1,136 bp from the genome of Pseudomonas fluorescens SBW25. The sequence covers a central region of the pgi (phosphoglucose isomerase) gene. A BLASTX search of genetic databases (via the BLAST server at www ncbi nlm nih gov/blast) identified the protein encoded from this region of DNA as being homologous to other sequenced pgi genes (e.g. E. coli pgi).

FIG. 26 shows the nucleotide sequence of a near-contiguous piece of DNA of 703 bp from the genome of Pseudomonas fluorescens SBW25. The sequence covers a central region of the mreB (murien biosynthesis B) gene. A BLASTX search of genetic databases (via the BLAST server at www ncbi nlm nih gov/blast) identified the protein encoded from this region of DNA as being homologous to other sequenced mreb genes (e.g. E. coli mreB).

FIG. 27 shows the nucleotide sequence for a contiguous piece of DNA of 14,000 bp from the genome of Escherichia coli. The DNA sequence described here has been obtained from the public NCBI E. coli database (see: www ncbi.nlm.nih.gov/cgi-bin/Entrez/framik?db=Genome&gi=115); the 14,000 bp comes from the 3,694,861-3,679,861 region of genome. This region includes the yhj operon (yhjK-Q) and also includes dctA. The co-ordinates for the coding regions of the yhj genes can be found at the web site given above; the co-ordinates for the genes for the DNA segment shown in this Figure are: (any codon start)

yhjQ 336-1070 (wssA homologue);

yhjO 995-3685 (wssB homologue);

yhjN 3591-6035 (wssC homologue);

yhjM 6036-7148 (wssD homologue);

yhjL 7051-10602 (wssE homologue);

yhjK 10627-12672; and

yctA 12819-past the end of the given sequence.

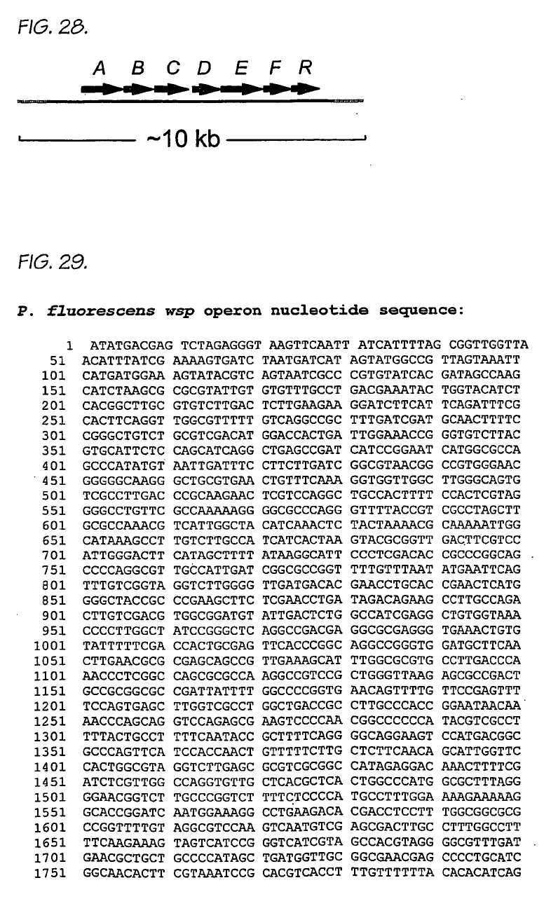

FIG. 28 is a shematic representation of the Pseudomonas fluorescencs wsp operon. The operon consists of seven genes (wspA-F and wspR) located on a ˜10 kb fragment of Pseudomonas fluorescens SBW25 genomic DNA.

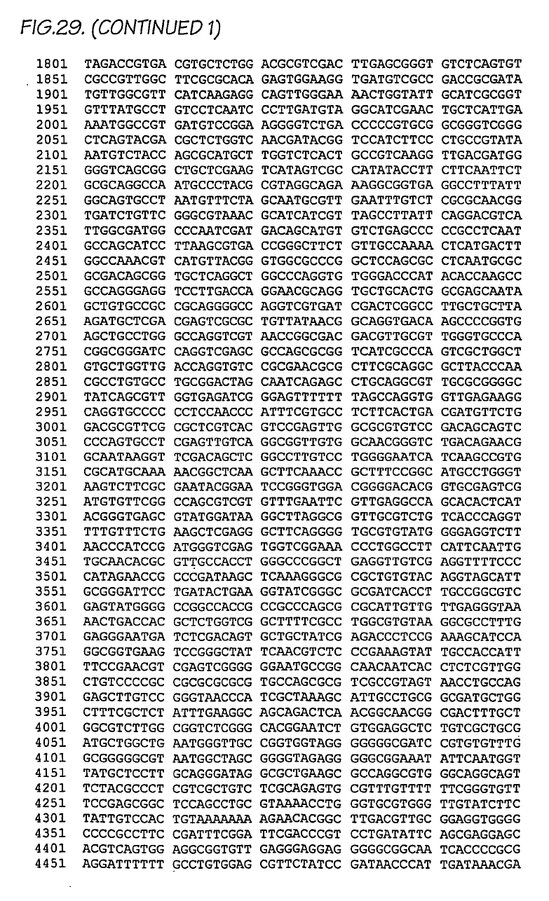

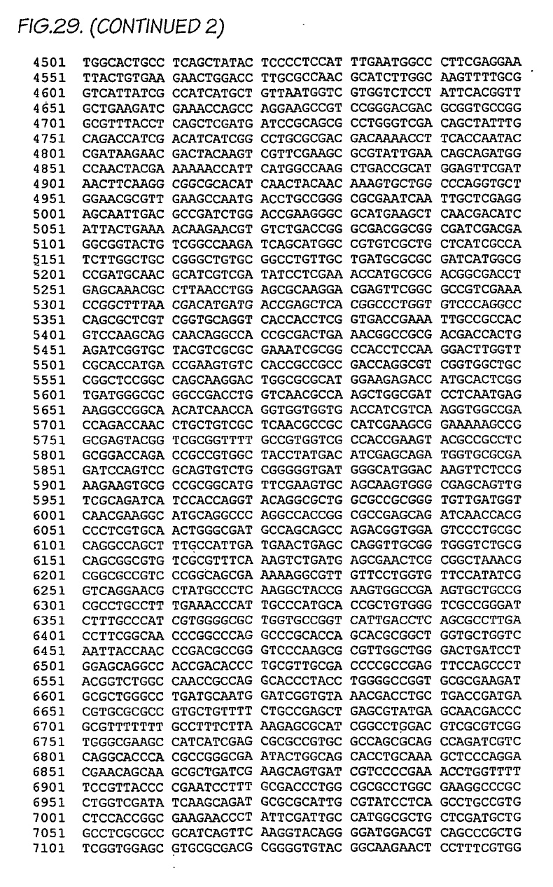

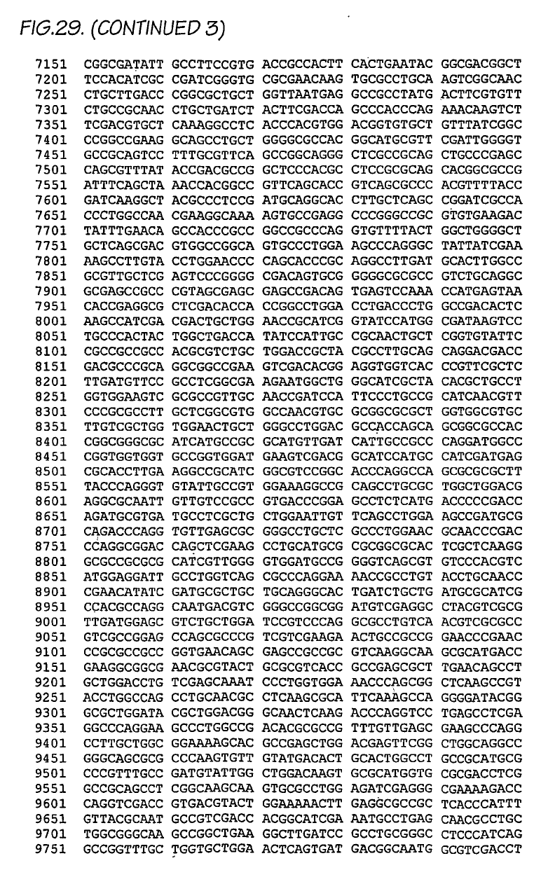

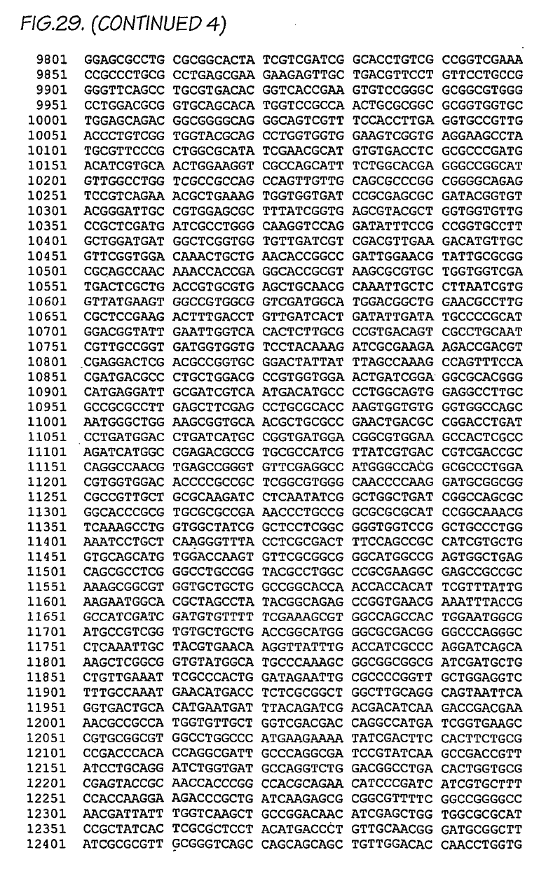

FIG. 29 shows the nucleotide sequence of a fragment of chromosomal DNA from Pseudomonas fluorescens SBW25 including the wsp operon.

A complete annotation for this sequence is given in the accompanying Examples.

FIG. 30 illustrates the predicted translation of the longest open reading frame of the P. fluorescens wspA gene.

FIG. 31 illustrates the predicted translation of the longest open reading frame of the P. fluorescens wspB gene.

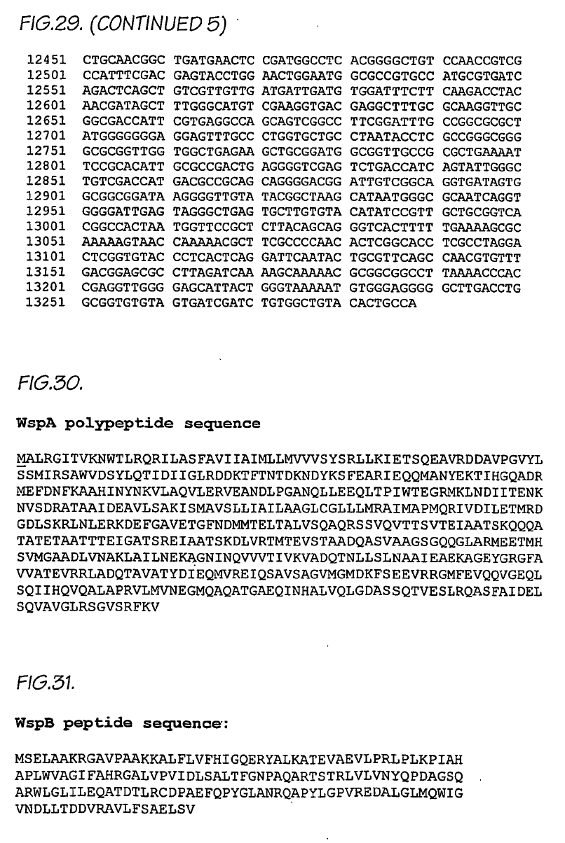

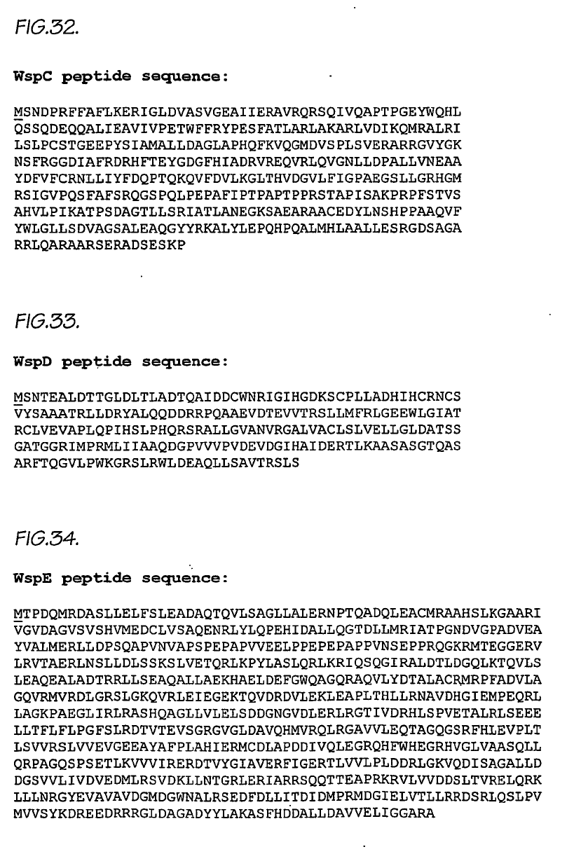

FIG. 32 illustrates the predicted translation of the longest open reading frame of the P. fluorescens wspC gene.

FIG. 33 illustrates the predicted translation of the longest open reading frame of the P. fluorescens wspD gene.

FIG. 34 illustrates the predicted translation of the longest open reading frame of the P. fluorescens wspE gene.

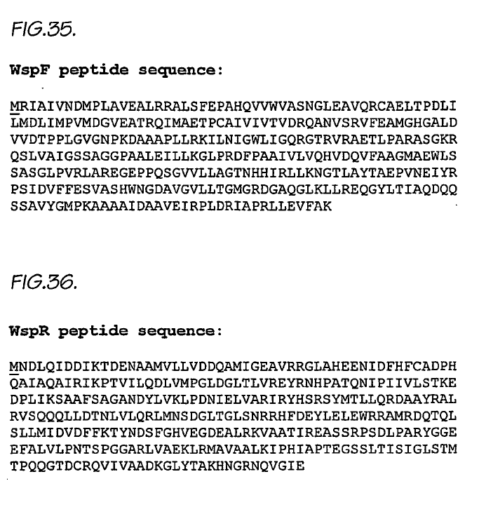

FIG. 35 illustrates the predicted translation of the longest open reading frame of the P. fluorescens wspF gene.

FIG. 36 illustrates the predicted translation of the longest open reading frame of the P. fluorescens wspR gene.

Strain Nomenclature

Throughout the present application various abbreviations are used for P. fluorescens SBW25 strains. The wild-type strain is sometimes referred to as “SM” because of its smooth colony morphology. The Wrinkly Spreader strain expresses glucan-like polysaccharide (GLP) and is often referred to as “WS” because of its wrinkled colony morphology. A variety of WS derivatives are also used and in general they are deficient in GLP production. They are specifically referred to when necessary using numbers, e.g. WS-4, WS-13 etc. In some cases, the same genetic mutation is present in both SM and WS genotypes. In this case, SM genotypes are referred to using the same numbering or naming system as for the original WS mutants (e.g. SM-13), or SM or WS are added to the genotype (e.g. SM wspΔ or WS wspΔ).

### Example 1

Obtaining Glucan-Like Polysaccharide Over-Producing P. fluorescens.

The following example uses the wild type P. fluorescens strain SBW25 which may be isolated from sugar beet leaves, as described by Rainey, P. B. & Bailey, M. J., 1996, Mol. Microbiol., 19: 521-533, and propagated in King's medium B (KB). P. fluorescens SBW25 is also freely available from the culture collection at the Department of Plant Sciences, University of Oxford, Oxford, UK.

In order to obtain polysaccharide producing mutants the ancestral strain SBW25 was allowed to evolve in a static broth environment for 5 days. The static broth environment is typically 6 ml KB medium contained in a 25 ml microcosm at 28° C. Populations are typically founded from single ancestral ‘smooth’ (SM morph) cells (see Rainey & Travisano, 1998, Nature, 394: 69-72 for illustrations of the principal morph classes of P. fluorescens SBW25). Microcosms are incubated without shaking to produce a spatially heterogeneous environment. After 5 days the population shows substantial phenotypic diversity which is easily seen after plating to single colonies on KB agar. During incubation in the static broth culture there is a strong selection for mutants that over-produce glucan-like polysaccharide. These mutants are clearly visible on plating because of their wrinkly spreader colony morphology.

### Example 2

Protocol for Polysaccharide Extraction.

The following protocol may be used for small-scale extraction of the glucan-like polysaccharide of the invention. The protocol as followed up until dehydration will preserve the structure of the polysaccharide network; dehydration or denaturation (by heating or using solvents) may destroy the network. The procedure is designed to first lyse cells, and then to remove proteins through extended proteolysis. Since the procedure involves repeated buffer changes, a lot of the soluble material will be removed from the final sample.

Colonies:

1. Grow up WS colonies on hard LB agar plates (2-3 days incubation): 5 μl o/n culture dotted onto a hard (1.5% agar) LB plate, four dots per plate, incubated at 28° C. for 2-3 days (the colonies should be about 20 mm wide before harvest).

2. Add 2 ml LB broth to plates and leave to stand for 10 min.

3. Roll off colonies and transfer to a small petri dish, remove broth and transfer to a 5 ml Bejou tube.

Mats:

1. Grow standard microcosms (2-3 days incubation): 60 μl o/n culture into 6 ml KB, lid on lightly. Incubate at 28° C. without shaking.

2. Carefully tip the mat onto a petri dish. Drain off excess liquid and transfer mat to a 5 ml Bejou tube.

Preparation:

1. Lyse sample overnight at 37° C. in 5 ml Lysis solution I (with fresh lysozyme)

2. Remove 4 ml of lysozyme buffer.

3. Add DNAase and RNAase. Incubate for 30 min at 37° C.

4. Cool sample on ice for 5 min.

5. Incubate colony for 24 hr in 5 ml Lysis solution II (with fresh Protease K). Samples should become transparent with trapped air bubbles.

6. Transfer the glucan-like polysaccharide (GLP) using a P1000 tip cut off at the end (approx. 500 μl) into 5 ml 0.1% SDS for 30 min. at 37° C. Repeat twice more.

7. Transfer GLP into 5 ml water at RT for five minutes. Repeat four times.

8. Store at 4° C. until needed.

EtOH/dehydration:

1. Wash twice with 50% EtOH for 10 min.

2. Wash twice with 70% EtOH for 10 min.

3. Wash twice with 100% EtOH for 10 min.

4. Transfer GLP into pre-weighed eppendorf tube. Add 1 ml 100% EtOH.

5. Dry at 80° C. o/n.

6. Weigh.

Alternative: Freeze Drying

1. Freeze dry sample directly.

Solutions:

| | |
|---|---|
| Lysis Solution I | 20 mM Tris · HCl pH 8.0 |
| | 5 mM MgC12 |
| | 0.5% Sarkosyl (Sigma) |
| | 1 mg/ml lysozyme (fresh) |
| Lysis Solution II | 500 mM EDTA pH 9.0 |
| | 1% Sarkosyl |
| | 1.5 mg/ml Protease K |

Notes:

Each mat is approximately 0.4 g wet weight, with about 0.02 g or less dry weight. The lysis step should not be omitted as going straight to the protease incubation results in a very messy mat/colony which is not transparent. The DNase/RNase treatment may not be necessary where the polysaccharide preparation is to be used for analytical purposes, since nucleic acids are unlikely to interfere with polysaccharide assays. At each stage so long as there is an obvious mat/colony or gloopy mass, then dialysis against water or a new buffer is a good way of clearing the polysaccharide of digested material. Before the gloopy mass is moved by pipetting, the sample should be left to cool at 4° C. or on ice (especially if incubations have been at 37° C.). Alternatively, polysaccharide might be by isolated by extraction in 1% SDS and centrifugation: after incubation at 37° C. followed by centrifugation at 12K, the polysaccharide will pellet; boil the pellet in more SDS buffer and re-centrifuge; this time the polysaccharide will be in solution. If the polysaccharide concentration is high enough, cooling of the sample on ice should allow it to form transparent ‘clouds’. Alternatively, the sample could be dried and resuspended in a small amount of water where the polysaccharide should form a gel (the sample should be boiled first and then allowed to set). Once in this form, the SDS can be dialysed out. An important feature of the purification step is that it generates a polysaccharide film.

### Example 3

Analysis of a Glucan-Like Polysaccharide

An example of glucan-like polysaccharide produced from a single type of exopolysaccharide producing strain was subjected to composition and linkage analysis using a range of techniques. The strain used was a wrinkly-spreader strain of P. fluorescens isolated using the procedure described in Example 1. It is, however, to be understood that this strain is only one example of the wide range of exopolysaccharide producing strains provided by the invention, each of which may produce polysaccharides which differ slightly in terms of precise structure and composition. The results of analysis of the polysaccharide produced by this strain are therefore not to be construed as limiting to the invention to polysaccharides of this precise structure and composition.

The polysaccharide was purified using the procedure described in Example 2 and subjected to the following analyses:

(1) MALDI-TOF Mass Spectrometry

The polysaccharide material was digested with cellulase and subjected to MALDI-TOF mass spectrometry. A specific peak corresponding to a hexose heptamer was identified by comparison to dextran oligosaccharides.

(2) Acid Hydrolysis

The polysaccharide material was subjected to acid hydrolysis which identified glucose as the major sugar residue. Significant amounts of glucose were only released after pre-treatment in 12M sulphuric acid at 25° C. for 30 minutes. This treatment disrupts cellulose fibres, making the individual cellulose chains more susceptible to subsequent hydrolysis when diluted to 0.5M sulphuric acid.

The results of these analyses strongly suggest that a polymer of glucose linked by β(1-4)-D-glycosidic bonds is the major structural element of the exopolysaccharide produced by a WS strain of P. fluorescens. This may be a cellulose or a cellulose-like polymer which, for example, may contain additional branched or modified sugar residues.

(3) Composition and Linkage Analysis and NMR

Composition and linkage analysis was carried out on freeze-dried samples of polysaccharide from the WS strain. For compositional analysis, the samples were hydrolyzed using freshly prepared 1M methanolic-HCl for 16 hours at 80° C. The released sugars were derivatized with Tr-Sil and the sample was analyzed by GC-MS using a Sp2330 Supelco column. Myo-inositol was also added to the sample as an internal standard. For linkage analysis, the sample was methylated using the NaOH/Mel method (Ciucanu and Kerek, 1984: Ciucanu, I., and F. Kerek, F. (1984). A simple and rapid method for the permethylation of carbohydrates. Carbohydr. Res. 131: 209-217.). The methylated sample was hydrolyzed in 2M TFA at 121° C. for two hours and the hydrolyzed carbohydrate was reduced with sodium borodeuteride at room temperature. The product was acetylated using acetic anhydride at 120° C. for three hours. The derivatized sample was then analyzed by GC-MS. Myo-inositol was added to the sample prior to the reduction step as an internal standard.

NMR analysis used freeze-dried samples. The freeze-dried samples were deuterium-exchanged by repeated evaporation from CD₃OD and dissolved in 0.5 mL of CD₃OD. A 1-D proton spectrum was acquired on a Varian Inova 500 MHz spectrometer at 308° K (35° C.). Proton chemical shifts were measured relative to internal TMS standard (d=0.000 ppm).

Initial chemical analysis of this material showed that the sample contained ˜30% soluble carbohydrates. Of this fraction, composition analysis identified substantial amounts of rhamnose (Rha) and glucose (Glc), as well as trace amounts of fatty acids (Table 1). Linkage data showed two main carbohydrate components, rhamnose and glucose, but N-acetyl glucosamine (GlcNAc), N-acetyl fucosamine (FucNAc) and 3-deoxy-D-amino-2-octulosonic acid (Kdo) were not detected in this experiment. However, these residues are often easily destroyed and are more difficult to observe by linkage analysis than by composition analyses. The sample was treated differently in the composition and linkage analyses (solubilisation and cleavage with HCl/Methanol vs. NaOH/Methanol) and the ratio of rhamnose:glucose identified also varied (0.60 vs. 0.15, respectively). This implies that different fractions of the sample were solubilised by each of these treatments, and that a large fraction of the sample remained insoluble. Finally, the ¹H NMR indicated the presence of alkylated groups in the 1-3 ppm and 4-4.2 ppm regions. The signal from the 1-3 ppm region confirms the fatty acids detected by the composition data. In conclusion, the ¹H NMR data correlated well with the composition data showing the presence of fatty acids and carbohydrates.

TABLE 1

| Residue | % present |
|---|---|
| Compositional Analysis:¹ | |
| Rhamnose (Rha) | 28.7 |
| Xylose (Xyl) | 1.5 |
| 3-deoxy-D-amino-2-octulosonic acid (Kdo) | 6.5 |
| Glucose (Glc) | 48.0 |
| N-acetyl glucosamine (GlcNAc) | 7.9 |
| N-acetyl fucosamine (FucNAc) | 7.4 |
| Linkage Analysis:² | |
| Terminal Rhamnose (t-Rha) | 2.3 |
| 2-Rhamnose (2-Rha) | 6.7 |
| 3-Rhamnose (3-Rha) | 3.9 |
| Terminal Glucose (t-Glc) | 4.9 |
| 4-Glucose (4-Glc) | 82.2 |
| 2,4 Glucose (2,4-Glc) | Trace |
| 4,6 Glucose (4,6-Glc) | Trace |

1. The sample also showed some β-OH C10:0, β-OH C12:0, C16:0 and C16:1 fatty acids.

2. The linkage data only showed two main carbohydrate components, Rha and Glc; GlcNAc, FucNAc and Kdo were not detected in this experiment. These residues are often easily destroyed and are more difficult to observe by linkage analysis.

Total Carbohydrate Analysis

The following analysis was carried out in order to look at the variation of polysaccharide material from a number of different variants of P. fluorescens (SM, WS, WS-6, WS-18 and JB01).

Biofilm material from 48 SM, WS, WS-6, WS-18 and JB01 microcosms were extracted to isolate total carbohydrate for analysis. WS-6 and WS-18 are strains which express unmodified glucan-like polysaccharide, SM does not express the glucan-like polysaccharide, JB01 expresses glucan-like polysaccharide. In general, the composition of all five samples looked very similar, and in each, Rha, Kdo, Glc, GalNAc (N-acetyl galactosamine), GlcNAc and FucNAc were identified (Table 2).

TABLE 2
Mole (%) and Ratio (to Kdo)

| Residues | SM | | WS | | WS-6 | | WS-18 | | JB01 | |
|---|---|---|---|---|---|---|---|---|---|---|
| Rha | 11.5 | 1.69 | 14.6 | 4.56 | 12.2 | 3.13 | 17.2 | 5.06 | 23.9 | 5.43 |
| Kdo | 6.8 | 1.00 | 3.2 | 1.00 | 3.9 | 1.00 | 3.4 | 2.00 | 4.4 | 1.00 |
| Glc | 40.3 | 5.93 | 58.4 | 18.25 | 51.8 | 13.28 | 50.1 | 14.74 | 35.1 | 7.98 |
| GalNAc | 2.2 | 0.32 | 0.8 | 0.25 | 3.0 | 0.77 | 1.9 | 0.56 | 3.2 | 0.73 |
| GlcNAc | 24.8 | 3.65 | 13.3 | 4.16 | 18.0 | 4.62 | 18.0 | 5.29 | 18.1 | 4.11 |
| FucNAc | 14.4 | 2.12 | 9.7 | 3.03 | 11.1 | 2.85 | 9.2 | 2.71 | 15.3 | 3.48 |

The overall conclusion from the structural and compositional analysis was that the glucan-like polysaccharide was predominantly a substituted β (1-4) glucan. Furthermore, it appears that the degree and type of substitution can vary as the different bacterial variants gave rise to polysaccharide analyses that, while consisting of predominantly the same residues, gave different ratios of the sugar monomeric constituents.

### Example 4

WspR Overexpression

(1) Over-Expression of wspR-12 Causes GLP Production to be Switched on, but Requires NaCl:

To over-express the gene wspR-12 was amplified by PCR from the SBW25 genome and a ribosome binding site (GAGGA) added 9 nucleotides from the ATG start of the open reading frame. This was then cloned into plasmid pVSP61 (gift from Steve Lindow, Berkeley) where the wspR gene was expressed from a constitutive Plac promoter. When wspR-12 was over-expressed in ancestral SBW25 no effects on GLP production were noted, however, it was observed that GLP production can be triggered by plating SBW25 containing over-expressed wspR-12 onto LB (No GLP production on KB). The critical ingredient addition was found to be NaCl. The NaCl-dependent effect is only observed in SBW25 containing over-expressed wspR-12.

(2) Variant Alleles of wspR have Radical Effects on GLP Production:

Several alleles of wspR which have radically different effects on GLP production were produced by PCR-mutagenesis. These radical effects were seen following over-expression of the variant allele in either the ancestral SM genotype or the evolved WS genotype (see above for details of over-expression). WspR-19, when over-expressed in the ancestral (non-GLP producing) genotype, results in constitutive, signal-independent, production of GLP. This allele is highly significant in terms of GLP production. The ancestral genotype produces little or no GLP during ordinary growth, but by introducing wspR-19 in the cell a GLP-over-producing strain is generated. There is the potential to use this a gene to activate GLP production in other Pseudomonas strains that are capable of producing GLP, but fail to do so in the laboratory. The mutation in wspR-19 is at the very end of the N-terminal domain. WspR-14 has a similar effect, but the effect is not as strong as wspR-19. The mutation in wspR-14 is in the linker region. The remaining wspR alleles all have dominant-negative effects on GLP production, i.e., in a WS genotype they switch OFF GLP production. All these mutations are in the C-terminal domain.

Summary of wspR alleles:—

wspR-5: Switches GLP production OFF when over-expressed in a GLP-producing WS genotype.

wspR-9: Switches GLP production OFF when over-expressed in a GLP-producing WS genotype.

wspR-12: (wild-type) Over-expression causes GLP production to be switched on, but requires NaCl.

wspR-13: Switches GLP production OFF when over-expressed in a GLP-producing WS genotype.

wspR-14: Switches GLP production ON when over-expressed.

wspR-19: Switches GLP production ON when over-expressed.

### Example 5

Assays for Inhibitors of Glucan-Like Exopolysaccharide Production, Bacterial Attachment and Biofilm Development

There are a number of variations possible on assays that can be used to screen for chemicals which will inhibit bacterial attachment, biofilm development and polysaccharide production. In essence, they all rely on the production of glucan-like polysaccharide by WS or a modified SM strain (glucan-like polysaccharide production is regulated by the same factor which regulates attachment; attachment and biofilm development in SBW25 requires glucan-like polysaccharide production).

Below are the descriptions of the two basic assays, one for agar plates and the other for liquid cultures:

Assay 1: Agar Plate Assay for Polysaccharide Production.

1. Prepare overnight cultures of WS and SM.

2. Dot 5 pl aliquots of the overnight WS and SM cultures onto agar plates containing 0.001% Congo Red plus the test chemical at various concentrations (including one plate with no test chemical as a control). Incubate overnight at 28° C.

3. Score glucan-like polysaccharide production by uptake of Congo Red stain by the WS colony. If it is red, glucan-like polysaccharide production has not been affected. If it is white, glucan-like polysaccharide production has been preveneted. The SM colony should be white; if the colony has not developed, then the test chemical at the concentration used is toxic to the growth of the bacteria.

Assay 2: Liquid Broth Assay for Attachment and Biofilm Production

1. Prepare overnight cultures of WS and SM.

2. Add 100 pl of overnight WS and SM cultures to 30 ml glass vials containing 6 ml KB broth (standard microcosms described by Rainey and Travisano, 1998 [Rainey, P. B. and Travisano, M. (1998). Adaptive radiation in a heterogeneous environment. Nature 394: 69-72.] plus the test chemical at various concentrations (including one set with no test chemical as a control). Incubate at 28° C. without shaking for 24-48 hours.

3. Score attachment and biofilm development:

a. Visually inspect the SM vials. If the test chemical is not toxic, the SM cultures should be cloudy and the meniscus region should be marked with a faint grime or ring (the control vial with SM should be cloudy). If the broth is clear, then there has been no growth of the culture and the test chemical at the concentration used is toxic to the growth of the bacteria.

b. Visually inspect the WS vials. The control vial with WS should contain an obvious biofilm (mat) floating at the top of the vial connected to the glass walls. If the test chemical affects cellulose production, then the mat should be reduced in volume and thickness compared to the control WS vial. If the test chemical prevents cellulose production, then there will be no biofilm present at all.

c. In order to test for attachment, follow this procedure:

i. Swirl the vial and quickly tip all material out of the vial.

ii. Add 6 ml KB both back into the vial, replace lid and shake vigourously for 30 sec. before tipping out liquid.

iii. Repeat (ii) twice more. Allow vials to train upside down.

iv. Visually inspect the vials in the region of the original meniscus. A faint grime mark should be visible for the SM control vial, and a more obvious ring for the WS control (the meniscus is the site of bacterial attachment to the glass vial). Compare these rings with the test chemical vials to determine the effect of the chemicals on attachment.

v. The visual inspection can be quantified using Crystal Violet staining:

a. Add 1 ml 0.1% Crystal Violet solution to each vial and roll so that the dye covers the meniscus region for two minutes. Allow the vials to sit for a few minutes then remove all liquid from the bottom using a pipette.

b. Add 2 ml water to each vial and roll the vial to was the stained region. Allow the water to drain to the bottom and remove with a pippette.

c. Repeat (b) twice. Allow the vials to drain upside down.

d. Add 2 ml 90% EtOH to each vial, replace the lid and vortex or shake vigourously untill all stain is washed from the glass.

e. Remove EtOH/stain and spin at 13K to pellet any debris. Determine the optical density at 570 nm of, the liquid (OD570), and compare against appropriate standards (Crystal violet in 90% EtOH).

f. Samples from vials with high OD570 readings indicate that significat bacterial attachment has taken place. Low values indicate poor or no attachment.

Construction/Isolation of Bacterial Strains: P. fluorescens wspA-FΔ:

This strain was constructed using a standard allelic-replacement technique. The polymerase chain reaction (PCR) was used to amplify two pieces of DNA from the chromosome of P. fluorescens. The upstream fragment ‘A’ included sequences upstream of the wsp operon, and extended to the beginning of the operon, so that it included the promoter and start codon of wspA. The downstream fragment ‘B’ began with the start codon of wspR and finished downstream of the end of wspR. The two PCR fragments were annealed using strand-overlap extension PCR(SOE-PCR) in such a manner that the start codon of wspA in fragment ‘A’ was joined to the start codon of wspR in fragment ‘B’ (so that the wsp promoter and wspA start codon were now in frame with the wspR coding sequence). The new ‘A-B’ fragment was cloned into a suitable suicide vector, and then transferred into P. fluorescens SBW25 strains SM (the wild type strain) and WS (the wrinkly spreader strain). Appropriate co-integrant strains were isolated using the antibiotic resistance of the suicide plasmid. These strains were allowed to grow in the absence of selection to allow a second recombination event to occur and the resultant loss of the suicide vector and original wsp sequences. The presence of the new wspA-FΔ sequence was determined by PCR. These strains have lost wspA-F, but still retain and express wspR. Strains: P. fluorescens SM wspA-FΔ P. fluorescens WS wspA-FΔ.

P. fluorescens wspΔ:

This strain was constructed using the SOE-PCR method described above for P. fluorescens wspA-FΔ, except in this case, all of the wsp sequences were deleted. Fragment ‘B’ included only sequences after the end of the wspR coding sequence. Strains: P. fluorescens SM wspΔ P. fluorescens WS wspΔ.

P. aeruginosa PA01 wspD:

This strain was constructed as for P. fluorescens wspΔ except that the wsp sequences used to design the PCR primers were all derived from the P. aeruginosa PA01 genome (using the wsp-like operon sequence; available from public databases), not the sequences from P. fluorescens.

Complete Sequence Annotation for the P. fluorescens wss Operon.

Set out below is a general description of the genes identified in the wss operon sequence illustrated in FIG. 2 (SEQ ID NO:1). The wss operon encoding the cellulose biosynthetic genes and associated genes, is located approximately between 2,200-18,000 bp of the sequence shown in FIG. 2 (SEQ ID NO:1). The gene co-ordinates for the nine genes (wssA-J) of the operon are listed below. For each gene the positions of the first potential start codon (ATG or GTG [M, methionine or V, valine. N.B. Translation might begin from a GTG codon. However, the first residue would still be a methionine. In order to easily place the peptide sequences with the DNA sequences, GTG-encoded start codons have been left as ‘V’ in the peptide sequences] and the first in-frame stop codon are given. The first potential ATG start codon is also marked (M). A comment about the closest known homologue to each gene is given, along with the probable role of each protein.

2251-3478 wssA Coding Region (Including Upstream Elements).

2251-2358 Upstream region; rich in runs of A.

2309-2358 Predicted promoter region for wssA (5′-CATCAAAATA CTGACACCAT CATTGTGATA TCACAGAATG AGCCCGACAC-3′; A is the predicted transcription start site).

2425-2430 Possible ribosome binding site (RBS) (5′-AGGTGG-3′).

2444-2446 GTG start codon: this is the first possible start codon for WssA. This would produce a protein of 344 amino acids. This start codon is preferred to the first in-frame ATG codon on the basis of E. coli YhjQ homology. WssA is a MinD homologue and contains an ATP-binding motif.

2876-2878 ATG start codon: this is the first in-frame ATG start codon for WssA. This would produce a protein of 200 amino acids.

3476-3478 TGA stop codon: end of WssA.

3475-5694 wssB Coding Region.

3475-3477 ATG start codon: this is the first possible start site for WssB. This would produce a protein of 739 amino acids. WssB is a A. xylinus BcsA (cellulose synthase A subunit) and E. coli YhjO homologue.

5692-5694 TGA stop codon: end of WssB.

5884-7953 wssC coding region.

5884-5886 GTG start codon: this is the first possible start codon for WssC. This would produce a protein of 689 amino acids. WssC is a A. xylinus BcsB (cellulose synthase B subunit) and E. coli YhjN homologue.

6148-6150 ATG start codon: this is the first in-frame ATG start codon for WssC. This would produce a protein of 601 amino acids.

7951-7953 TGA stop codon: end of WssC.

7884-9146 wssD Coding Region.

7884-7886 GTG start codon: this is the first possible start codon for WssD. This would produce a protein of 436 amino acids. WssD shares homology with D-family cellulases often found associated with cellulose synthases. WssD is a A. xylinus CMCase and E. coli YhjM homologue.

7950-7952 ATG start codon: this is the first in-frame ATG start codon for WssD. This would produce a protein of 398 amino acids.

9144-9146 TAG stop codon: end of WssD.

9128-12967 wssE coding region.

9128-9130 ATG start codon: this is the first possible start codon for WssE. This would produce a protein of 1279 amino acids. WssE is a A. xylinus BcsC (cellulose synthase C subunit) and E. coli YhjL homologue.

12965-12967 TGA stop codon: end of WssE.

12984-13649 wssF Coding Region.

12984-12986 ATG start codon: this is the first possible start codon for WssF. This would produce a protein of 221 amino acids. WssF is a A. xylinus BcsX homologue, required for cellulase expression.

13647-13649 TGA stop codon: end of WssF.

13649-14314 wssG coding region.

13649-13651 ATG start codon: this is the first possible start codon for WssG. This would produce a protein of 221 amino acids. WssG is a P. aeruginosa AlgF homologue, required for the acetylation of alginate.

14312-14314 TGA stop codon: end of WssG.

14332-15738 wssH Coding Region.

14332-14334 ATG start codon: this is the first possible start codon for WssH. This would produce a protein of 468 amino acids. WssH is an A. vineladii AlgI and P. aeruginosa AlgI homologue, required for the acetylation of alginate.

15736-15738 TAA stop codon: end of WssH.

15751-16875 wssI coding region.

15751-15753 ATG start codon: this is the first possible start codon for WssI. This would produce a protein of 374 amino acids. WssI is an A. vineladii AlgV/X and P. aeruginosa AlgJ/X homologue, required for the acetylation of alginate.

16873-16875 TAA stop codon: end of WssI.

16938-17912 wssJ Coding Region.

16938-16940 GTG start codon: this is the first possible start codon for WssJ. This would produce a protein of 324 amino acids.

17052-17054 ATG start codon: this is the first in-frame ATG start codon for WssJ. This would produce a protein of 286 amino acids. WssJ is a WssA homologue, and contains an ATP-binding motif.

17910-17912 TAG stop codon: end of WssJ.

17913-20306 Downstream region of the wss operon (end of operon, no further significant coding regions).

Complete Sequence Annotation for the Pseudomonas fluorescens wsp Operon

Set out below is a general description of the genes identified in the wsp operon sequence illustrated in SEQ ID NO: 27. The sequence shown as SEQ ID NO: 27 is contiguous piece of DNA of 13,288 bp from Pseudomonas fluorescens SBW25. The wsp operon encodes a chemotaxis-like operon of seven genes, wspA-F and wspr. A schematic arrangement of the operon is shown as FIG. 28.

The gene co-ordinates for the seven genes of the operon are listed below. For each gene the predicted polypeptide sequence is given, from the first potential start codon [ATG encoding M, methionine] to the first in-frame stop codon. A comment about the closest homologue to each gene is given, along with the probable role of each protein.

4535-6178 wspA Coding Region.

4535-4537 ATG start codon: this is the first possible start codon for WspA. This would produce a protein of 547 amino acids. WspA is a MCP homologue. The deduced amino acid sequence of WspA exhibited significant similarity to methyl-accepting chemotaxis proteins (MCPs), sensory transducers involved in bacterial chemotaxis and motility. The highest similarity was shared with the probable chemotaxis transducer PA3708 from Pseudomonas aeruginosa PA01 (accession C83184) (E-value=1e-167; Identities 60%; Positives 70%) and with a chemotaxis transducer from the plasmid pMLb of Mesorhizobium loti (accession NP_{—}109390) (E-value=1e-120; Identities 45%; Positives 59%).

6176-6178 TGA stop codon: end of WspA.

6178-6690 wspB Coding Region.

6178-6180 ATG start codon: this is the first possible start site for WspB. This would produce a protein of 170 amino acids. WspB is a CheWI homologue. The deduced amino acid sequences of WspB is similar to the chemotactic protein CheW, involved in the transmission of sensory signals from bacterial chemoreceptors (MCPs) to the regulatory components that control chemotaxis and motility. WspB shared highest similarity with the hypothetical protein PA3707 from Pseudomonas aeruginosa PA01 (accession B83184) (E-value=1e-43; Identities 60%; Positives 71%). Similarity was also high with a hypothetical protein from the plasmid pMLb of Mesorhizobium loti (accession NP_{—}109389) (E-value=6e-28; Identities 42%; Positives 61%), with the chemotaxis protein CheW from Rhizobium meliloti (accession Q52881) (E-value=5e-07; Identities 28%; Positives 50%) and other CheW-like bacterial proteins.

6688-6690 TGA stop codon: end of WspB

6687-7946 wspC Coding Region.

6687-6689 ATG start codon: this is the first possible start codon for WspC. This would produce a protein of 419 amino acids. WspC is a CheR homologue. The deduced amino acid sequence of WspC exhibited significant similarity to the chemotaxis protein CheR, belonging to the superfamily of protein-glutamate O-methyltransferases involved in the methylation of MCPs; the methylation state of the MCPs in the cell is crucial for sensory responses and adaptations. The highest similarity was shared with the probable protein methyltransferase PA3706 from Pseudomonas aeruginosa PA01 (accession A83184) (E-value=1e-116; Identities 57%; Positives 66%) and with a methyltransferase from the plasmid pMLb of Mesorhizobium loti (accession NP_{—}109388) (E-value=1e-71; Identities 38%; Positives 52%).

7944-7946 TGA stop codon: end of WspC.

7943-8641 wspD Coding Region.

7943-7945 ATG start codon: this is the first possible start codon for WssD. This would produce a protein of 232 amino acids. WspD is a ChewII homologue. The deduced amino acid sequences of WspD is similar to the chemotactic protein CheW, involved in the transmission of sensory signals from bacterial chemoreceptors (MCPs) to the regulatory components that control chemotaxis and motility. WspD only shared high similarity with the hypothetical protein PA3705 from Pseudomonas aeruginosa PA01 (accession H83183) (E-value=7e-58; Identities 55%; Positives 67%), with a hypothetical protein from the plasmid pMLb of Mesorhizobium loti (accession NP_{—}109387) (E-value=1e-41; Identities 42%; Positives 56%) and with the chemotaxis protein CheW from Rhodobacter sphaeroides (accession Q60251) (E-value=2e-04; Identities 24%; Positives 42%).

8639-8641 TAG stop codon: end of WspD.

8638-10905 wspE Coding Region.

8638-8640 ATG start codon: this is the first possible start codon for WspE. This would produce a protein of 750 amino acids. WspE is a CheA homologue. The deduced amino acid sequence of WspE exhibited similarity to hybrid proteins consisting of the chemotactic histidine kinase CheA and the chemotactic response regulator CheY, both involved in the transmission of sensory signals from the chemoreceptors (MCPs) to the flagellar motors. Following autophosphorylation, the histidine kinase CheA transfers its phosphate group to CheY (or the methylesterase CheB), which in turn interacts directly with the flagellar motor controlling chemotactic behaviour and motility. The highest similarity was shared with the probable chemotaxis sensor/effector fusion protein PA3704 from Pseudomonas aeruginosa PA01 (accession G83183) (E-value=0.0; Identities 66%; Positives 74%) and with a chemotaxis histidine kinase from the plasmid pMLb of Mesorhizobium loti (accession NP 109386) (E-value=0.0; Identities 49%; Positives 62%).

10903-10905 TGA stop codon: end of WspE.

10902-11912 wspF Coding Region.

10902-10904 ATG start codon: this is the first possible start codon for WspF. This would produce a protein of 336 amino acids. WspF is a CheB homologue. The deduced amino acid sequence of WspF exhibited similarity to the chemotaxis protein CheB, belonging to the family of protein-glutamate methylesterases, involved in the demethylation of MCPs following phosphorylation of their response regulator domain by the histidine kinase CheA. Highest similarity was shared with the probable methylesterase PA3703 from Pseudomonas aeruginosa PA01 (accession F83183) (E-value=1e-138; Identities 73%; Positives 84%) and with a methylesterase from the plasmid pMLb of Mesorhizobium loti (accession NP_{—}109385) (E-value=8e-93; Identities 53%; Positives 67%).

11910-11912 TGA stop codon: end of WspF.

11962-12963 wspR Coding Region.

11962-11964 ATG start codon: this is the first possible start codon for WspR. This would produce a protein of 333 amino acids. WspR is a response regulator and exhibited highest similarity with the probable two-component response regulator PA3702 from Pseudomonas aeruginosa PA01 (accession E83183) (E-value=1e-131; Identities 74%;

Positives 85%) and with a regulatory component from the plasmid pMLb of Mesorhizobium loti (accession NP_{—}109384) (E-value=2e-98; Identities 57%; Positives 71%). 12961-12963 TAG stop codon: end of WspR.

### Sequence Listing

SEQ ID NO: 1 Pseudomonas fluorescens wss operon, complete nucleotide sequence

SEQ ID NO: 2 WssA polypeptide sequence

SEQ ID NO: 3 WssB polypeptide sequence

SEQ ID NO: 4 WssC polypeptide sequence

SEQ ID NO: 5 WssD polypeptide sequence

SEQ ID NO: 6 WssE polypeptide sequence

SEQ ID NO: 7 WssF polypeptide sequence

SEQ ID NO: 8 WssG polypeptide sequence

SEQ ID NO: 9 WssH polypeptide sequence

SEQ ID NO: 10 WssI polypeptide sequence

SEQ ID NO: 11 WssJ polypeptide sequence

SEQ ID NO: 12 WspR-12 polypeptide sequence

SEQ ID NO: 13 WspR-12 nucleotide sequence

SEQ, ID NO: 14 WspR-5 polypeptide sequence

SEQ ID NO: 15 WspR-5 nucleotide sequence

SEQ ID NO: 16 WspR-9 polypeptide sequence

SEQ ID NO: 17 WspR-9 nucleotide sequence

SEQ ID NO: 18 WspR-13 polypeptide sequence

SEQ ID NO: 19 WspR-13 nucleotide sequence

SEQ ID NO: 20 WspR-14 polypeptide sequence

SEQ ID NO: 21 WspR-14 nucleotide sequence

SEQ ID NO: 22 WspR-19 polypeptide sequence

SEQ ID NO: 23 WspR-19 nucleotide sequence

SEQ ID NO: 24 pgi nucleotide sequence

SEQ ID NO: 25 mreB nucleotide sequence

SEQ ID NO: 26 E. coli chromosomal sequence, including the yhj operon

SEQ ID NO: 27 Pseudomonas fluorescens Wsp operon, complete nucleotide sequence

SEQ ID NO: 28 WspA polypeptide sequence

SEQ ID NO: 29 WspB polypeptide sequence

SEQ ID NO: 30 WspC polypeptide sequence

SEQ ID NO: 31 WspD polypeptide sequence

SEQ ID NO: 32 WspE polypeptide sequence

SEQ ID NO: 33 WspF polypeptide sequence

SEQ ID NO: 34 WspR polypeptide sequence (wild-type)

1. A glucan-like polysaccharide produced by an exopolysaccharide-producing bacterial strain, said bacterial strain being characterised in that it expresses one or more enzyme-encoding genes of a wss-like operon.

2. A polysaccharide as claimed in claim 1 which is an exopolysaccharide which stains positively with the reagent calcofluor.

3. A polysaccharide as claimed in claim 1 or claim 2 which is a polymer having a polymer backbone comprised mainly of glucose residues linked by glycosidic bonds.

4. A polysaccharide as claimed in claim 3 wherein the polymer backbone contains further non-glucose hexose or derivatised hexose residues.

5. A polysaccharide as claimed in claim 3 or claim 4 wherein one or more of the sugar residues forming the backbone of the polymer are substituted at any position by further hexose or pentose residues, derivatised hexose or pentose residues or other functional groups.

6. A method of isolating an exopolysaccharide-producing bacterial strain, which method comprises the steps of:
(a) growing a wild type bacterial strain, the genome of which comprises a wss-like operon, in a static broth culture;
(b) isolating a variant strain having wrinkly spreader morphology; and
(c) optionally, screening colonies of the wrinkly spreader morph obtained in step (b) for the production of complex polysaccharide.

7. A method as claimed in claim 6 which comprises the step of screening colonies of the wrinkly spreader morph obtained in step (b) for the production of complex polysaccharide by staining with a staining reagent specific for β-linked glucan polysaccharides.

8. A method as claimed in claim 7 wherein the staining reagent is calcofluor.

9. A method as claimed in any one of claims 6 to 8 wherein the wild type bacterial strain is a Pseudomonas strain.

10. A method as claimed in claim 9 wherein the Pseudomonas strain is Pseudomonas fluorescens-SBW25.

11. A method as claimed in any one of claims 6 to 8 wherein the wild type bacterial strain is an E. coli strain.

12. A method as claimed in claim 11 wherein the E. coli strain is an E. coli K12 strain.

13. An exopolysaccharide-producing bacterial strain which is obtainable by the method claimed in any one of claims 6 to 12.

14. A glucan-like polysaccharide produced by a bacterial strain according to claim 13.

15. A method of isolating an exopolysaccharide-producing bacterial strain, which method comprises the steps of:
(a) exposing a wild type bacterial strain, the genome of which comprises a wss-like operon, to a chemical mutagen; and
(b) identifying a mutant which produces polysaccharide.

16. A method as claimed in claim 15 where mutants which produce polysaccharide are identified on the basis of staining with a staining reagent specific for β-linked glucan polysaccharides.

17. A method as claimed in claim 16 wherein the staining reagent is calcofluor.

18. A method as claimed in any one of claims 15 to 17 wherein the wild type bacterial strain is a Pseudomonas strain.

19. A method as claimed in claim 18 wherein the Pseudomonas strain is Pseudomonas fluorescens SBW25.

20. A method as claimed in any one of claims 15 to 17 wherein the wild type bacterial strain is an E. coli strain.

21. A method as claimed in claim 21 wherein the E. coli strain is an E. coli K12 strain.

22. An exopolysaccharide-producing bacterial strain which is obtainable by the method claimed in any one of claims 15 to 21.

23. A glucan-like polysaccharide produced by a bacterial strain according to claim 22.

24. An isolated nucleic acid molecule encoding a WspR protein, said protein comprising the sequence of amino acids illustrated in SEQ ID NO: 12, SEQ ID NO: 14, SEQ ID NO: 16, SEQ ID NO: 18, SEQ ID NO: 20 or SEQ ID NO: 22.

25. An isolated nucleic acid molecule according to claim 24 which comprises the sequence of nucleotides illustrated in SEQ ID NO: 13, SEQ ID NO: 15, SEQ ID NO: 17, SEQ ID NO: 19, SEQ ID NO: 21 or SEQ ID NO: 23.

26. An isolated nucleic acid molecule which is capable of hybridising to the nucleic acid molecule of claim 24 or claim 25 under conditions of high stringency.

27. An isolated WspR protein comprising the sequence of amino acids shown in SEQ ID NO: 12, SEQ ID NO: 14, SEQ ID NO: 16, SEQ ID NO: 18, SEQ ID NO: 20 or SEQ ID NO: 22.

28. An isolated WspR protein which is encoded by a nucleic acid molecule as defined in claim 24 or claim 25.

29. An expression vector comprising the nucleic acid of claim 24 or claim 25 operably linked to nucleic acid sequences which control its expression.

30. A prokaryotic host cell comprising an expression vector according to claim 29.

31. A method of constructing an exopolysaccharide-producing bacterial strain, which method comprises introducing into a host bacterial strain, the genome of which contains a wss-like operon, an expression vector suitable for overexpression of a wild-type WspR protein or an allelic variant thereof which is capable of functioning a positive regulator of the said wss operon.

32. A method as claimed in claim 31 wherein the host bacterial strain is a strain of Pseudomonas.

33. A method as claimed in claim 32 wherein the Pseudomonas strain is Pseudomonas fluorescens SBW25.

34. A method as claimed in claim 31 wherein the host bacterial strain is an E. coli strain.

35. A method as claimed in any one of claims 31 to 34 wherein WspR protein comprises the amino acid sequence illustrated in SEQ ID NO: 12, SEQ ID NO: 20 or SEQ ID NO: 22.

36. An exopolysaccharide-producing bacterial strain which is obtainable by the method of any one of claims 31 to 35.

37. An exopolysaccharide-producing bacterial strain as claimed in claim 36 which further has a mutation in the mreB gene and/or a mutation in the pgi gene.

38. A glucan-like polysaccharide produced by a bacterial strain as claimed in claim 36 or claim 37.

39. An isolated nucleic acid molecule comprising the sequence of nucleotides from position 2444 to position 13649 or from position 2444 to position 17912 of the nucleotide sequence illustrated in SEQ ID NO: 1 or the individual coding regions thereof.

40. An isolated nucleic acid molecule comprising the sequence of nucleotides illustrated in SEQ ID NO: 26 or the individual coding regions thereof.

41. An expression vector comprising the nucleic acid of claim 39 or claim 40 operably linked to regulatory sequences which control expression of the said nucleic acid.

42. An expression vector as claimed in claim 41 wherein the said regulatory sequences comprise an inducible promoter.

43. An expression vector comprising the sequence of nucleotides from position 2200 to position 18000 of the nucleotide sequence illustrated in SEQ ID NO: 1.

44. An exopolysaccharide-producing bacterial strain which is a bacterial host strain comprising an expression vector as claimed in claim 42 or claim 43.

45. An exopolysaccharide-producing bacterial strain which is a bacterial host strain comprising an expression vector as claimed in claim 43 and further comprising nucleic acid encoding a WspR protein.

46. An exopolysaccharide-producing bacterial strain as claimed in claim 45 wherein the WspR protein comprises the sequence of amino acids illustrated in SEQ ID NO: 12, SEQ ID NO: 20 or SEQ ID NO: 22.

47. A glucan-like polysaccharide produced by an exopolysaccharide-producing bacterial strain as claimed in any one of claims 44 to 47.

48. An isolated nucleic acid molecule encoding a WssA protein, said protein comprising the sequence of amino acids from position 2 to position 344 of the amino acid sequence illustrated in SEQ ID NO: 2 or from position 145 to position 344 of the amino acid sequence illustrated in SEQ ID NO: 2.

49. An isolated nucleic acid molecule according to claim 48 which comprises the sequence of nucleotides from position 2444 to position 3478 or from position 2444 to position 3478 of the nucleic acid sequence illustrated in SEQ ID NO: 1.

50. An isolated WssA protein comprising the sequence of amino acids from position 2 to position 344 of the amino acid sequence illustrated in SEQ ID NO: 2 or from position 145 to position 344 of the amino acid sequence illustrated in SEQ ID NO: 2.

51. An expression vector comprising the nucleic acid of claim 48 or claim 49.

52. A prokaryotic host cell comprising an expression vector according to claim 51.

53. An isolated nucleic acid molecule encoding a WssB protein, said protein comprising the sequence of amino acids illustrated in SEQ ID NO: 3.

54. An isolated nucleic acid molecule according to claim 53 which comprises the sequence of nucleotides from position 3475 to position 5694 of the nucleic acid sequence illustrated in SEQ ID NO: 1.

55. An isolated WssB protein comprising the sequence of amino acids illustrated in SEQ ID NO: 3.

56. An expression vector comprising the nucleic acid of claim 53 or claim 54.

57. A prokaryotic host cell comprising an expression vector according to claim 56.

58. An isolated nucleic acid molecule encoding a WssC protein, said protein comprising the sequence of amino acids from position 2 to position 689 of the amino acid sequence illustrated in SEQ ID NO: 4 or from position 89 to position 689 of the amino acid sequence illustrated in SEQ ID NO: 4.

59. An isolated nucleic acid molecule according to claim 58 which comprises the sequence of nucleotides from position 5884 to position 7953 or from position 6148 to position 7953 of the nucleic acid sequence illustrated in SEQ ID NO: 1.

60. An isolated WssC protein comprising the sequence of amino acids from position 2 to position 689 of the amino acid sequence illustrated in SEQ ID NO: 4 or from position 89 to position 689 of the amino acid sequence illustrated in SEQ ID NO: 4.

61. An expression vector comprising the nucleic acid of claim 58 or claim 59.

62. A prokaryotic host cell comprising an expression vector according to claim 61.

63. An isolated nucleic acid molecule encoding a WssD protein, said protein comprising the sequence of amino acids from position 2 to position 436 of the amino acid sequence illustrated in SEQ ID NO: 5 or from position 39 to position 436 of the amino acid sequence illustrated in SEQ ID NO: 5.

64. An isolated nucleic acid molecule according to claim 63 which comprises the sequence of nucleotides from position 7884 to position 9146 or from position 7950 to position 9146 of the nucleic acid sequence illustrated in SEQ ID NO: 1.

65. An isolated WssD protein comprising the sequence of amino acids from position 2 to position 436 of the amino acid sequence illustrated in SEQ ID NO: 5 or from position. 39 to position 436 of the amino acid sequence illustrated in SEQ ID NO: 5.

66. An expression vector comprising the nucleic acid of claim 63 or claim 64.

67. A prokaryotic host cell comprising an expression vector according to claim 66.

68. An isolated nucleic acid molecule encoding a WssE protein, said protein comprising the sequence of amino acids illustrated in SEQ ID NO: 6.

69. An isolated nucleic acid molecule according to claim 68 which comprises the sequence of nucleotides from position 9128 to position 12967 of the nucleic acid sequence illustrated in SEQ ID NO: 1.

70. An isolated WssE protein comprising the sequence of amino acids illustrated in SEQ ID NO:6.

71. An isolated WssE protein which is encoded by a nucleic acid molecule as defined in claim 68 or claim 69.

72. An expression vector comprising the nucleic acid of claim 68 or claim 69.

73. A prokaryotic host cell comprising an expression vector according to claim 72.

74. An isolated nucleic acid molecule encoding a WssF protein, said protein comprising the sequence of amino acids illustrated in SEQ ID NO: 7.

75. An isolated nucleic acid molecule according to claim 74 which comprises the sequence of nucleotides from position 12984 to position 13649 of the nucleic acid sequence illustrated in SEQ ID NO: 1.

76. An isolated WssF protein comprising the sequence of amino acids illustrated in SEQ ID NO: 7.

77. An expression vector comprising the nucleic acid of claim 74 or claim 75.

78. A prokaryotic host cell comprising an expression vector according to claim 77.

79. An isolated nucleic acid molecule encoding a WssG protein, said protein comprising the sequence of amino acids illustrated in SEQ ID NO: 8.

80. An isolated nucleic acid molecule according to claim 79 which comprises the sequence of nucleotides from position 13649 to position 14314 of the nucleic acid sequence illustrated in SEQ ID NO: 1.

81. An isolated WssG protein comprising the sequence of amino acids illustrated in SEQ ID NO: 8.

82. An expression vector comprising the nucleic acid of claim 79 or claim 80.

83. A prokaryotic host cell comprising an expression vector according to claim 82.

84. An isolated nucleic acid molecule encoding a WssH protein, said protein comprising the sequence of amino acids illustrated in SEQ ID NO: 9.

85. An isolated nucleic acid molecule according to claim 84 which comprises the sequence of nucleotides from position 14332 to position 15738 of the nucleic acid sequence illustrated in SEQ ID NO: 1.

86. An isolated WssH protein comprising the sequence of amino acids illustrated in SEQ ID NO: 9.

87. An expression vector comprising the nucleic acid of claim 84 or claim 85.

88. A prokaryotic host cell comprising an expression vector according to claim 87.

89. An isolated nucleic acid molecule encoding a WssI protein, said protein comprising the sequence of amino acids illustrated in SEQ ID NO: 10.

90. An isolated nucleic acid molecule according to claim 89 which comprises the sequence of nucleotides from position 15751 to position 16875 of the nucleic acid sequence illustrated in SEQ ID NO: 1.

91. An isolated WssI protein comprising the sequence of amino acids illustrated in SEQ ID NO: 10.

92. An expression vector comprising the nucleic acid of claim 89 or claim 90.

93. A prokaryotic host cell comprising an expression vector according to claim 92.

94. An isolated nucleic acid molecule encoding a WssJ protein, said protein comprising the sequence of amino acids from position 2 to position 324 or from position 39 to position 324 of the amino acid sequence illustrated in SEQ ID NO: 11.

95. An isolated nucleic acid molecule according to claim 94 which comprises the sequence of nucleotides from position 16938 to position 17912 or from position 17052 to position 17912 of the nucleic acid sequence illustrated in SEQ ID NO: 1.

96. An isolated WssJ protein comprising the sequence of amino acids from position 2 to position 324 or from position 39 to position 324 of the amino acid sequence illustrated in SEQ ID NO: 11.

97. An expression vector comprising the nucleic acid of claim 94 or claim 95.

98. A prokaryotic host cell comprising an expression vector according to claim 97.

99. A glucan-like polysaccharide as defined in any one of claims 1 to 5 which is obtainable by, (a) culturing an exopolysaccharide-producing bacterial strain;
(b) lysing the bacteria overnight at 37° C. in a lysis solution of 20 mM Tris.HCl pH 8.0, 5mM MgCl₂, 0.5% Sarkosyl, 1 mg/ml fresh lysozyme;
(c) treating the lysed sample obtained in step (b) with DNase and RNase; and
(d) incubating the sample obtained in step (c) for 24 hr with a second lysis solution of 500 mM EDTA pH9.0, 1% Sarkosyl, 1.5 mg/ml Proteinase K.

100. A glucan-like polysaccharide as claimed in claim 99 which is obtainable from an exopolysaccharide producing strain as defined in any one of claims 13, 22, 36, 37 or 44-46.

101. A process for obtaining a glucan-like polysaccharide from an exopolysaccharide-producing bacterial strain, said bacterial strain being characterised in that it expresses one or more enzyme-encoding genes of a wss-like operon, the process comprising the steps of growing the exopolysaccharide-producing bacterial strain and isolating the glucan-like polysaccharide produced thereby.

102. A process as claimed in claim 101 wherein the step of isolating the polysaccharide comprises lysing the exopolysaccharide-producing bacterial strain with a lysis solution of 20 mM Tris.HCl pH8.0, 5 mM MgCl₂, 0.5% Sarkosyl, 1 mg/ml fresh lysozyme and incubating the thus sample obtained with a second lysis solution of 500 mM EDTA pH 9.0, 1% Sarkosyl, 1.5 mg/ml Proteinase K.

103. A process as claimed in claim 101 or claim 102 wherein the exopolysaccharide producing bacterial strain is a strain according to any one of claims 13, 22, 36, 37 or 44-46.

104. An isolated nucleic acid molecule comprising the nucleotide sequence illustrated in SEQ ID NO: 24.

105. An isolated nucleic acid molecule comprising the nucleotide sequence illustrated in SEQ ID NO: 25.

106. An isolated nucleic acid molecule encoding a WspA protein, said protein comprising the sequence of amino acids illustrated in SEQ ID NO: 28.

107. An isolated nucleic acid molecule according to claim 106 which comprises the sequence of nucleotides from position 4535 to position 6178 of the nucleic acid sequence illustrated in SEQ ID NO: 27.

108. An isolated WspA protein comprising the sequence of amino acids illustrated in SEQ ID NO: 28.

109. An expression vector comprising the nucleic acid of claim 106 or claim 107.

110. A prokaryotic host cell comprising an expression vector according to claim 111.

111. An isolated nucleic acid molecule encoding a WspB protein, said protein comprising the sequence of amino acids illustrated in SEQ ID NO: 29.

112. An isolated nucleic acid molecule according to claim 111 which comprises the sequence of nucleotides from position 6178 to position 6690 of the nucleic acid sequence illustrated in SEQ ID NO: 27.

113. An isolated WspB protein comprising the sequence of amino acids illustrated in SEQ ID NO: 29.

114. An expression vector comprising the nucleic acid of claim 111 or claim 112.

115. A prokaryotic host cell comprising an expression vector according to claim 114.

116. An isolated nucleic acid molecule encoding a WspC protein, said protein comprising the sequence of amino acids illustrated in SEQ ID NO: 30.

117. An isolated nucleic acid molecule according to claim 116 which comprises the sequence of nucleotides from position 6687 to position 7946 of the nucleic acid sequence illustrated in SEQ ID NO: 27.

118. An isolated WspC protein comprising the sequence of amino acids illustrated in SEQ ID NO: 30.

119. An expression vector comprising the nucleic acid of claim 116 or claim 117.

120. A prokaryotic host cell comprising an expression vector according to claim 119.

121. An isolated nucleic acid molecule encoding a WspD protein, said protein comprising the sequence of amino acids illustrated in SEQ ID NO: 31.

122. An isolated nucleic acid molecule according to claim 121 which comprises the sequence of nucleotides from position 7943 to position 8641 of the nucleic acid sequence illustrated in SEQ ID NO: 27.

123. An isolated WspD protein comprising the sequence of amino acids illustrated in SEQ ID NO: 31.

124. An expression vector comprising the nucleic acid of claim 122 or claim 123.

125. A prokaryotic host cell comprising an expression vector according to claim 124.

126. An isolated nucleic acid molecule encoding a WspE protein, said protein comprising the sequence of amino acids illustrated in SEQ ID NO: 32.

127. An isolated nucleic acid molecule according to claim 126 which comprises the sequence of nucleotides from position 8638 to position 10905 of the nucleic acid sequence illustrated in SEQ ID NO: 27.

128. An isolated WspD protein comprising the sequence of amino acids illustrated in SEQ ID NO: 32.

129. An expression vector comprising the nucleic acid of claim 126 or claim 127.

130. A prokaryotic host cell comprising an expression vector according to claim 129.

131. An isolated nucleic acid molecule encoding a WspF protein, said protein comprising the sequence of amino acids illustrated in SEQ ID NO: 33.

132. An isolated nucleic acid molecule according to claim 131 which comprises the sequence of nucleotides from position 10902 to position 11912 of the nucleic acid sequence illustrated in SEQ ID NO: 27.

133. An isolated WspF protein comprising the sequence of amino acids illustrated in SEQ ID NO: 33.

134. An expression vector comprising the nucleic acid of claim 131 or claim 132.

135. A prokaryotic host cell comprising an expression vector according to claim 134.

136. An isolated nucleic acid molecule comprising the sequence of nucleotides illustrated in SEQ ID NO: 27 or the individual coding regions thereof.

137. A method for identifying inhibitors of bacterial exopolysaccharide production, which method comprises culturing exopolysaccharide producing bacteria in the presence of a test chemical and a dye which specifically stains exopolysaccharide, scoring production of exopolysaccharide by observing uptake of the dye and thereby identifying inhibitors of exopolysaccharide production.

138. A method for identifying inhibitors of bacterial attachment and/or biofilm formation, which method comprises culturing exopolysaccharide producing bacteria in the presence of a test chemical, observing bacterial attachment and/or biofilm formation by visual inspection and thereby identifying inhibitors of bacterial attachment and/or biofilm formation.

139. A method for identifying inhibitors of bacterial attachment and/or biofilm formation, which method comprises culturing a bacterial strain which expresses Wsp homologues required for bacterial attachment and a control strain which is substantially identical except that it does not express Wsp homologues required for bacterial attachment in the presence of a test chemical, observing bacterial attachment and/or biofilm formation by visual inspection and thereby identifying inhibitors of bacterial attachment and/or biofilm formation.

140. A method according to claim 139 wherein the bacterial strain which expresses Wsp homologues required for bacterial attachment is wild type P. aeruginosa PA01 and the control strain which is substantially identical except that it does not express Wsp homologues required for bacterial attachment is P. aeruginosa PA01 wspΔ.

141. A method according to claim 139 wherein the bacterial strain which expresses Wsp homologues required for bacterial attachment is wild-type P. fluorescens SBW25 and the control strain which is substantially identical except that it does not express Wsp homologues required for bacterial attachment is P. fluorescens SBW25 wspΔ.
